(19) United States
(12) Patent Application Publication
(10) Pub. No.: US 2019/0308933 A1
(43) Pub. Date: Oct. 10, 2019

(54) PHENYLAMIDINES AND THE USE THEREOF AS FUNGICIDES

(71) Applicants: Bayer CropScience Aktiengesellschaft, Monheim Am Rhein (DE); Bayer Aktiengesellschaft, Leverkusen (DE)

(72) Inventors: Cyril MONTAGNE, Lyon (FR); Mazen ES-SAYED, Langenfeld (DE); Andreas GÖRTZ, Dormagen (DE); Ulrike WACHENDORFF-NEUMANN, Neuwied (DE)

(73) Assignees: Bayer CropScience Aktiengesellschaft, Monheim Am Rhein (DE); Bayer Aktiengesellschaft, Leverkusen (DE)

(21) Appl. No.: 16/469,586

(22) PCT Filed: Dec. 13, 2017
(86) PCT No.: PCT/EP2017/082602

§ 371 Date: Jun. 13, 2019

(30) Foreign Application Priority Data
Dec. 14, 2016 (EP) 16204169.3

(51) Int. Cl. C07C 257/18; A01N 37/52

(52) U.S. Cl. CPC C07C 257/18; A01N 37/52

(57) ABSTRACT

The present invention relates to compounds of the formula (I), in particular to phenylamidines of the formula (I), to a process for their preparation, to the use of phenylamidines of the formula (I) according to the invention for controlling unwanted microorganisms, in particular phytopathogenic fungi and also to a composition for this purpose, comprising the phenylamidines of the formula (I) according to the invention. Furthermore, the invention relates to a method for controlling unwanted microorganisms, in particular phytopathogenic fungi, characterized in that the compounds of the formula (I) are applied to the microorganisms, in particular to the phytopathogenic fungi and/or in their habitat.

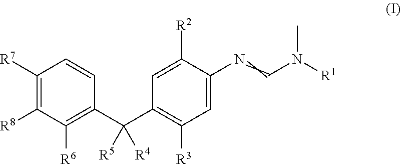

The present invention relates to compounds of the formula (I), in particular to phenylamidines of the formula (I), to a process for their preparation, to the use of phenylamidines of the formula (I) according to the invention for controlling unwanted microorganisms, in particular phytopathogenic fungi and also to a composition for this purpose, comprising the phenylamidines of the formula (I) according to the invention. Furthermore, the invention relates to a method for controlling unwanted microorganisms, in particular phytopathogenic fungi, characterized in that the compounds of the formula (I) are applied to the microorganisms, in particular to the phytopathogenic fungi and/or in their habitat.

WO2000/046184 discloses the use of amidines, including N-methyl-N-methyl-N′-[(4-phenoxy)-2,5-xylyl]-formamidine, as fungicides.

WO2003/093224, WO2007/031512, WO2007/031513, WO2007/031523, WO2007/031524, WO2007/031526, WO2007/031527, WO2007/061966, WO2008/101682, WO2008/110279, WO2008/110280, WO2008/110281, WO2008/110312, WO2008/110313, WO2008/110314, WO2008/110315, WO2008/128639, WO2009/156098, WO2009/156074, WO2010/086118, WO2012/025450, WO2012/090969 and WO2014/157596 disclose the use of arylamidine derivatives as fungicides.

WO2007/031508 and WO2007/093227 disclose the use of arylamidine derivatives as fungicides and insecticides.

WO2003/024219 discloses fungicide compositions comprising at least one N2-phenylamidine derivative in combination with a further selected known active compound.

WO2004/037239 discloses antifungicidal medicaments based on N2-phenylamidine derivatives.

WO2005/089547, WO2005/120234, WO2012/146125, WO2013/136275, and WO2014/037314 disclose fungicide mixtures comprising at least one arylamidine derivative and a further selected known fungicide.

WO2007/031507 discloses fungicide mixtures comprising at least one arylamidine derivative and two other selected known fungicides.

The effectiveness of the phenylamidines described in the prior art as fungicides is good but in many cases the spectrum of action for example in view of the fungicidal efficacy and/or the used application rate needs to be improved. In particular the fungicidal efficacy needs to be improved.

Accordingly, it is an object of the present invention to provide phenylamidines having an improved fungicidal efficacy and to improve the compatibility with plants. In particular, it is an object of the present invention to provide phenylamidines having an improved plant compatibility.

It has now been found that the inventive compounds of formula (I) achieve a higher fungicidal efficacy compared to known phenylamidines. In addition, a broad spectrum of action with respect to the phytopathogenic fungi to be controlled was observed for the inventive compounds of formula (I), i.e. inventive compounds of formula (I) act as fungicides with improved fungicidal efficacy.

Thus, the use of the inventive compounds according to formula (I) contributes considerably to achieving the maximum productivity of crops and therefore finally also safeguards quality and yield within agriculture.

Accordingly, the present invention provides phenylamidines of the formula (I)

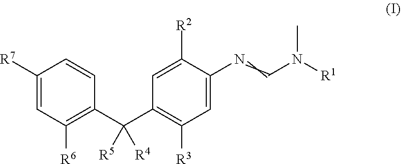

in which
R¹ is selected from the group consisting of C₁-C₈-alkyl, C₃-C₇-cycloalkyl which may be independently non-substituted or substituted by one or more group(s) selected from halogen or C₁-C₈-alkoxy;
R² and R³ are each independently selected from the group consisting of halogen, cyano, C₁-C₈-alkyl, C₃-C₇-cycloalkyl, —O—C₁-C₈-alkyl, C₂-C₈-alkenyl, C₂-C₈-alkynyl, —Si(R^{3a})(R^{3b})(R^{3c}), —C(O)—C₁-C₈-alkyl, —C(O)—C₃-C₇-cycloalkyl, —C(O)NH—C₁-C₈-alkyl, —C(O)N-di-C₁-C₈-alkyl, —C(O)O—C₁-C₈-alkyl, —S(O)ₙ—C₁-C₈-alkyl, —NH—C₁-C₈-alkyl, —N-di-C₁-C₈-alkyl, which may be independently non-substituted or substituted by one or more group(s) selected from halogen or C₁-C₈-alkoxy;
   wherein R^{3a}, R^{3b}, R^{3c} represent independently from each other phenyl or C₁-C₈-alkyl;
   n represents 0, 1 or 2;
R⁴, R⁵, R⁶, R⁷ and R⁸ are each independently selected from the group consisting of halogen, cyano, C₁-C₈-alkyl, C₃-C₇-cycloalkyl, —O—C₁-C₈-alkyl, C₂-C₈-alkenyl, C₂-C₈-alkynyl, —Si(R^{3a})(R^{3b})(R^{3c}), —C(O)—C₁-C₈-alkyl, —C(O)—C₃-C₇-cycloalkyl, —C(O)NH—C₁-C₈-alkyl, —C(O)N-di-C₁-C₈-alkyl, —C(O)O—C₁-C₈-alkyl, —S(O)ₙ—C₁-C₈-alkyl, —NH—C₁-C₈-alkyl, —N-di-C₁-C₈-alkyl, C₆-C₁₄-aryl, which may be independently non-substituted or substituted by one or more group(s) selected from halogen, methyl, halomethyl or C₁-C₈-alkoxy;
   wherein R^{3a}, R^{3b}, R^{3c} represent independently from each other phenyl or C₁-C₈-alkyl;
   n represents 0, 1 or 2;
or in which R⁴ and R⁵ can form, together with the atoms to which they are bonded or with additional atoms chosen from N, O, P and S, a 3- to 7-membered ring selected from the group consisting of cycloalkyl and heterocyclyl, which may optionally be substituted by one or more group(s) selected from halogen, and wherein R⁶, R⁷ and R⁸ are as defined above;
or in which R⁴ and R⁵ together can form a double bonded substituent ═CR⁹R¹⁰, wherein R⁹ and R¹⁰ are each independently selected from the group consisting of H, halogen, Me and Et, and wherein R⁶, R⁷ and R⁸ are as defined above.

The radical definitions specified above can be combined with one another as desired.

The “crossed line” representation of the N—C double bond in formula (I) reflects the possible cis/trans stereochemistry of this bond.

According to the type of substituents defined above, the compounds of the formula (I) have basic properties and can form salts, possibly also internal salts or adducts, with inorganic or organic acids or with metal ions. The compounds of the formula (I) carry amidine groups which induce basic properties. Thus, these compounds can be reacted with acids to give salts, or they are obtained directly as salts by the synthesis.

The salts obtainable in this way likewise have fungicidal properties.

Optionally substituted groups may be mono- or polysubstituted, where the substituents in the case of polysubstitutions may be the same or different.

Furthermore, the present invention provides a process for preparing the phenylamidines according to the invention which comprises at least one of the following steps (a) to (g):
(a) reaction of anilines derivatives of formula (II) to afford derivatives of formula (III) according to the reaction scheme below:

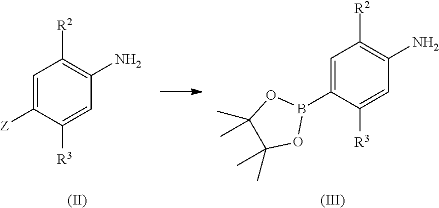

(b) reaction of derivatives of formula (III) with benzyl derivatives of formula (IV) to afford derivatives of formula (V) in accordance with the reaction scheme below:

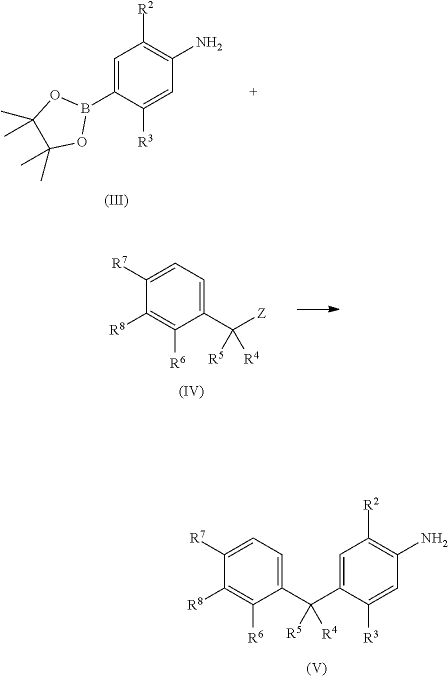

(c) coupling of nitrobenzene derivatives of formula (VI) with boronic acids or esters of formula (VII) to afford alkenyl derivatives of formula (VIII) according to the reaction scheme below:

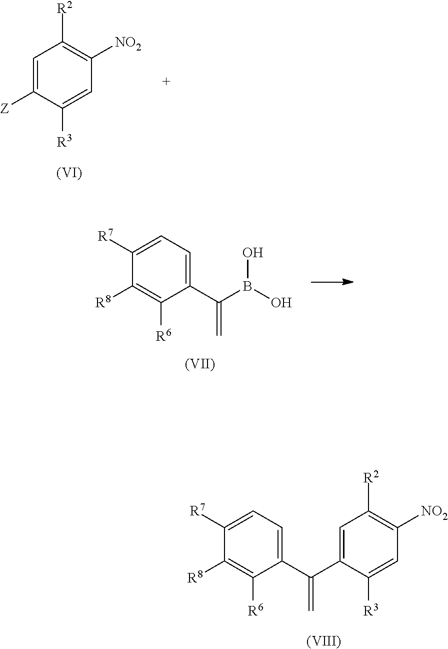

(d) reaction of alkenyl derivatives of formula (VIII) to afford cyclopropyl derivatives of formula (IX) according to the reaction scheme below:

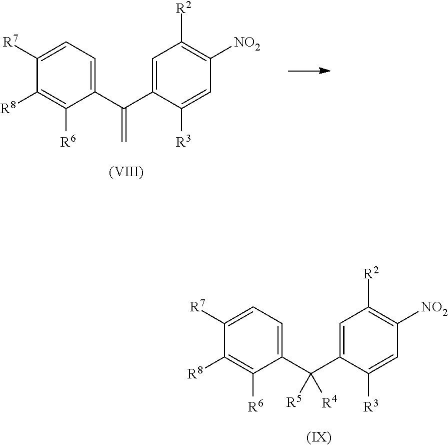

(e) reduction of nitrobenzene derivatives of formula (IX) to aniline derivatives of formula (V) according to the reaction scheme below:

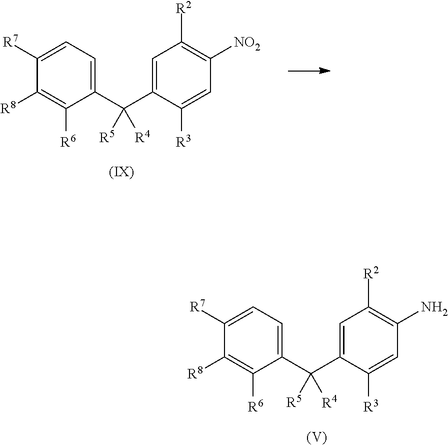

(f) reaction of anilines of formula (V) with aminoacetals to afford amidines of formula (I) according to the scheme below:

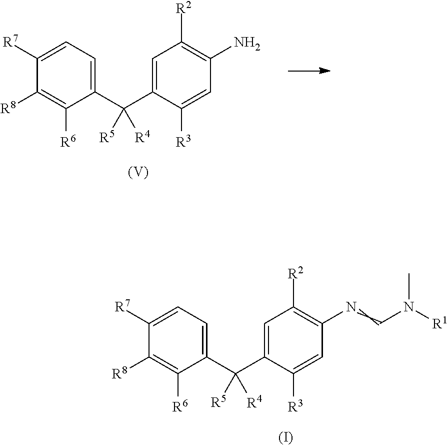

(g) reaction of an organometallic compound of formula (X) with aniline derivative of formula (II) to afford anilines of formula (V) according to the scheme below:

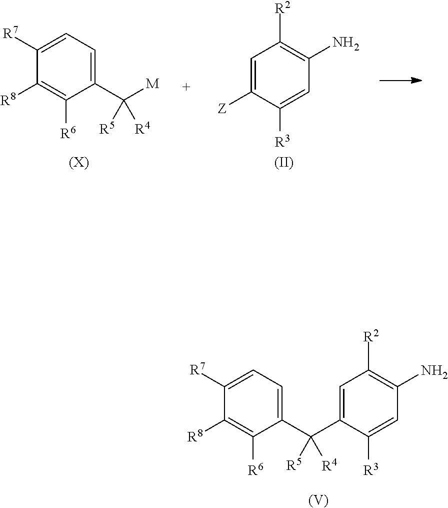

where in the above schemes
Z is selected from the group consisting of Cl, Br, I and OSO₂CF₃;
M is selected from the group consisting of MgZ and ZnZ;
R¹ to R⁸ have the above or below meanings.

A third subject matter of the invention is the use of the phenylamidines of the formula (I) according to the invention or of agrochemical formulations comprising these for controlling unwanted microorganisms, in particular for controlling phytopathogenic fungi. of a composition according to claim 8 for controlling phytopathogenic fungi.

A fourth subject matter of the present invention is an agrochemical formulation for controlling unwanted microorganisms, in particular for controlling phytopathogenic fungi, comprising at least one phenylamidines of the formula (I) according to the present invention.

A further subject matter of the invention relates to a method for controlling unwanted microorganisms, in particular for controlling phytopathogenic fungi, characterized in that the phenylamidines of the formula (I) according to the invention or agrochemical formulations comprising these are applied to the microorganisms and/or their habitat, in particular the phytopathogenic fungi and/or their habitat.

Moreover, the invention further relates to seed which has been treated with at least one compound of the formula (I).

The invention finally provides a method for protecting seed against unwanted microorganisms, in particular against phytopathogenic fungi, by using seed treated with at least one compound of the formula (I).

### General Definitions

In connection with the present invention, the term halogens (X) comprises, unless otherwise defined, those elements which are chosen from the group consisting of fluorine, chlorine, bromine and iodine, where fluorine, chlorine and bromine are preferably used, and fluorine and chlorine are particularly preferably used.

Optionally substituted groups can be mono- or polysubstituted, where in the case of polysubstitution the substituents can be identical or different.

In the definitions of the symbols given in the above formulae, collective terms were used, which are generally representative of the following substituents:

Hydrogen: Preferably, the definition of hydrogen encompasses also isotopes of hydrogen, preferably deuterium and tritium, more preferably deuterium.

Halogen: fluorine, chlorine, bromine and iodine and preferably fluorine, chlorine, bromine, and more preferably fluorine, chlorine.

Halomethyl: a methyl group, where some or all of the hydrogen atoms in these groups may be replaced by halogen atoms as specified above, for example (but not limited to) chloromethyl, bromomethyl, dichloromethyl, trichloromethyl, fluoromethyl, difluoromethyl, trifluoromethyl, chlorofluoromethyl, dichlorofluoromethyl, chlorodifluoromethyl.

Alkyl: saturated, straight-chain or branched hydrocarbyl radical having 1 to 8, preferably 1 to 6, and more preferably 1 to 4 carbon atoms, for example (but not limited to) C₁-C₆-alkyl such as methyl, ethyl, propyl (n-propyl), 1-methylethyl (iso-propyl), butyl (n-butyl), 1-methylpropyl (sec-butyl), 2-methylpropyl (iso-butyl), 1,1-dimethylethyl (tert-butyl), pentyl, 1-methylbutyl, 2-methylbutyl, 3-methylbutyl, 2,2-dimethylpropyl, 1-ethylpropyl, 1,1-dimethylpropyl, 1,2-dimethylpropyl, hexyl, 1-methylpentyl, 2-methylpentyl, 3-methylpentyl, 4-methylpentyl, 1,1-dimethylbutyl, 1,2-dimethylbutyl, 1,3-dimethylbutyl, 2,2-dimethylbutyl, 2,3-dimethylbutyl, 3,3-dimethylbutyl, 1-ethylbutyl, 2-ethylbutyl, 1,1,2-trimethylpropyl, 1,2,2-trimethylpropyl, 1-ethyl-1-methylpropyl and 1-ethyl-2-methylpropyl. Particularly, said group is a C₁-C₄-alkyl group, e.g. a methyl, ethyl, propyl, 1-methylethyl (isopropyl), butyl, 1-methylpropyl (sec-butyl), 2-methylpropyl (iso-butyl) or 1,1-dimethylethyl (tert-butyl) group.

This definition also applies to alkyl as part of a composite substituent, for example cycloalkylalkyl, hydroxyalkyl etc., unless defined elsewhere like, for example, alkylsulfanyl, alkylsulfinyl, alkylsulfonyl, haloalkyl or haloalkylsulfanyl.

Aryl: mono-, bi- or tricyclic aromatic or partially aromatic group having 6 to 14 carbon atoms, for example (but not limited to) phenyl, naphthyl, tetrahydronapthyl, indenyl and indanyl. The binding to the superordinate general structure can be carried out via any possible ring member of the aryl residue. Aryl is preferably selected from phenyl, 1-naphthyl and 2-naphthyl. Phenyl is particularly preferred.

Cycloalkyl: monocyclic, saturated hydrocarbyl groups having 3 to 7, preferably 3 to 6 carbon ring members, for example (but not limited to) cyclopropyl, cyclopentyl and cyclohexyl. This definition also applies to cycloalkyl as part of a composite substituent, for example cycloalkylalkyl etc., unless defined elsewhere. Cycloalkyl is particularly preferred cyclopropyl.

Heterocyclyl: three- to seven-membered, saturated or partially unsaturated heterocyclic group containing at least one, if appropriate up to four heteroatoms and/or heterogroups independently selected from the group consisting of N, O, P, S, S(═O) and S(═O)₂. The binding to the superordinate general structure can be carried out via a ring carbon atom or, if possible, via a ring nitrogen atom of the heterocyclic group. Saturated heterocyclic groups in this sense are for example (but not limited to) oxiranyl, aziridinyl, tetrahydrofuran-2-yl, tetrahydrofuran-3-yl, tetrahydrothien-2-yl, tetrahydrothien-3-yl, pyrrolidin-2-yl, pyrrolidin-3-yl, isoxazolidin-3-yl, isoxazolidin-4-yl, isoxazolidin-5-yl, isothiazolidin-3-yl, isothiazolidin-4-yl, isothiazolidin-5-yl, pyrazolidin-3-yl, pyrazolidin-4-yl, pyrazolidin-5-yl, oxazolidin-2-yl, oxazolidin-4-yl, oxazolidin-5-yl, thiazolidin-2-yl, thiazolidin-4-yl, thiazolidin-5-yl, imidazolidin-2-yl, imidazolidin-4-yl, 1,2,4-oxadiazolidin-3-yl, 1,2,4-oxadiazolidin-5-yl, 1,3,4-oxadiazolidin-2-yl, 1,2,4-thiadiazolidin-3-yl, 1,2,4-thiadiazolidin-5-yl, 1,3,4-thiadiazolidin-2-yl, 1,2,4-triazolidin-3-yl, 1,3,4-triazolidin-2-yl, piperidin-2-yl, piperidin-3-yl, piperidin-4-yl, 1,3-dioxan-5-yl, tetrahydropyran-2-yl, tetrahydropyran-4-yl, tetrahydrothien-2-yl, hexahydropyridazin-3-yl, hexahydropyridazin-4-yl, hexahydropyrimidin-2-yl, hexahydropyrimidin-4-yl, hexahydropyrimidin-5-yl, piperazin-2-yl, 1,3,5-hexahydrotriazin-2-yl and 1,2,4-hexahydrotriazin-3-yl. Partially unsaturated heterocyclic groups in this sense are for example (but not limited to) 2,3-dihydrofur-2-yl, 2,3-dihydrofur-3-yl, 2,4-dihydrofur-2-yl, 2,4-dihydrofur-3-yl, 2,3-dihydrothien-2-yl, 2,3-dihydrothien-3-yl, 2,4-dihydrothien-2-yl, 2,4-dihydrothien-3-yl, 2-pyrrolin-2-yl, 2-pyrrolin-3-yl, 3-pyrrolin-2-yl, 3-pyrrolin-3-yl, 2-isoxazolin-3-yl, 3-isoxazolin-3-yl, 4-isoxazolin-3-yl, 2-isoxazolin-4-yl, 3-isoxazolin-4-yl, 4-isoxazolin-4-yl, 2-isoxazolin-5-yl, 3-isoxazolin-5-yl, 4-isoxazolin-5-yl, 2-isothiazolin-3-yl, 3-isothiazolin-3-yl, 4-isothiazolin-3-yl, 2-isothiazolin-4-yl, 3-isothiazolin-4-yl, 4-isothiazolin-4-yl, 2-isothiazolin-5-yl, 3-isothiazolin-5-yl, 4-isothiazolin-5-yl, 2,3-dihydropyrazol-1-yl, 2,3-dihydropyrazol-2-yl, 2,3-dihydropyrazol-3-yl, 2,3-dihydropyrazol-4-yl, 2,3-dihydropyrazol-5-yl, 3,4-dihydropyrazol-1-yl, 3,4-dihydropyrazol-3-yl, 3,4-dihydropyrazol-4-yl, 3,4-dihydropyrazol-5-yl, 4,5-dihydropyrazol-1-yl, 4,5-dihydropyrazol-3-yl, 4,5-dihydropyrazol-4-yl, 4,5-dihydropyrazol-5-yl, 2,3-dihydrooxazol-2-yl, 2,3-dihydrooxazol-3-yl, 2,3-dihydrooxazol-4-yl, 2,3-dihydrooxazol-5-yl, 3,4-dihydrooxazol-2-yl, 3,4-dihydrooxazol-3-yl, 3,4-dihydrooxazol-4-yl, 3,4-dihydrooxazol-5-yl, 3,4-dihydrooxazol-2-yl, 3,4-dihydrooxazol-3-yl, 3,4-dihydrooxazol-4-yl. This definition also applies to heterocyclyl as part of a composite substituent, for example heterocyclylalkyl etc., unless defined elsewhere.

Not included are combinations which are contrary to natural laws and which the person skilled in the art, based on his expert knowledge, would thus have excluded

### Isomers

Depending on the nature of the substituents, the compound of the invention may be present in the form of different stereoisomers. These stereoisomers are, for example, enantiomers, diastereomers, atropisomers or geometric isomers. Accordingly, the invention encompasses both pure stereoisomers and any mixture of these isomers. Where a compound can be present in two or more tautomer forms in equilibrium, reference to the compound by means of one tautomeric description is to be considered to include all tautomer forms.

### Salts

Depending on the nature of the substituents, the compound of the invention may be present in the form of the free compound and/or an agriculturally acceptable salt thereof. The term “agriculturally acceptable salt” refers to a salt of the compound of the invention with acids or bases which are agriculturally acceptable.

The phenylamidines according to the invention are compounds of the formula (I)

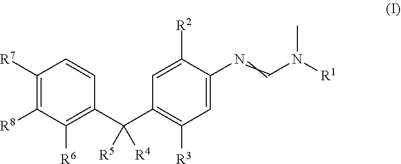

or their salts, N-oxides, metal complexes and their stereoisomers.

In the formula (I), the groups have the meanings defined below. The given definitions also apply to all intermediates:
R¹ is selected from the group consisting of C₁-C₈-alkyl, C₃-C₇-cycloalkyl which may be independently non-substituted or substituted by one or more group(s) selected from halogen or C₁-C₈-alkoxy;
R² and R³ are each independently selected from the group consisting of halogen, cyano, C₁-C₈-alkyl, C₃-C₇-cycloalkyl, —O—C₁-C₈-alkyl, C₂-C₈-alkenyl, C₂-C₈-alkynyl, —Si(R^{3a})(R^{3b})(R^{3c}), —C(O)—C₁-C₈-alkyl, —C(O)—C₃-C₇-cycloalkyl, —C(O)NH—C₁-C₈-alkyl, —C(O)N-di-C₁-C₈-alkyl, —C(O)O—C₁-C₈-alkyl, —S(O)ₙ—C₁-C₈-alkyl, —NH—C₁-C₈-alkyl, —N-di-C₁-C₈-alkyl, which may be independently non-substituted or substituted by one or more group(s) selected from halogen or C₁-C₈-alkoxy;
   wherein R^{3a}, R^{3b}, R^{3c} represent independently from each other phenyl or C₁-C₈-alkyl;
   n represents 0, 1 or 2;
R⁴, R⁵, R⁶, R⁷ and R⁸ are each independently selected from the group consisting of halogen, cyano, C₁-C₈-alkyl, C₃-C₇-cycloalkyl, —O—C₁-C₈-alkyl, C₂-C₈-alkenyl, C₂-C₈-alkynyl, —Si(R^{3a})(R^{3b})(R^{3c}), —C(O)—C₁-C₈-alkyl, —C(O)—C₃-C₇-cycloalkyl, —C(O)NH—C₁-C₈-alkyl, —C(O)N-di-C₁-C₈-alkyl, —C(O)O—C₁-C₈-alkyl, —S(O)ₙ—C₁-C₈-alkyl, —NH—C₁-C₈-alkyl, —N-di-C₁-C₈-alkyl, C₆-C₁₄-aryl, which may be independently non-substituted or substituted by one or more group(s) selected from halogen, methyl, halomethyl or C₁-C₈-alkoxy;
   wherein R^{3a}, R^{3b}, R^{3c} represent independently from each other phenyl or C₁-C₈-alkyl;
   n represents 0, 1 or 2;
or in which R⁴ and R⁵ can form, together with the atoms to which they are bonded or with additional atoms chosen from N, O, P and S, a 3- to 7-membered ring selected from the group consisting of cycloalkyl and heterocyclyl, which may optionally be substituted by one or more group(s) selected from halogen, and wherein R⁶, R⁷ and R⁸ are as defined above;
or in which R⁴ and R⁵ together can form a double bonded substituent ═CR⁹R¹⁰, wherein R⁹ and R¹⁰ are each independently selected from the group consisting of H, halogen, Me and Et, and wherein R⁶, R⁷ and R⁸ are as defined above.

In the formula (I), the groups have alternatively the meanings defined below. The given definitions also apply to all intermediates:
R¹ is selected from the group consisting of C₁-C₈-alkyl, C₃-C₇-cycloalkyl which may be independently non-substituted or substituted by one or more group(s) selected from halogen or C₁-C₈-alkoxy;
R² and R³ are each independently selected from the group consisting of halogen, cyano, C₁-C₈-alkyl, C₃-C₇-cycloalkyl, —O—C₁-C₈-alkyl, C₂-C₈-alkenyl, C₂-C₈-alkynyl, —Si(R^{3a})(R^{3b})(R^{3c}), —C(O)—C₁-C₈-alkyl, —C(O)—C₃-C₇-cycloalkyl, —C(O)NH—C₁-C₈-alkyl, —C(O)N-di-C₁-C₈-alkyl, —C(O)O—C₁-C₈-alkyl, —S(O)ₙ—C₁-C₈-alkyl, —NH—C₁-C₈-alkyl, —N-di-C₁-C₈-alkyl, which may be independently non-substituted or substituted by one or more group(s) selected from halogen or C₁-C₈-alkoxy;
   Wherein
   R^{3a}, R^{3b}, R^{3c} represent independently from each other phenyl or C₁-C₈-alkyl;
   n represents 0, 1 or 2;
R⁴, R⁵, R⁶and R⁷ are each independently selected from the group consisting of halogen, cyano, C₁-C₈-alkyl, C₃-C-₇-cycloalkyl, —O—C₁-C₈-alkyl, C₂-C₈-alkenyl, C₂-C₈-alkynyl, —Si(R^{3a})(R^{3b})(R^{3c}), —C(O)—C₁-C₈-alkyl, —C(O)—C₃-C₇-cycloalkyl, —C(O)NH—C₁-C₈-alkyl, —C(O)N-di-C₁-C₈-alkyl, —C(O)O—C₁-C₈-alkyl, —S(O)ₙ—C₁-C₈-alkyl, —NH—C₁-C₈-alkyl, —N-di-C₁-C₈-alkyl, which may be independently non-substituted or substituted by one or more group(s) selected from halogen or C₁-C₈-alkoxy;
   wherein R^{3a}, R^{3b}, R^{3c} represent independently from each other phenyl or C₁-C₈-alkyl;
   n represents 0, 1 or 2;
or in which R⁴ and R⁵ can form, together with the atoms to which they are bonded or with additional atoms chosen from N, O, P and S, a 3- to 7-membered ring; and
R⁸ is H.

In formula (I), the groups have the preferred meanings defined below. The definitions given as being preferred likewise apply to all intermediates:
R¹ is preferably selected from the group consisting of C₁-C₈-alkyl,
R² is preferably selected from the group consisting of halogen, cyano, C₁-C₈-alkyl which may be independently non-substituted or substituted by one or more group(s) selected from halogen or C₁-C₈-alkoxy;
R³ is preferably selected from the group consisting of halogen, cyano, C₁-C₈-alkyl which may be independently non-substituted or substituted by one or more group(s) selected from halogen or C₁-C₈-alkoxy;
R⁴ and R⁵ are preferably selected from the group consisting of H, halogen, cyano, C₁-C₈-alkyl which may be independently non-substituted or substituted by one or more group(s) selected from halogen or C₁-C₈-alkoxy;
or R⁴ and R⁵ can preferably form, together with the atoms to which they are bonded or with additional atoms chosen from N, O, P and S, a 3- to 7-membered ring selected from the group consisting of cycloalkyl and heterocyclyl, which may optionally be substituted by one or more group(s) selected from halogen;
or in which R⁴ and R⁵ together can preferably form a double bonded substituent ═CR⁹R¹⁰, wherein R⁹ and R¹⁰ are each independently selected from the group consisting of H, F, Cl, Me and Et;
R⁶, R⁷ and R⁸ are preferably independently selected from the group consisting of H, F, Cl, cyano Me, methoxy, phenyl and phenyl substituted by one or more substituents selected from the group consisting of halogen, Me and CF₃.

In the formula (I), the radicals have the particularly preferred meanings defined below. The definitions given as being particularly preferred likewise apply to all intermediates:
R¹ is particularly preferably selected from the group consisting of Me, Et, iPr;
R² is particularly preferably selected from the group consisting of Me, cyano, Cl, Br, I, CHF₂, CF₃;
R³ is particularly preferably selected from the group consisting of Me, cyano, F, Cl, Br, I;
R⁴ and R⁵ are each independently particularly preferably selected from the group consisting of H;
or R⁴ and R⁵ can particularly preferably form, together with the atoms to which they are bonded or with additional atoms chosen from N, O, P and S, a 3- to 7-membered ring selected from the group consisting of cycloalkyl and heterocyclyl, which may optionally be substituted by one or more group(s) selected from halogen;
or in which R⁴ and R⁵ together can particularly preferably form a double bonded substituent ═CH₂;
R⁶ is particularly preferably selected from the group consisting of H, Me, Cyano, F;
R⁷ and R⁸ are particularly preferably H;

In the formula (I), the radicals have alternatively the particularly preferred meanings defined below. The definitions given as being particularly preferred likewise apply to all intermediates:
R¹ is particularly preferably selected from the group consisting of C₁-C₈-alkyl,
R² is particularly preferably selected from the group consisting of halogen, cyano, C₁-C₈-alkyl which may be independently non-substituted or substituted by one or more group(s) selected from halogen;
R³ is particularly preferably selected from the group consisting of halogen, cyano, C₁-C₈-alkyl which may be independently non-substituted or substituted by one or more group(s) selected from halogen;
R⁴ and R⁵ are particularly preferably selected from the group consisting of H, halogen, cyano, C₁-C₈-alkyl which may be independently non-substituted or substituted by one or more group(s) selected from halogen;
or R⁴ and R⁵ can particularly preferably form, together with the atom to which they are bonded a 3- to 6-membered ring selected from the group consisting of cycloalkyl, which may optionally be substituted by one or more group(s) selected from halogen;
or in which R⁴ and R⁵ together can particularly preferably form a double bonded substituent ═CR⁹R¹⁰, wherein R⁹ and R¹⁰ are each independently selected from the group consisting of hydrogen, F, Cl, Me and Et;
R⁶, R⁷ and R⁸ are particularly preferably independently selected from the group consisting of H, F, Cl, cyano, Me, methoxy and phenyl.

In the formula (I), the radicals have the more particularly preferred meanings defined below. The definitions given as being more particularly preferred likewise apply to all intermediates:
R¹ is more particularly preferably selected from the group consisting of Me, Et, iPr;
R² is more particularly preferably selected from the group consisting of Me, cyano, Cl, Br, I, CHF₂, CF₃;
R³ is more particularly preferably selected from the group consisting of Me, iPr, Cyano, F, Cl, Br, I;
R⁴ and R⁵ are more particularly preferably each independently selected from the group consisting of H and Me;
or R⁴ and R⁵ can more particularly preferably form, together with the atom to which they are bonded a cyclopropyl, which may optionally be substituted by one or more group(s) selected from the group consisting of F, Cl and Br;
or in which R⁴ and R⁵ together can more particularly preferably form a double bonded substituent ═CH₂;
R⁶ is more particularly preferably selected from the group consisting of H, Me, cyano, F, Cl, methoxy and phenyl;
R⁷ is more particularly preferably selected from the group consisting of H and F, and
R⁸ is more particularly preferably selected from the group consisting of H and F.

In the formula (I), the radicals have the even more particularly preferred meanings defined below. The definitions given as being even more particularly preferred likewise apply to all intermediates:
R¹ is even more particularly preferably selected from the group consisting of Et and iPr;
R² is even more particularly preferably selected from the group consisting of Me and Cl;
R³ is even more particularly preferably selected from the group consisting of Me, F and Cl;
R⁴ is even more particularly preferably selected from the group consisting of H and Me, and
R⁵ is even more particularly preferably H;
or R⁴ and R⁵ can even more particularly preferably form, together with the atom to which they are bonded a cyclopropyl, which may optionally be substituted by one or two F;
or in which R⁴ and R⁵ together can even more particularly preferably form a double bonded substituent ═CH₂;
R⁶ is even more particularly preferably selected from the group consisting of H, Me, cyano, F and Cl;
R⁷ is even more particularly preferably selected from the group consisting of H and F, and
R⁸ is even more particularly preferably selected from the group consisting of H and F.

Compounds in connection with the present invention are preferably compounds of formula (I) selected from the group consisting of Table 1:

TABLE 1
Preferred phenylamidines according to the present invention;
CN = cyano; iPr = isopropyl;

| Example | R¹ | R² | R³ | R⁴ | R⁵ | R⁶ | R⁷ | R⁸ |
|---|---|---|---|---|---|---|---|---|
| 1 | Et | Cl | Me | H | H | CN | H | H |
| 2 | Et | Cl | Me | H | H | F | H | H |
| 3 | Et | Cl | Me | H | H | H | H | H |
| 4 | Et | Me | Cl | H | H | CN | H | H |
| 5 | Et | Me | Cl | H | H | F | H | H |
| 6 | Et | Me | Cl | H | H | H | H | H |
| 7 | Et | Me | Me | H | H | Cl | H | H |
| 8 | Et | Me | Me | H | H | CN | H | H |
| 9 | Et | Me | Me | H | H | F | F | H |
| 10 | Et | Me | Me | H | H | F | H | H |
| 11 | Et | Me | Me | H | H | H | H | H |
| 12 | iPr | Me | Me | H | H | F | F | H |
| 13 | Et | Me | Me | —CH₂—CH₂— | | H | H | H |
| 14 | Et | Me | Me | —CF₂—CH₂— | | H | H | H |
| 15 | Et | Me | Me | —CCl₂—CH₂— | | H | H | H |
| 16 | Et | Me | Me | —CBr₂—CH₂— | | H | H | H |
| 18 | Et | Me | Cl | Me | H | H | H | H |
| 19 | Et | Me | Cl | ═CH₂ | | H | H | H |
| 20 | Et | Me | F | H | H | H | H | H |
| 21 | Et | Cl | Me | H | H | F | F | H |
| 22 | Et | Me | Cl | H | H | F | F | H |
| 23 | Et | Me | Cl | H | H | Me | H | H |
| 24 | Et | Me | Cl | H | H | F | H | F |
| 25 | Et | Me | Cl | H | H | Ph | H | H |
| 26 | Et | Cl | Me | H | H | OMe | H | H |
| 27 | Et | Cl | Me | Me | H | H | H | H |
| 28 | Et | Cl | Me | H | H | Cl | H | H |
| 29 | Et | Me | Cl | H | H | OMe | H | H |
| 30 | Et | Me | Cl | H | H | Cl | H | H |
| 31 | Et | Cl | Me | ═CH₂ | | H | H | H |
| 32 | Et | Me | F | H | H | OMe | H | H |
| 33 | Et | Me | F | H | H | F | H | H |
| 34 | Et | Me | F | Me | H | H | H | H |
| 35 | Et | Me | F | H | H | F | F | H |
| 36 | Et | Me | iPr | H | H | F | H | H |
| 37 | Et | Me | iPr | H | H | CN | H | H |
| 38 | Et | Me | cPr | H | H | F | H | H |
| 39 | Et | Me | cPr | H | H | CN | H | H |
| 40 | iPr | Me | iPr | H | H | F | H | H |
| 41 | iPr | Me | iPr | H | H | CN | H | H |
| 42 | iPr | Me | cPr | H | H | F | H | H |
| 43 | iPr | Me | cPr | H | H | CN | H | H |
| 44 | Et | F | Me | H | H | H | H | H |
| 45 | Et | Cl | Me | H | H | H | H | H |
| 46 | iPr | Cl | Me | H | H | H | H | H |
| 47 | Et | CN | Me | H | H | H | H | H |
| 48 | Et | CF₃ | Me | H | H | H | H | H |
| 49 | Et | CHF₂ | Me | H | H | H | H | H |
| 50 | Et | Br | Me | H | H | H | H | H |
| 51 | Et | I | Me | H | H | H | H | H |
| 52 | Et | cPr | Me | H | H | H | H | H |
| 53 | Et | iPr | Me | H | H | H | H | H |
| 54 | Et | C≡CH | Me | H | H | H | H | H |

The compounds of the formula (I) carry amidine groups which induce basic properties. Thus, these compounds can be reacted with acids to give salts.

Examples of inorganic acids are hydrohalic acids, such as hydrogen fluoride, hydrogen chloride, hydrogen bromide and hydrogen iodide, sulphuric acid, phosphoric acid and nitric acid, and acidic salts, such as NaHSO₄ and KHSO₄.

As organic acids come, for example, formic acid, carbonic acid and alkanoic acids such as acetic acid, trifluoroacetic acid, trichloroacetic acid and propionic acid, and also glycolic acid, thiocyanic acid, lactic acid, succinic acid, citric acid, benzoic acid, cinnamic acid, oxalic acid, saturated or mono- or diunsaturated C₆-C₂₀ fatty acids, alkylsulphonic acids (sulphonic acids having straight-chain or branched alkyl radicals having 1 to 20 carbon atoms), arylsulphonic acids or aryldisulphonic acids (aromatic radicals, such as phenyl and naphthyl, which bear one or two sulphonic acid groups), alkylphosphonic acids (phosphonic acids having straight-chain or branched alkyl radicals having 1 to 20 carbon atoms), arylphosphonic acids or aryldiphosphonic acids (aromatic radicals, such as phenyl and naphthyl, which bear one or two phosphonic acid radicals), where the alkyl and aryl radicals may bear further substituents, for example p-toluenesulphonic acid, salicylic acid, p-aminosalicylic acid, 2-phenoxybenzoic acid, 2-acetoxybenzoic acid, etc.

Useful metal ions are especially the ions of the elements of the second main group, especially calcium and magnesium, of the third and fourth main group, especially aluminum and tin, and also of the first to eighth transition groups, especially manganese, iron, cobalt, nickel, copper, zinc and others. Particular preference is given to the metal ions of the elements of the fourth period. The metals may be present in the different valences that they can assume.

### Preparation of the Phenylamidines of the Formula (I) According to the Invention

The phenylamidines of the formula (I) according to the invention can be obtained by the process shown in scheme (I) below:

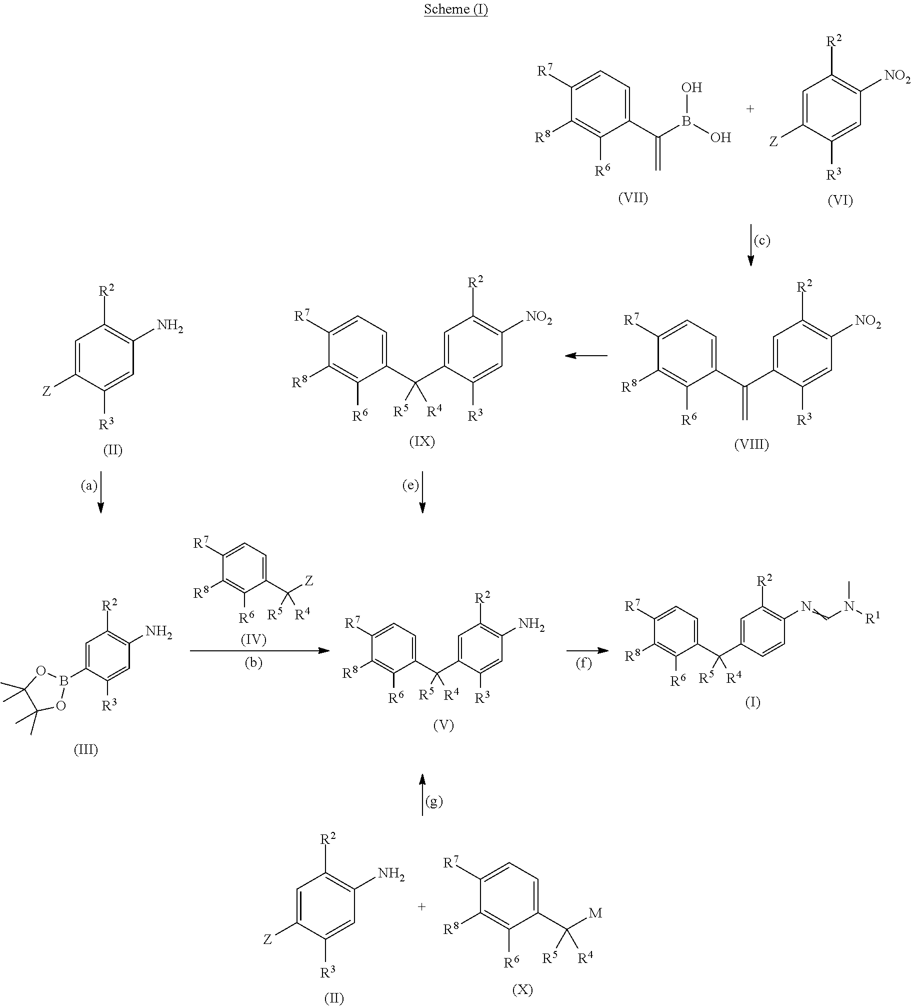

where in the above schemes
Z is selected from the group consisting of Cl, Br, I and OSO₂CF₃;
M is selected from the group consisting of MgZ and ZnZ;
R¹ to R⁸ have the above meanings;

### Step (a)

In one embodiment according to the invention, anilines derivatives of formula (II) are reacted with bispinacoldiboron to give the corresponding boronic esters of formula (III) in accordance with the reaction scheme below:

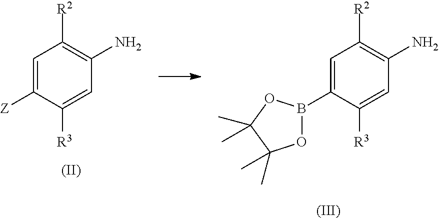

Suitable groups (Z) are all substituents having sufficient reactivity under the prevailing reaction conditions. Examples of suitable (Z) groups to be mentioned are halogens and triflate.

Such couplings can be performed by methods described in the literature (see e.g “Palladium in heterocyclic chemistry”, Pergamon Press, 2000; 1^{st} edition, J. Li & G. Gribble) via a coupling reaction, optionally in the presence of a catalyst, preferably a transition metal catalyst, such as copper salts, palladium salts or complexes for example palladium (II) chloride, palladium (II) acetate, tetrakis(triphenylphosphine) palladium(0), bis-(triphenylphosphine) palladium dichloride (II), tris(dibenzylideneacetone) dipalladium(0), bis(dibenzylideneacetone) palladium(0), or 1,1′-bis(diphenylphosphino)ferrocene-palladium (II) chloride. As an alternative the palladium complex is directly generated in the reaction mixture by separately adding to the reaction mixture a palladium salt and a complex ligand such as a phosphine, for example triethylphosphine, tri-tert-butylphosphine, tricyclohexylphosphine, 2-(dicyclohexylphosphine)biphenyl, 2-(di-tert-butylphosphin)biphenyl, 2-(dicyclohexylphosphine)-2′-(N,N-dimethylamino)-biphenyl, triphenylphosphine, tris-(o-tolyl)phosphine, sodium 3-(diphenylphosphino)benzolsulfonate, tris-2-(methoxyphenyl)phosphine, 2,2′-bis-(diphenylphosphine)-1,1′-binaphthyl, 1,4-bis-(diphenylphosphine)butane, 1,2-bis-(diphenylphosphine)ethane, 1,4-bis-(dicyclohexylphosphine)butane, 1,2-bis-(dicyclohexylphosphine)ethane, 2-(dicyclohexylphosphine)-2′-(N,N-dimethylamino)-biphenyl, bis(diphenylphosphino)ferrocene, tris-(2,4-tert-butylphenyl)-phosphite, (R)-(−)-1-[(S)-2-(diphenylphosphino)ferrocenyl]ethyldi-tert-butylphosphine, (S)-(+)-1-[(R)-2-(diphenylphosphino)ferrocenyl]ethyldicyclohexylphosphine, (R)-(−)-1-[(S)-2-(diphenylphosphino)ferrocenyl]ethyldicyclohexylphosphine, (S)-(+)-1-[(R)-2-(diphenylphosphino)ferrocenyl]ethyldi-t-butylphosphine.

Such coupling reactions are optionally performed in the presence of a base such as an inorganic or an organic base; preferably an alkaline earth metal or alkali metal hydride, hydroxide, amide, alcoholate, acetate, carbonate or hydrogen carbonate, such as sodium hydride, sodium amide, lithiium diisopropylamide, sodium methanolate, sodium ethanolate, potassium tert-butanolate, sodium acetate, potassium acetate, calcium acetate, sodium hydroxide, potassium hydroxide, sodium carbonate, potassium carbonate, potassium bicarbonate, sodium bicarbonate, cesium carbonate or ammonium carbonate; and also tertiary amine, such as trimethylamine, triethylamine (TEA), tributylamine, N,N-dimethylaniline, N,N-dimethyl-benzylamine, N,N-diisopropyl-ethylamine (DIPEA), pyridine, N-methylpiperidine, N-methylmorpholine, N,N-dimethylaminopyridine, diazabicyclooctane (DABCO), diazabicyclononene (DBN) or diazabicycloundecene (DBU).

The reaction can be carried out neat or in a solvent; preferably, the reaction is carried out in a solvent selected from standard solvents which are inert under the prevailing reaction conditions.

Preference is given to aliphatic, alicyclic or aromatic hydrocarbons, such as, for example, petroleum ether, hexane, heptane, cyclohexane, methylcyclohexane, benzene, toluene, xylene or decalin; halogenated hydrocarbons, such as, for example, chlorobenzene, dichlorobenzene, dichloromethane, chloroform, carbon tetrachloride, dichloroethane or trichloroethane; ethers, such as, for example, diethyl ether, diisopropyl ether, methyl tert-butyl ether (MTBE), methyl tert-amyl ether, dioxane, tetrahydrofuran, 1,2-dimethoxyethane, 1,2-diethoxyethane or anisole; nitriles, such as, for example, acetonitrile, propionitrile, n- or isobutyronitrile or benzonitrile; amides, such as, for example, N,N-dimethylformamide (DMF), N,N-dimethylacetamide, N-methylformanilide, N-methylpyrrolidone (NMP) or hexamethylenephosphoric triamide; or mixtures of these with water, and also pure water.

The reaction can be carried out under reduced pressure, at atmospheric pressure or under superatmospheric pressure and at temperatures of from −20 to 200° C.; preferably, the reaction is carried out at atmospheric pressure and temperatures of from 50 to 150° C.

The aniline derivatives of the formula (II) are commercially available or can be prepared from commercially available precursors by methods described in the literature.

### Step (b)

In an alternative embodiment according to the invention, boronic ester derivatives of the formula (III) can be reacted with benzyl derivatives of formula (IV) to afford the anilines derivatives of formula (V) in accordance with the reaction scheme below:

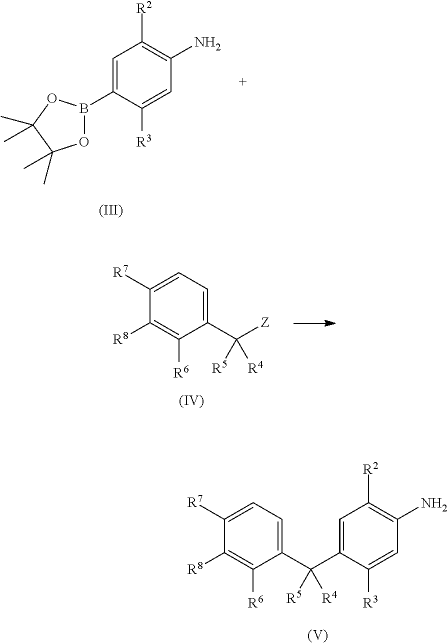

Suitable groups (Z) are all substituents having sufficient reactivity under the prevailing reaction conditions. Examples of suitable (Z) groups to be mentioned are halogens and triflate.

The reaction can be carried out in conditions similar to those described in step (a)

Benzyl derivatives of formula (IV) are are commercially available or can be prepared from commercially available precursors by methods described in the literature.

### Step (c)

The nitrophenyl derivatives of formula (VI) can be reacted with alkenyl boronic acid derivatives of formula (VII) in accordance with the reaction scheme below to give the alkenyl derivatives of the formula (VIII):

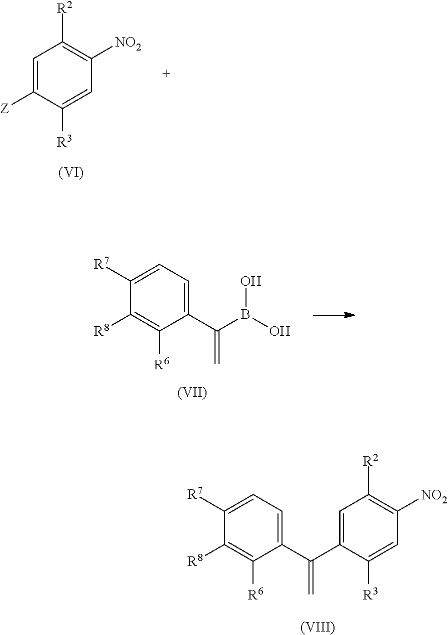

Suitable groups (Z) are all substituents having sufficient reactivity under the prevailing reaction conditions. Examples of suitable (Z) groups to be mentioned are halogens and triflate.

The reaction can be carried out in conditions similar to those described in step (a)

Nitrobenzene derivatives of formula (VI) and Alkenyl derivatives of formula (VII) are commercially available or can be prepared from commercially available precursors by methods described in the literature.

### Step (d)

The alkenyl derivatives of formula (VIII) can be transformed into cyclopropyl derivatives of formula (IX) according to the reaction scheme below:

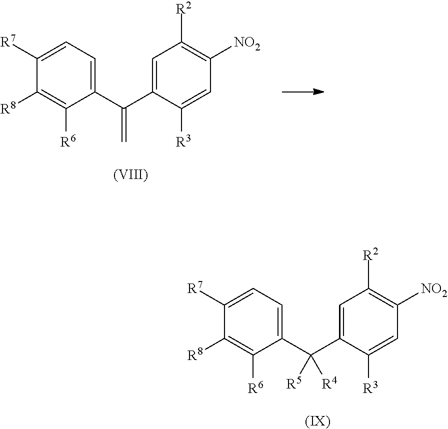

The reactions can be performed according to methods described in the literature and can involve the generation of a carbene intermediate. Suitable conditions to perform this reaction comprise the use of a haloform in the presence of a base such as an inorganic or an organic base; preferably an alkaline earth metal or alkali metal hydride, hydroxide, amide, alcoholate, acetate, carbonate or hydrogen carbonate, such as sodium hydride, sodium amide, lithiium diisopropylamide, sodium methanolate, sodium ethanolate, potassium tert-butanolate, sodium acetate, potassium acetate, calcium acetate, sodium hydroxide, potassium hydroxide, sodium carbonate, potassium carbonate, potassium bicarbonate, sodium bicarbonate, cesium carbonate or ammonium carbonate; and also tertiary amine, such as trimethylamine, triethylamine (TEA), tributylamine, N,N-dimethylaniline, N,N-dimethyl-benzylamine, N,N-diisopropyl-ethylamine (DIPEA), pyridine, N-methylpiperidine, N-methylmorpholine, N,N-dimethylaminopyridine, diazabicyclooctane (DABCO), diazabicyclononene (DBN) or diazabicycloundecene (DBU).

Alternatively, the reaction can be performed using an haloacetate salt (eg BrCF₂CO₂Na) or the corresponding haloacetic acid in the presence of a suitable base as described above.

Alternatively, the reaction can be performed using a dihalomethane (eg diiodomethane) in the presence of transition metal or transition metal derivative (eg diethyl zinc).

The reaction can be carried out neat or in a solvent; preferably, the reaction is carried out in a solvent selected from standard solvents which are inert under the prevailing reaction conditions.

Preference is given to aliphatic, alicyclic or aromatic hydrocarbons, such as, for example, petroleum ether, hexane, heptane, cyclohexane, methylcyclohexane, benzene, toluene, xylene or decalin; halogenated hydrocarbons, such as, for example, chlorobenzene, dichlorobenzene, dichloromethane, chloroform, carbon tetrachloride, dichloroethane or trichloroethane; ethers, such as, for example, diethyl ether, diisopropyl ether, methyl tert-butyl ether (MTBE), methyl tert-amyl ether, dioxane, tetrahydrofuran, 1,2-dimethoxyethane, 1,2-diethoxyethane or anisole; nitriles, such as, for example, acetonitrile, propionitrile, n- or isobutyronitrile or benzonitrile; amides, such as, for example, N,N-dimethylformamide (DMF), N,N-dimethylacetamide, N-methylformanilide, N-methylpyrrolidone (NMP) or hexamethylenephosphoric triamide; or mixtures of these with water, and also pure water.

The reaction can be carried out under reduced pressure, at atmospheric pressure or under superatmospheric pressure and at temperatures of from −20 to 200° C.; preferably, the reaction is carried out at atmospheric pressure and temperatures of from 0 to 150° C.

### Step (e)

The nitrophenyl derivatives of formula (VIII) can be reduced to aniline derivatives of formula (V) in accordance with the reaction scheme below:

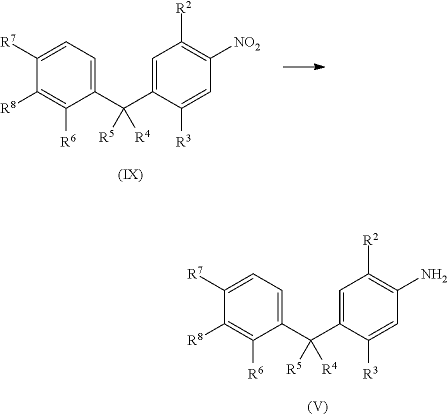

The reduction according to step (e) can be carried out by any methods for reducing nitro groups described in the prior art.

Preferably, the reduction is carried out using tin chloride as described in WO2000/46184. However, alternatively, the reduction can also be carried out by using iron in the presence of hydrochloric acid or hydrogen gas, if appropriate in the presence of suitable hydrogenation catalysts, such as, for example, Raney nickel or Pd/C. The reaction conditions have already been described in the prior art and are familiar to the person skilled in the art.

If the reduction is carried out in the liquid phase, the reaction should take place in a solvent inert to the prevailing reaction conditions. One such solvent is, for example, toluene, methanol, or ethanol.

### Step (f)

The conversion of the anilines of the formula (V) into the amidines of formula (I) can be carried out as shown below:

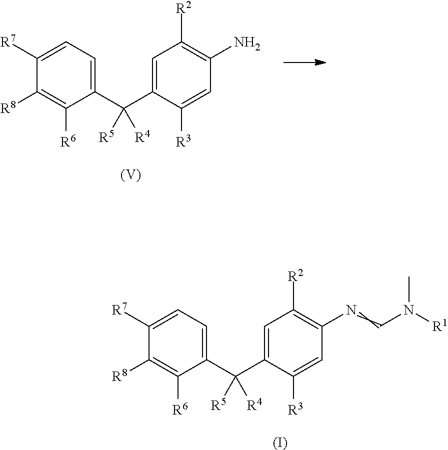

The reaction according to step (f) is preferably carried out in the presence of an aminoacetal of formula MeR₁NCH(OMe)₂ and preferably in the absence of a base or an acid.

The reaction is preferably carried out in a solvent selected from standard solvents which are inert under the prevailing reaction conditions. Preference is given to aliphatic, alicyclic or aromatic hydrocarbons, such as, for example, petroleum ether, hexane, heptane, cyclohexane, methylcyclohexane, benzene, toluene, xylene or decalin; halogenated hydrocarbons, such as, for example, chlorobenzene, dichlorobenzene, dichloromethane, chloroform, carbon tetrachloride, dichloroethane or trichloroethane; ethers, such as, for example, diethyl ether, diisopropyl ether, methyl tert-butyl ether (MTBE), methyl tert-amyl ether, dioxane, tetrahydrofuran, 1,2-dimethoxyethane, 1,2-diethoxyethane or anisole; nitriles, such as, for example, acetonitrile, propionitrile, n- or isobutyronitrile or benzonitrile; amides, such as, for example, N,N-dimethylformamide (DMF), N,N-dimethylacetamide, N-methylformanilide, N-methylpyrrolidone (NMP) or hexamethylenephosphoric triamide; esters, such as, for example, methyl acetate or ethyl acetate; sulfoxides, such as, for example, dimethyl sulfoxide (DMSO); sulfones, such as, for example, sulfolane; alcohols, such as, for example, methanol, ethanol, n- or isopropanol, n-, iso-, sec- or tert-butanol, ethanediol, propane-1,2-diol, ethoxyethanol, methoxyethanol, diethylene glycol monomethyl ether, diethylene glycol monoethyl ether or mixtures of these.

### Step (g)

The conversion of organometallic compounds of formula (X) into the anilines of formula (V) can be carried out as shown below:

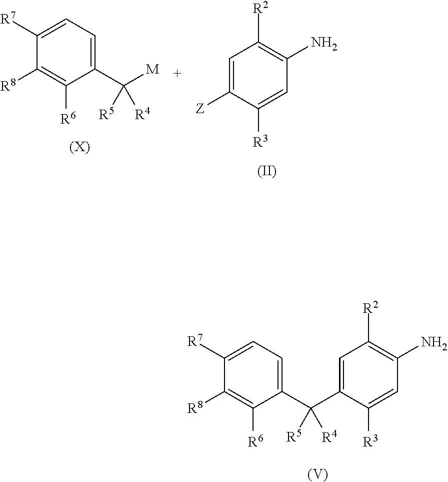

Suitable groups (Z) are all substituents having sufficient reactivity under the prevailing reaction conditions. Examples of suitable (Z) groups to be mentioned are halogens and triflate.

Suitable groups (M) are all substituents having sufficient reactivity under the prevailing reaction conditions. Examples of suitable (M) groups to be mentioned are MgZ and ZnZ.

The reaction can be carried out in conditions similar to those described in step (a)

Organometallic compounds of formula (X) are commercially available or can be prepared from commercially available precursors by methods described in the literature.

In the above schemes
Z is selected from the group consisting of Cl, Br, I and OSO₂CF₃;
M is selected from the group consisting of MgZ and ZnZ;
R¹to R⁸ have the meanings as defined herein.

### Compositions/Formulations

The present invention further relates to a composition, in particular a composition for controlling unwanted microorganisms, in particular phytopathogenic fungi. The compositions may be applied to the microorganisms, in particular phytopathogenic fungi and/or in their habitat. The term “compositions” encompasses agrochemical formulations.

The composition typically comprises at least one compound of formula (I) and at least one agriculturally suitable auxiliary, e.g. carrier(s) and/or surfactant(s).

A carrier is a solid or liquid, natural or synthetic, organic or inorganic substance that is generally inert. The carrier generally improves the application of the compounds, for instance, to plants, plants parts or seeds. Examples of suitable solid carriers include, but are not limited to, ammonium salts, natural rock flours, such as kaolins, clays, talc, chalk, quartz, attapulgite, montmorillonite and diatomaceous earth, and synthetic rock flours, such as finely divided silica, alumina and silicates. Examples of typically useful solid carriers for preparing granules include, but are not limited to crushed and fractionated natural rocks such as calcite, marble, pumice, sepiolite and dolomite, synthetic granules of inorganic and organic flours and granules of organic material such as paper, sawdust, coconut shells, maize cobs and tobacco stalks. Examples of suitable liquid carriers include, but are not limited to, water, organic solvents and combinations thereof. Examples of suitable solvents include polar and nonpolar organic chemical liquids, for example from the classes of aromatic and nonaromatic hydrocarbons (such as cyclohexane, paraffins, alkylbenzenes, xylene, toluene alkylnaphthalenes, chlorinated aromatics or chlorinated aliphatic hydrocarbons such as chlorobenzenes, chloroethylenes or methylene chloride), alcohols and polyols (which may optionally also be substituted, etherified and/or esterified, such as butanol or glycol), ketones (such as acetone, methyl ethyl ketone, methyl isobutyl ketone or cyclohexanone), esters (including fats and oils) and (poly)ethers, unsubstituted and substituted amines, amides (such as dimethylformamide), lactams (such as N-alkylpyrrolidones) and lactones, sulphones and sulphoxides (such as dimethyl sulphoxide). The carrier may also be a liquefied gaseous extender, i.e. liquid which is gaseous at standard temperature and under standard pressure, for example aerosol propellants such as halohydrocarbons, butane, propane, nitrogen and carbon dioxide.

The surfactant can be an ionic (cationic or anionic) or non-ionic surfactant, such as ionic or non-ionic emulsifier(s), foam former(s), dispersant(s), wetting agent(s) and any mixtures thereof. Examples of suitable surfactants include, but are not limited to, salts of polyacrylic acid, salts of lignosulphonic acid, salts of phenolsulphonic acid or naphthalenesulphonic acid, polycondensates of ethylene and/or propylene oxide with fatty alcohols, fatty acids or fatty amines (polyoxyethylene fatty acid esters, polyoxyethylene fatty alcohol ethers, for example alkylaryl polyglycol ethers), substituted phenols (preferably alkylphenols or arylphenols), salts of sulphosuccinic esters, taurine derivatives (preferably alkyl taurates), phosphoric esters of polyethoxylated alcohols or phenols, fatty esters of polyols and derivatives of compounds containing sulphates, sulphonates, phosphates (for example, alkylsulphonates, alkyl sulphates, arylsulphonates) and protein hydrolysates, lignosulphite waste liquors and methylcellulose. A surfactant is typically used when the compound of the formula (I) and/or the carrier is insoluble in water and the application is made with water. Then, the amount of surfactants typically ranges from 5 to 40% by weight of the composition.

Further examples of suitable auxiliaries include water repellents, siccatives, binders (adhesive, tackifier, fixing agent, such as carboxymethylcellulose, natural and synthetic polymers in the form of powders, granules or latices, such as gum arabic, polyvinyl alcohol and polyvinyl acetate, natural phospholipids such as cephalins and lecithins and synthetic phospholipids, polyvinylpyrrolidone, polyvinyl acetate, polyvinyl alcohol and tylose), thickeners, stabilizers (e.g. cold stabilizers, preservatives, antioxidants, light stabilizers, or other agents which improve chemical and/or physical stability), dyes or pigments (such as inorganic pigments, e.g. iron oxide, titanium oxide and Prussian Blue; organic dyes, e.g. alizarin, azo and metal phthalocyanine dyes), antifoams (e.g. silicone antifoams and magnesium stearate), preservatives (e.g. dichlorophene and benzyl alcohol hemiformal), secondary thickeners (cellulose derivatives, acrylic acid derivatives, xanthan, modified clays and finely divided silica), stickers, gibberellins and processing auxiliaries, mineral and vegetable oils, perfumes, waxes, nutrients (including trace nutrients, such as salts of iron, manganese, boron, copper, cobalt, molybdenum and zinc), protective colloids, thixotropic substances, penetrants, sequestering agents and complex formers.

The choice of the auxiliaries is related to the intended mode of application of the compound of the formula (I) and/or on the physical properties. Furthermore, the auxiliaries may be chosen to impart particular properties (technical, physical and/or biological properties) to the compositions or use forms prepared therefrom. The choice of auxiliaries may allow customizing the compositions to specific needs.

The composition of the invention may be in any customary form, such as solutions (e.g aqueous solutions), emulsions, wettable powders, water- and oil-based suspensions, powders, dusts, pastes, soluble powders, soluble granules, granules for broadcasting, suspoemulsion concentrates, natural or synthetic products impregnated with the compound of the invention, fertilizers and also microencapsulations in polymeric substances. The compound of the invention may be present in a suspended, emulsified or dissolved form.

The composition of the invention may be provided to the end user as ready-for-use formulation, i.e. the compositions can be directly applied to the plants or seeds by a suitable device, such as a spraying or dusting device. Alternatively, the compositions may be provided to the end user in the form of concentrates which have to be diluted, preferably with water, prior to use.

The composition of the invention can be prepared in conventional manners, for example by mixing the compound of the invention with one or more suitable auxiliaries, such as disclosed herein above.

The compositions according to the invention contain generally from 0.01 to 99% by weight, from 0.05 to 98% by weight, preferably from 0.1 to 95% by weight, more preferably from 0.5 to 90% by weight, most preferably from 10 to 70% by weight of the compound of the invention.

### Mixtures/Combinations

The compound and the composition of the invention can be mixed with other active ingredients like fungicides, bactericides, acaricides, nematicides, insecticides, herbicides, fertilizers, growth regulators, safeners or semiochemicals. This may allow to broaden the activity spectrum or to prevent development of resistance. Examples of known fungicides, insecticides, acaricides, nematicides and bactericides are disclosed in the Pesticide Manual, 17th Edition.

Examples of especially preferred fungicides which could be mixed with the compound and the composition of the invention are:

1) Inhibitors of the ergosterol biosynthesis, for example (1.001) cyproconazole, (1.002) difenoconazole, (1.003) epoxiconazole, (1.004) fenhexamid, (1.005) fenpropidin, (1.006) fenpropimorph, (1.007) fenpyrazamine, (1.008) fluquinconazole, (1.009) flutriafol, (1.010) imazalil, (1.011) imazalil sulfate, (1.012) ipconazole, (1.013) metconazole, (1.014) myclobutanil, (1.015) paclobutrazol, (1.016) prochloraz, (1.017) propiconazole, (1.018) prothioconazole, (1.019) Pyrisoxazole, (1.020) spiroxamine, (1.021) tebuconazole, (1.022) tetraconazole, (1.023) triadimenol, (1.024) tridemorph, (1.025) triticonazole, (1.026) (1R,2S,5S)-5-(4-chlorobenzyl)-2-(chloromethyl)-2-methyl-1-(1H-1,2,4-triazol-1-ylmethyl)cyclopentanol, (1.027) (1S,2R,5R)-5-(4-chlorobenzyl)-2-(chloromethyl)-2-methyl-1-(1H-1,2,4-triazol-1-ylmethyl)cyclopentanol, (1.028) (2R)-2-(1-chlorocyclopropyl)-4-[(1R)-2,2-dichloro-cyclopropyl]-1-(1H-1,2,4-triazol-1-yl)butan-2-ol, (1.029) (2R)-2-(1-chlorocyclopropyl)-4-[(1S)-2,2-dichlorocyclopropyl]-1-(1H-1,2,4-triazol-1-yl)butan-2-ol, (1.030) (2R)-2-[4-(4-chlorophenoxy)-2-(trifluoromethyl)phenyl]-1-(1H-1,2,4-triazol-1-yl)propan-2-ol, (1.031) (2S)-2-(1-chlorocyclopropyl)-4-[(1R)-2,2-dichlorocyclopropyl]-1-(1H-1,2,4-triazol-1-yl)butan-2-ol, (1.032) (2S)-2-(1-chlorocyclopropyl)-4-[(1S)-2,2-dichlorocyclopropyl]-1-(1H-1,2,4-triazol-1-yl)butan-2-ol, (1.033) (2S)-2-[4-(4-chlorophenoxy)-2-(trifluoromethyl)phenyl]-1-(1H-1,2,4-triazol-1-yl)propan-2-ol, (1.034) (R)-[3-(4-chloro-2-fluorophenyl)-5-(2,4-difluorophenyl)-1,2-oxazol-4-yl](pyridin-3-yl)methanol, (1.035) (S)-[3-(4-chloro-2-fluorophenyl)-5-(2,4-difluorophenyl)-1,2-oxazol-4-yl](pyridin-3-yl)methanol, (1.036) [3-(4-chloro-2-fluorophenyl)-5-(2,4-difluorophenyl)-1,2-oxazol-4-yl](pyridin-3-yl)methanol, (1.037) 1-({(2R,4S)-2-[2-chloro-4-(4-chlorophenoxy)phenyl]-4-methyl-1,3-dioxolan-2-yl}methyl)-1H-1,2,4-triazole, (1.038) 1-({(2S,4S)-2-[2-chloro-4-(4-chlorophenoxy)phenyl]-4-methyl-1,3-dioxolan-2-yl}methyl)-1H-1,2,4-triazole, (1.039) 1-{[3-(2-chlorophenyl)-2-(2,4-difluorophenyl)oxiran-2-yl]methyl}-1H-1,2,4-triazol-5-yl thiocyanate, (1.040) 1-{[rel(2R,3R)-3-(2-chlorophenyl)-2-(2,4-difluorophenyl)oxiran-2-yl]methyl}-1H-1,2,4-triazol-5-yl thiocyanate, (1.041) 1-{[rel(2R,3S)-3-(2-chlorophenyl)-2-(2,4-difluorophenyl)oxiran-2-yl]methyl}-1H-1,2,4-triazol-5-yl thiocyanate, (1.042) 2-[(2R,4R,5R)-1-(2,4-dichlorophenyl)-5-hydroxy-2,6,6-trimethylheptan-4-yl]-2,4-dihydro-3H-1,2,4-triazole-3-thione, (1.043) 2-[(2R,4R,5S)-1-(2,4-dichlorophenyl)-5-hydroxy-2,6,6-trimethylheptan-4-yl]-2,4-dihydro-3H-1,2,4-triazole-3-thione, (1.044) 2-[(2R,4S,5R)-1-(2,4-dichlorophenyl)-5-hydroxy-2,6,6-trimethylheptan-4-yl]-2,4-dihydro-3H-1,2,4-triazole-3-thione, (1.045) 2-[(2R,4S,5S)-1-(2,4-dichlorophenyl)-5-hydroxy-2,6,6-trimethylheptan-4-yl]-2,4-dihydro-3H-1,2,4-triazole-3-thione, (1.046) 2-[(2S,4R,5R)-1-(2,4-dichlorophenyl)-5-hydroxy-2,6,6-trimethylheptan-4-yl]-2,4-dihydro-3H-1,2,4-triazole-3-thione, (1.047) 2-[(2S,4R,5S)-1-(2,4-dichlorophenyl)-5-hydroxy-2,6,6-trimethylheptan-4-yl]-2,4-dihydro-3H-1,2,4-triazole-3-thione, (1.048) 2-[(2S,4S,5R)-1-(2,4-dichlorophenyl)-5-hydroxy-2,6,6-trimethylheptan-4-yl]-2,4-dihydro-3H-1,2,4-triazole-3-thione, (1.049) 2-[(2S,4S,5S)-1-(2,4-dichlorophenyl)-5-hydroxy-2,6,6-trimethylheptan-4-yl]-2,4-dihydro-3H-1,2,4-triazole-3-thione, (1.050) 2-[1-(2,4-dichlorophenyl)-5-hydroxy-2,6,6-trimethylheptan-4-yl]-2,4-dihydro-3H-1,2,4-triazole-3-thione, (1.051) 2-[2-chloro-4-(2,4-dichlorophenoxy)phenyl]-1-(1H-1,2,4-triazol-1-yl)propan-2-ol, (1.052) 2-[2-chloro-4-(4-chlorophenoxy)phenyl]-1-(1H-1,2,4-triazol-1-yl)butan-2-ol, (1.053) 2-[4-(4-chlorophenoxy)-2-(trifluoromethyl)phenyl]-1-(1H-1,2,4-triazol-1-yl)butan-2-ol, (1.054) 2-[4-(4-chlorophenoxy)-2-(trifluoromethyl)phenyl]-1-(1H-1,2,4-triazol-1-yl)pentan-2-ol, (1.055) 2-[4-(4-chlorophenoxy)-2-(trifluoromethyl)phenyl]-1-(1H-1,2,4-triazol-1-yl)propan-2-ol, (1.056) 2-{[3-(2-chlorophenyl)-2-(2,4-difluorophenyl)oxiran-2-yl]methyl}-2,4-dihydro-3H-1,2,4-triazole-3-thione, (1.057) 2-{[rel(2R,3R)-3-(2-chlorophenyl)-2-(2,4-difluorophenyl)oxiran-2-yl]methyl}-2,4-dihydro-3H-1,2,4-triazole-3-thione, (1.058) 2-{[rel(2R,3S)-3-(2-chlorophenyl)-2-(2,4-difluorophenyl)oxiran-2-yl]methyl}-2,4-dihydro-3H-1,2,4-triazole-3-thione, (1.059) 5-(4-chlorobenzyl)-2-(chloromethyl)-2-methyl-1-(1H-1,2,4-triazol-1-ylmethyl)cyclopentanol, (1.060) 5-(allylsulfanyl)-1-{[3-(2-chlorophenyl)-2-(2,4-difluorophenyl)oxiran-2-yl]methyl}-1H-1,2,4-triazole, (1.061) 5-(allylsulfanyl)-1-{[rel(2R,3R)-3-(2-chlorophenyl)-2-(2,4-difluorophenyl)oxiran-2-yl]methyl}-1H-1,2,4-triazole, (1.062) 5-(allylsulfanyl)-1-{[rel(2R,3S)-3-(2-chlorophenyl)-2-(2,4-difluorophenyl)oxiran-2-yl]methyl}-1H-1,2,4-triazole, (1.063) N′-(2,5-dimethyl-4-{[3-(1,1,2,2-tetrafluoroethoxy)phenyl]sulfanyl}phenyl)-N-ethyl-N-methylimidoformamide, (1.064) N′-(2,5-dimethyl-4-{[3-(2,2,2-trifluoroethoxy)phenyl]sulfanyl}phenyl)-N-ethyl-N-methylimidoformamide, (1.065) N′-(2,5-dimethyl-4-{[3-(2,2,3,3-tetrafluoropropoxy)phenyl]sulfanyl}phenyl)-N-ethyl-N-methylimidoformamide, (1.066) N′-(2,5-dimethyl-4-{[3-(pentafluoroethoxy)phenyl]sulfanyl}phenyl)-N-ethyl-N-methylimidoformamide, (1.067) N′-(2,5-dimethyl-4-{3-[(1,1,2,2-tetrafluoroethyl)sulfanyl]phenoxy}phenyl)-N-ethyl-N-methylimidoformamide, (1.068) N′-(2,5-dimethyl-4-{3-[(2,2,2-trifluoroethyl)sulfanyl]phenoxy}phenyl)-N-ethyl-N-methylimidoformamide, (1.069) N′-(2,5-dimethyl-4-{3-[(2,2,3,3-tetrafluoropropyl)sulfanyl]phenoxy}phenyl)-N-ethyl-N-methylimidoformamide, (1.070) N′-(2,5-dimethyl-4-{3-[(pentafluoroethyl)sulfanyl]phenoxy}phenyl)-N-ethyl-N-methylimidoformamide, (1.071) N′-(2,5-dimethyl-4-phenoxyphenyl)-N-ethyl-N-methylimidoformamide, (1.072) N′-(4-{[3-(difluoromethoxy)phenyl]sulfanyl}-2,5-dimethylphenyl)-N-ethyl-N-methylimidoformamide, (1.073) N′-(4-{3-[(difluoromethyl)sulfanyl]phenoxy}-2,5-dimethylphenyl)-N-ethyl-N-methylimidoformamide, (1.074) N′-[5-bromo-6-(2,3-dihydro-1H-inden-2-yloxy)-2-methylpyridin-3-yl]-N-ethyl-N-methylimidoformamide, (1.075) N′-{4-[(4,5-dichloro-1,3-thiazol-2-yl) oxy]-2,5-dimethylphenyl}-N-ethyl-N-methylimidoformamide, (1.076) N′-{5-bromo -6-[(1R)-1-(3,5-difluorophenyl)ethoxy]-2-methylpyridin-3-yl}-N-ethyl-N-methylimidoformamide, (1.077) N′-{5-bromo-6-[(1S)-1-(3,5-difluorophenyl)ethoxy]-2-methylpyridin-3-yl}-N-ethyl-N-methylimidoformamide, (1.078) N′-{5-bromo-6-[(cis-4-isopropylcyclohexyl)oxy]-2-methylpyridin-3-yl}-N-ethyl-N-methylimido formamide, (1.079) N′-{5-bromo-6-[(trans-4-isopropylcyclohexyl)oxy]-2-methylpyridin-3-yl}-N-ethyl-N-methylimidoformamide, (1.080) N′-{5-bromo-6-[1-(3,5-difluorophenyl)ethoxy]-2-methylpyridin-3-yl}-N-ethyl-N-methylimidoformamide, (1.081) Mefentrifluconazole, (1.082) Ipfentrifluconazole.

2) Inhibitors of the respiratory chain at complex I or II, for example (2.001) benzovindiflupyr, (2.002) bixafen, (2.003) boscalid, (2.004) carboxin, (2.005) fluopyram, (2.006) flutolanil, (2.007) fluxapyroxad, (2.008) furametpyr, (2.009) Isofetamid, (2.010) isopyrazam (anti-epimeric enantiomer 1R,4S,9S), (2.011) isopyrazam (anti-epimeric enantiomer 1S,4R,9R), (2.012) isopyrazam (anti-epimeric racemate 1RS,4SR,9SR), (2.013) isopyrazam (mixture of syn-epimeric racemate 1RS,4SR,9RS and anti-epimeric racemate 1RS,4SR,9SR), (2.014) isopyrazam (syn-epimeric enantiomer 1R,4S,9R), (2.015) isopyrazam (syn-epimeric enantiomer 1S,4R,9S), (2.016) isopyrazam (syn-epimeric racemate 1RS,4SR,9RS), (2.017) penflufen, (2.018) penthiopyrad, (2.019) pydiflumetofen, (2.020) Pyraziflumid, (2.021) sedaxane, (2.022) 1,3-dimethyl-N-(1,1,3-trimethyl-2,3-dihydro-1H-inden-4-yl)-1H-pyrazole-4-carboxamide, (2.023) 1,3-dimethyl-N-[(3R)-1,1,3-trimethyl-2,3-dihydro-1H-inden-4-yl]-1H-pyrazole-4-carboxamide, (2.024) 1,3-dimethyl-N-[(3S)-1,1,3-trimethyl-2,3-dihydro-1H-inden-4-yl]-1H-pyrazole-4-carboxamide, (2.025) 1-methyl-3-(trifluoromethyl)-N-[2′-(trifluoromethyl)biphenyl-2-yl]-1H-pyrazole-4-carboxamide, (2.026) 2-fluoro-6-(trifluoromethyl)-N-(1,1,3-trimethyl-2,3-dihydro-1H-inden-4-yl)benzamide, (2.027) 3-(difluoromethyl)-1-methyl-N-(1,1,3-trimethyl-2,3-dihydro-1H-inden-4-yl)-1H-pyrazole-4-carboxamide, (2.028) 3-(difluoromethyl)-1-methyl-N-[(3R)-1,1,3-trimethyl-2,3-dihydro-1H-inden-4-yl]-1H-pyrazole-4-carboxamide, (2.029) 3-(difluoromethyl)-1-methyl-N-[(3S)-1,1,3-trimethyl-2,3-dihydro-1H-inden-4-yl]-1H-pyrazole-4-carboxamide, (2.030) 3-(difluoromethyl)-N-(7-fluoro-1,1,3-trimethyl-2,3-dihydro-1H-inden-4-yl)-1-methyl-1H-pyrazole-4-carboxamide, (2.031) 3-(difluoromethyl)-N-[(3R)-7-fluoro-1,1,3-trimethyl-2,3-dihydro-1H-inden-4-yl]-1-methyl-1H-pyrazole-4-carboxamide, (2.032) 3-(difluoromethyl)-N-[(3S)-7-fluoro-1,1,3-trimethyl-2,3-dihydro-1H-inden-4-yl]-1-methyl-1H-pyrazole-4-carboxamide, (2.033) 5,8-difluoro-N-[2-(2-fluoro-4-{[4-(trifluoromethyl)pyridin-2-yl]oxy}phenyl)ethyl]quinazolin-4-amine, (2.034) N-(2-cyclopentyl-5-fluorobenzyl)-N-cyclopropyl-3-(difluoromethyl)-5-fluoro-1-methyl-1H-pyrazole-4-carboxamide, (2.035) N-(2-tert-butyl-5-methylbenzyl)-N-cyclopropyl-3-(difluoromethyl)-5-fluoro-1-methyl-1H-pyrazole-4-carboxamide, (2.036) N-(2-tert-butylbenzyl)-N-cyclopropyl-3-(difluoromethyl)-5-fluoro-1-methyl-1H-pyrazole-4-carboxamide, (2.037) N-(5-chloro-2-ethylbenzyl)-N-cyclopropyl-3-(difluoromethyl)-5-fluoro-1-methyl-1H-pyrazole-4-carboxamide, (2.038) N-(5-chloro-2-isopropylbenzyl)-N-cyclopropyl-3-(difluoromethyl)-5-fluoro-1-methyl-1H-pyrazole-4-carboxamide, (2.039) N-[(1R,4S)-9-(dichloromethylene)-1,2,3,4-tetrahydro-1,4-methanonaphthalen-5-yl]-3-(difluoromethyl)-1-methyl-1H-pyrazole-4-carboxamide, (2.040) N-[(1S,4R)-9-(dichloromethylene)-1,2,3,4-tetrahydro-1,4-methanonaphthalen-5-yl]-3-(difluoromethyl)-1-methyl-1H-pyrazole-4-carboxamide, (2.041) N-[1-(2,4-dichlorophenyl)-1-methoxypropan-2-yl]-3-(difluoromethyl)-1-methyl-1H-pyrazole-4-carboxamide, (2.042) N-[2-chloro-6-(trifluoromethyl)benzyl]-N-cyclopropyl-3-(difluoromethyl)-5-fluoro-1-methyl-1H-pyrazole-4-carboxamide, (2.043) N-[3-chloro-2-fluoro-6-(trifluoromethyl)benzyl]-N-cyclopropyl-3-(difluoromethyl)-5-fluoro-1-methyl-1H-pyrazole-4-carboxamide, (2.044) N-[5-chloro-2-(trifluoromethyl)benzyl]-N-cyclopropyl-3-(difluoromethyl)-5-fluoro-1-methyl-1H-pyrazole-4-carboxamide, (2.045) N-cyclopropyl-3-(difluoromethyl)-5-fluoro-1-methyl-N-[5-methyl-2-(trifluoromethyl)benzyl]-1H-pyrazole-4-carboxamide, (2.046) N-cyclopropyl-3-(difluoromethyl)-5-fluoro-N-(2-fluoro-6-isopropylbenzyl)-1-methyl-1H-pyrazole-4-carboxamide, (2.047) N-cyclopropyl-3-(difluoromethyl)-5-fluoro-N-(2-isopropyl-5-methylbenzyl)-1-methyl-1H-pyrazole-4-carboxamide, (2.048) N-cyclopropyl-3-(difluoromethyl)-5-fluoro-N-(2-isopropylbenzyl)-1-methyl-1H-pyrazole-4-carbothioamide, (2.049) N-cyclopropyl-3-(difluoromethyl)-5-fluoro-N-(2-isopropylbenzyl)-1-methyl-1H-pyrazole-4-carboxamide, (2.050) N-cyclopropyl-3-(difluoromethyl)-5-fluoro-N-(5-fluoro-2-isopropylbenzyl)-1-methyl-1H-pyrazole-4-carboxamide, (2.051) N-cyclopropyl-3-(difluoromethyl)-N-(2-ethyl-4,5-dimethylbenzyl)-5-fluoro-1-methyl-1H-pyrazole-4-carboxamide, (2.052) N-cyclopropyl-3-(difluoromethyl)-N-(2-ethyl-5-fluorobenzyl)-5-fluoro-1-methyl-1H-pyrazole-4-carboxamide, (2.053) N-cyclopropyl-3-(difluoromethyl)-N-(2-ethyl-5-methylbenzyl)-5-fluoro-1-methyl-1H-pyrazole-4-carboxamide, (2.054) N-cyclopropyl-N-(2-cyclopropyl-5-fluorobenzyl)-3-(difluoromethyl)-5-fluoro-1-methyl-1H-pyrazole-4-carboxamide, (2.055) N-cyclopropyl-N-(2-cyclopropyl-5-methylbenzyl)-3-(difluoromethyl)-5-fluoro-1-methyl-1H-pyrazole-4-carboxamide, (2.056) N-cyclopropyl-N-(2-cyclopropylbenzyl)-3-(difluoromethyl)-5-fluoro-1-methyl-1H-pyrazole-4-carboxamide.

3) Inhibitors of the respiratory chain at complex III, for example (3.001) ametoctradin, (3.002) amisulbrom, (3.003) azoxystrobin, (3.004) coumethoxystrobin, (3.005) coumoxystrobin, (3.006) cyazofamid, (3.007) dimoxystrobin, (3.008) enoxastrobin, (3.009) famoxadone, (3.010) fenamidone, (3.011) flufenoxystrobin, (3.012) fluoxastrobin, (3.013) kresoxim-methyl, (3.014) metominostrobin, (3.015) orysastrobin, (3.016) picoxystrobin, (3.017) pyraclostrobin, (3.018) pyrametostrobin, (3.019) pyraoxystrobin, (3.020) trifloxystrobin, (3.021) (2E)-2-{2-[({[(1E)-1-(3-{[(E)-1-fluoro-2-phenylvinyl]oxy}phenyl)ethylidene]amino}oxy)methyl]phenyl}-2-(methoxyimino)-N-methylacetamide, (3.022) (2E,3Z)-5-{[1-(4-chlorophenyl)-1H-pyrazol-3-yl]oxy}-2-(methoxyimino)-N,3-dimethylpent-3-enamide, (3.023) (2R)-2-{2-[(2,5-dimethylphenoxy)methyl]phenyl}-2-methoxy-N-methylacetamide, (3.024) (2S)-2-{2-[(2,5-dimethylphenoxy)methyl]phenyl}-2-methoxy-N-methylacetamide, (3.025) (3S,6S,7R,8R)-8-benzyl-3-[({3-[(isobutyryloxy)methoxy]-4-methoxypyridin-2-yl}carbonyl)amino]-6-methyl-4,9-dioxo-1,5-dioxonan-7-yl 2-methylpropanoate, (3.026) 2-{2-[(2,5-dimethylphenoxy)methyl]phenyl}-2-methoxy-N-methylacetamide, (3.027) N-(3-ethyl-3,5,5-trimethylcyclohexyl)-3-formamido-2-hydroxybenzamide, (3.028) (2E,3Z)-5-{[1-(4-chloro-2-fluorophenyl)-1H-pyrazol-3-yl]oxy}-2-(methoxyimino)-N,3-dimethylpent-3-enamide, (3.029) methyl {5-[3-(2,4-dimethylphenyl)-1H-pyrazol-1-yl]-2-methylbenzyl}carbamate.

4) Inhibitors of the mitosis and cell division, for example (4.001) carbendazim, (4.002) diethofencarb, (4.003) ethaboxam, (4.004) fluopicolide, (4.005) pencycuron, (4.006) thiabendazole, (4.007) thiophanate-methyl, (4.008) zoxamide, (4.009) 3-chloro-4-(2,6-difluorophenyl)-6-methyl-5-phenylpyridazine, (4.010) 3-chloro-5-(4-chlorophenyl)-4-(2,6-difluorophenyl)-6-methylpyridazine, (4.011) 3-chloro-5-(6-chloropyridin-3-yl)-6-methyl-4-(2,4,6-trifluorophenyl)pyridazine, (4.012) 4-(2-bromo-4-fluorophenyl)-N-(2,6-difluorophenyl)-1,3-dimethyl-1H-pyrazol-5-amine, (4.013) 4-(2-bromo-4-fluorophenyl)-N-(2-bromo-6-fluorophenyl)-1,3-dimethyl-1H-pyrazol-5-amine, (4.014) 4-(2-bromo-4-fluorophenyl)-N-(2-bromophenyl)-1,3-dimethyl-1H-pyrazol-5-amine, (4.015) 4-(2-bromo-4-fluorophenyl)-N-(2-chloro-6-fluorophenyl)-1,3-dimethyl-1H-pyrazol-5-amine, (4.016) 4-(2-bromo-4-fluorophenyl)-N-(2-chlorophenyl)-1,3-dimethyl-1H-pyrazol-5-amine, (4.017) 4-(2-bromo-4-fluorophenyl)-N-(2-fluorophenyl)-1,3-dimethyl-1H-pyrazol-5-amine, (4.018) 4-(2-chloro-4-fluorophenyl)-N-(2,6-difluorophenyl)-1,3-dimethyl-1H-pyrazol-5-amine, (4.019) 4-(2-chloro-4-fluorophenyl)-N-(2-chloro-6-fluorophenyl)-1,3-dimethyl-1H-pyrazol-5-amine, (4.020) 4-(2-chloro-4-fluorophenyl)-N-(2-chlorophenyl)-1,3-dimethyl-1H-pyrazol-5-amine, (4.021) 4-(2-chloro-4-fluorophenyl)-N-(2-fluorophenyl)-1,3-dimethyl-1H-pyrazol-5-amine, (4.022) 4-(4-chlorophenyl)-5-(2,6-difluorophenyl)-3,6-dimethylpyridazine, (4.023) N-(2-bromo-6-fluorophenyl)-4-(2-chloro-4-fluorophenyl)-1,3-dimethyl-1H-pyrazol-5-amine, (4.024) N-(2-bromophenyl)-4-(2-chloro-4-fluorophenyl)-1,3-dimethyl-1H-pyrazol-5-amine, (4.025) N-(4-chloro-2,6-difluorophenyl)-4-(2-chloro-4-fluorophenyl)-1,3-dimethyl-1H-pyrazol-5-amine.

5) Compounds capable to have a multisite action, for example (5.001) bordeaux mixture, (5.002) captafol, (5.003) captan, (5.004) chlorothalonil, (5.005) copper hydroxide, (5.006) copper naphthenate, (5.007) copper oxide, (5.008) copper oxychloride, (5.009) copper(2+) sulfate, (5.010) dithianon, (5.011) dodine, (5.012) folpet, (5.013) mancozeb, (5.014) maneb, (5.015) metiram, (5.016) metiram zinc, (5.017) oxine-copper, (5.018) propineb, (5.019) sulfur and sulfur preparations including calcium polysulfide, (5.020) thiram, (5.021) zineb, (5.022) ziram, (5.023) 6-ethyl-5,7-dioxo-6,7-dihydro-5H-pyrrolo[3′,4′:5,6][1,4]dithiino[2,3-c][1,2]thiazole-3-carbonitrile.

6) Compounds capable to induce a host defence, for example (6.001) acibenzolar-S-methyl, (6.002) isotianil, (6.003) probenazole, (6.004) tiadinil.

7) Inhibitors of the amino acid and/or protein biosynthesis, for example (7.001) cyprodinil, (7.002) kasugamycin, (7.003) kasugamycin hydrochloride hydrate, (7.004) oxytetracycline, (7.005) pyrimethanil, (7.006) 3-(5-fluoro-3,3,4,4-tetramethyl-3,4-dihydroisoquinolin-1-yl)quinoline.

8) Inhibitors of the ATP production, for example (8.001) silthiofam.

9) Inhibitors of the cell wall synthesis, for example (9.001) benthiavalicarb, (9.002) dimethomorph, (9.003) flumorph, (9.004) iprovalicarb, (9.005) mandipropamid, (9.006) pyrimorph, (9.007) valifenalate, (9.008) (2E)-3-(4-tert-butylphenyl)-3-(2-chloropyridin-4-yl)-1-(morpholin-4-yl)prop-2-en-1-one, (9.009) (2Z)-3-(4-tert-butylphenyl)-3-(2-chloropyridin-4-yl)-1-(morpholin-4-yl)prop-2-en-1-one.

10) Inhibitors of the lipid and membrane synthesis, for example (10.001) propamocarb, (10.002) propamocarb hydrochloride, (10.003) tolclofos-methyl.

11) Inhibitors of the melanin biosynthesis, for example (11.001) tricyclazole, (11.002) 2,2,2-trifluoroethyl {3-methyl-1-[(4-methylbenzoyl)amino]butan-2-yl}carbamate.

12) Inhibitors of the nucleic acid synthesis, for example (12.001) benalaxyl, (12.002) benalaxyl-M (kiralaxyl), (12.003) metalaxyl, (12.004) metalaxyl-M (mefenoxam).

13) Inhibitors of the signal transduction, for example (13.001) fludioxonil, (13.002) iprodione, (13.003) procymidone, (13.004) proquinazid, (13.005) quinoxyfen, (13.006) vinclozolin.

14) Compounds capable to act as an uncoupler, for example (14.001) fluazinam, (14.002) meptyldinocap.

15) Further compounds, for example (15.001) Abscisic acid, (15.002) benthiazole, (15.003) bethoxazin, (15.004) capsimycin, (15.005) carvone, (15.006) chinomethionat, (15.007) cufraneb, (15.008) cyflufenamid, (15.009) cymoxanil, (15.010) cyprosulfamide, (15.011) flutianil, (15.012) fosetyl-aluminium, (15.013) fosetyl-calcium, (15.014) fosetyl-sodium, (15.015) methyl isothiocyanate, (15.016) metrafenone, (15.017) mildiomycin, (15.018) natamycin, (15.019) nickel dimethyldithiocarbamate, (15.020) nitrothal-isopropyl, (15.021) oxamocarb, (15.022) Oxathiapiprolin, (15.023) oxyfenthiin, (15.024) pentachlorophenol and salts, (15.025) phosphorous acid and its salts, (15.026) propamocarbfosetylate, (15.027) pyriofenone (chlazafenone), (15.028) tebufloquin, (15.029) tecloftalam, (15.030) tolnifanide, (15.031) 1-(4-{4-[(5R)-5-(2,6-difluorophenyl)-4,5-dihydro-1,2-oxazol-3-yl]-1,3-thiazol-2-yl}piperidin-1-yl)-2-[5-methyl-3-(trifluoromethyl)-1H-pyrazol-1-yl]ethanone, (15.032) 1-(4-{4-[(5S)-5-(2,6-difluorophenyl)-4,5-dihydro-1,2-oxazol-3-yl]-1,3-thiazol-2-yl}piperidin-1-yl)-2-[5-methyl-3-(trifluoromethyl)-1H-pyrazol-1-yl]ethanone, (15.033) 2-(6-benzylpyridin-2-yl)quinazoline, (15.034) 2,6-dimethyl-1H,5H-[1,4]dithiino[2,3-c:5,6-c′]dipyrrole-1,3,5,7(2H,6H)-tetrone, (15.035) 2-[3,5-bis(difluoromethyl)-1H-pyrazol-1-yl]-1-[4-(4-{5-[2-(prop-2-yn-1-yloxy)phenyl]-4,5-dihydro-1,2-oxazol-3-yl}-1,3-thiazol-2-yl)piperidin-1-yl]ethanone, (15.036) 2-[3,5-bis(difluoromethyl)-1H-pyrazol-1-yl]-1-[4-(4-{5-[2-chloro-6-(prop-2-yn-1-yloxy)phenyl]-4,5-dihydro-1,2-oxazol-3-yl}-1,3-thiazol-2-yl)piperidin-1-yl]ethanone, (15.037) 2-[3,5-bis(difluoromethyl)-1H-pyrazol-1-yl]-1-[4-(4-{5-[2-fluoro-6-(prop-2-yn-1-yloxy)phenyl]-4,5-dihydro-1,2-oxazol-3-yl}-1,3-thiazol-2-yl)piperidin-1-yl]ethanone, (15.038) 2-[6-(3-fluoro-4-methoxyphenyl)-5-methylpyridin-2-yl]quinazoline, (15.039) 2-{(5R)-3-[2-(1-{[3,5-bis(difluoromethyl)-1H-pyrazol-1-yl]acetyl}piperidin-4-yl)-1,3-thiazol-4-yl]-4,5-dihydro-1,2-oxazol-5-yl}-3-chlorophenyl methanesulfonate, (15.040) 2-{(5S)-3-[2-(1-{[3,5-bis(difluoromethyl)-1H-pyrazol-1-yl]acetyl}piperidin-4-yl)-1,3-thiazol-4-yl]-4,5-dihydro-1,2-oxazol-5-yl}-3-chlorophenyl methanesulfonate, (15.041) 2-{2-[(7,8-difluoro-2-methylquinolin-3-yl)oxy]-6-fluorophenyl}propan-2-ol, (15.042) 2-{2-fluoro-6-[(8-fluoro-2-methylquinolin-3-yl)oxy]phenyl}propan-2-ol, (15.043) 2-{3-[2-(1-{[3,5-bis(difluoromethyl)-1H-pyrazol-1-yl]acetyl}piperidin-4-yl)-1,3-thiazol-4-yl]-4,5-dihydro-1,2-oxazol-5-yl}-3-chlorophenyl methanesulfonate, (15.044) 2-{3-[2-(1-{[3,5-bis(difluoromethyl)-1H-pyrazol-1-yl]acetyl}piperidin-4-yl)-1,3-thiazol-4-yl]-4,5-dihydro-1,2-oxazol-5-yl}phenyl methanesulfonate, (15.045) 2-phenylphenol and salts, (15.046) 3-(4,4,5-trifluoro-3,3-dimethyl-3,4-dihydroisoquinolin-1-yl)quinoline, (15.047) 3-(4,4-difluoro-3,3-dimethyl-3,4-dihydroisoquinolin-1-yl)quinoline, (15.048) 4-amino-5-fluoropyrimidin-2-ol (tautomeric form: 4-amino-5-fluoropyrimidin-2(1H)-one), (15.049) 4-oxo-4-[(2-phenylethyl)amino]butanoic acid, (15.050) 5-amino-1,3,4-thiadiazole-2-thiol, (15.051) 5-chloro-N′-phenyl-N′-(prop-2-yn-1-yl)thiophene-2-sulfonohydrazide, (15.052) 5-fluoro-2-[(4-fluorobenzyl)oxy]pyrimidin-4-amine, (15.053) 5-fluoro-2-[(4-methylbenzyl)oxy]pyrimidin-4-amine, (15.054) 9-fluoro-2,2-dimethyl-5-(quinolin-3-yl)-2,3-dihydro-1,4-benzoxazepine, (15.055) but-3-yn-1-yl {6-[({[(Z)-(1-methyl-1H-tetrazol-5-yl)(phenyl)methylene]amino}oxy)methyl]pyridin-2-yl}carbamate, (15.056) ethyl (2Z)-3-amino-2-cyano-3-phenylacrylate, (15.057) phenazine-1-carboxylic acid, (15.058) propyl 3,4,5-trihydroxybenzoate, (15.059) quinolin-8-ol, (15.060) quinolin-8-ol sulfate (2:1), (15.061) tert-butyl {6-[({[(1-methyl-1H-tetrazol-5-yl)(phenyl)methylene]amino}oxy)methyl]pyridin-2-yl}carbamate, (15.062) 5-fluoro-4-imino-3-methyl-1-[(4-methylphenyl)sulfonyl]-3,4-dihydropyrimidin-2(1H)-one.

All named mixing partners of the classes (1) to (15) as described here above can be present in the form of the free compound and/or, if their functional groups enable this, an agriculturally acceptable salt thereof.

Where a compound (A) or a compound (B) can be present in tautomeric form, such a compound is understood hereinabove and hereinbelow also to include, where applicable, corresponding tautomeric forms, even when these are not specifically mentioned in each case.

The active ingredients specified herein by their Common Name are known and described, for example, in The Pesticide Manual (16th Ed. British Crop Protection Council) or can be searched in the internet (e.g. www.alanwood.net/pesticides).

### Methods and Uses

The compound and the composition of the invention have potent microbicidal activity. They can be used for controlling unwanted microorganisms, such as unwanted phytopathogenic fungi and bacteria. They can be particularly useful in crop protection (they control microorganisms that cause plants diseases) or for protecting materials (e.g. industrial materials, timber, storage goods) as described in more details herein below. More specifically, the compound and the composition of the invention can be used to protect seeds, germinating plants, emerged seedlings, plants, plant parts, fruits and the soil in which the plants grow from unwanted microorganisms, in particular from phytopathogenic fungi.

Control or controlling as used herein encompasses curative and protective treatment of unwanted microorganisms. Unwanted microorganisms may be pathogenic bacteria or pathogenic fungi, more specifically phytopathogenic bacteria or phytopathogenic fungi. As detailed herein below, these phytopathogenic microorganisms are the causal agents of a broad spectrum of plants diseases.

More specifically, the compound and the composition of the invention can be used as fungicides. In particular, they can be useful in crop protection, for example for the control of unwanted fungi, such as Plasmodiophoromycetes, Oomycetes, Chytridiomycetes, Zygomycetes, Ascomycetes, Basidiomycetes and Deuteromycetes.

The compound and the composition of the invention can also be used as bactericide. In particular, they can be used in crop protection, for example for the control of unwanted bacteria, such as Pseudomonadaceae, Rhizobiaceae, Enterobacteriaceae, Corynebacteriaceae and Streptomycetaceae.

The present invention also relates to a method for controlling unwanted microorganisms, such as unwanted fungi and bacteria, in particular phytopathogenic fungi, comprising the step of applying at least one compound of the invention or at least one composition of the invention to the microorganisms and/or their habitat (to the plants, plant parts, seeds, fruits or to the soil in which the plants grow).

Typically, when the compound and the composition of the invention are used in curative or protective methods for controlling phytopathogenic fungi, an effective and non-phytotoxic amount thereof is applied to the plants, plant parts, fruits, seeds or to the soil in which the plants grow.

Effective and non-phytotoxic amount means an amount that is sufficient to control or destroy the fungi present or liable to appear on the cropland and that does not entail any appreciable symptom of phytotoxicity for said crops. Such an amount can vary within a wide range depending on the fungus to be controlled, the type of crop, the climatic conditions and the respective compound or composition of the invention used. This amount can be determined by systematic field trials that are within the capabilities of a person skilled in the art.

### Plants and Plant Parts

The compound and the composition of the invention can be applied to any plants or plant parts.

Plants mean all plants and plant populations, such as desired and undesired wild plants or crop plants (including naturally occurring crop plants). Crop plants may be plants which can be obtained by conventional breeding and optimization methods or by biotechnological and genetic engineering methods or combinations of these methods, including the genetically modified plants (GMO or transgenic plants) and the plant cultivars which are protectable and non-protectable by plant breeders' rights.

### Genetically Modified Plants (GMO)

Genetically modified plants (GMO or transgenic plants) are plants of which a heterologous gene has been stably integrated into the genome. The expression “heterologous gene” essentially means a gene which is provided or assembled outside the plant and when introduced in the nuclear, chloroplastic or mitochondrial genome. This gene gives the transformed plant new or improved agronomic or other properties by expressing a protein or polypeptide of interest or by downregulating or silencing other gene(s) which are present in the plant (using for example, antisense technology, cosuppression technology, RNA interference—RNAi—technology or microRNA—miRNA—technology). A heterologous gene that is located in the genome is also called a transgene. A transgene that is defined by its particular location in the plant genome is called a transformation or transgenic event.

Plant cultivars are understood to mean plants which have new properties (“traits”) and have been obtained by conventional breeding, by mutagenesis or by recombinant DNA techniques. They can be cultivars, varieties, bio- or genotypes.

Plant parts are understood to mean all parts and organs of plants above and below the ground, such as shoots, leaves, needles, stalks, stems, flowers, fruit bodies, fruits, seeds, roots, tubers and rhizomes. The plant parts also include harvested material and vegetative and generative propagation material, for example cuttings, tubers, rhizomes, slips and seeds.

Plants which can be treated in accordance with the methods of the invention include the following: cotton, flax, grapevine, fruit, vegetables, such as Rosaceae sp. (for example pome fruits such as apples and pears, but also stone fruits such as apricots, cherries, almonds and peaches, and soft fruits such as strawberries), Ribesioidae sp., Juglandaceae sp., Betulaceae sp., Anacardiaceae sp., Fagaceae sp., Moraceae sp., Oleaceae sp., Actinidaceae sp., Lauraceae sp., Musaceae sp. (for example banana trees and plantations), Rubiaceae sp. (for example coffee), Theaceae sp., Sterculiceae sp., Rutaceae sp. (for example lemons, oranges and grapefruit); Solanaceae sp. (for example tomatoes), Liliaceae sp., Asteraceae sp. (for example lettuce), Umbelliferae sp., Cruciferae sp., Chenopodiaceae sp., Cucurbitaceae sp. (for example cucumber), Alliaceae sp. (for example leek, onion), Papilionaceae sp. (for example peas); major crop plants, such as Gramineae sp. (for example maize, turf, cereals such as wheat, rye, rice, barley, oats, millet and triticale), Asteraceae sp. (for example sunflower), Brassicaceae sp. (for example white cabbage, red cabbage, broccoli, cauliflower, Brussels sprouts, pak Choi, kohlrabi, radishes, and oilseed rape, mustard, horseradish and cress), Fabacae sp. (for example bean, peanuts), Papilionaceae sp. (for example soya bean), Solanaceae sp. (for example potatoes), Chenopodiaceae sp. (for example sugar beet, fodder beet, swiss chard, beetroot); useful plants and ornamental plants for gardens and wooded areas; and genetically modified varieties of each of these plants.

### Pathogens

Non-limiting examples of pathogens of fungal diseases which can be treated in accordance with the invention include:

diseases caused by powdery mildew pathogens, for example Blumeria species, for example Blumeria graminis; Podosphaera species, for example Podosphaera leucotricha; Sphaerotheca species, for example Sphaerotheca fuliginea; Uncinula species, for example Uncinula necator;

diseases caused by rust disease pathogens, for example Gymnosporangium species, for example Gymnosporangium sabinae; Hemileia species, for example Hemileia vastatrix; Phakopsora species, for example Phakopsora pachyrhizi or Phakopsora meibomiae; Puccinia species, for example Puccinia recondita, Puccinia graminis oder Puccinia striiformis; Uromyces species, for example Uromyces appendiculatus;

diseases caused by pathogens from the group of the Oomycetes, for example Albugo species, for example Albugo candida; Bremia species, for example Bremia lactucae; Peronospora species, for example Peronospora pisi or P. brassicae; Phytophthora species, for example Phytophthora infestans; Plasmopara species, for example Plasmopara viticola; Pseudoperonospora species, for example Pseudoperonospora humuli or Pseudoperonospora cubensis; Pythium species, for example Pythium ultimum;

leaf blotch diseases and leaf wilt diseases caused, for example, by Alternaria species, for example Alternaria solani; Cercospora species, for example Cercospora beticola; Cladiosporium species, for example Cladiosporium cucumerinum; Cochliobolus species, for example Cochliobolus sativus (conidial form: Drechslera, syn: Helminthosporium) or Cochliobolus miyabeanus; Colletotrichum species, for example Colletotrichum lindemuthanium; Cycloconium species, for example Cycloconium oleaginum; Diaporthe species, for example Diaporthe citri; Elsinoe species, for example Elsinoe fawcettii; Gloeosporium species, for example Gloeosporium laeticolor; Glomerella species, for example Glomerella cingulata; Guignardia species, for example Guignardia bidwelli; Leptosphaeria species, for example Leptosphaeria maculans; Magnaporthe species, for example Magnaporthe grisea; Microdochium species, for example Microdochium nivale; Mycosphaerella species, for example Mycosphaerella graminicola, Mycosphaerella arachidicola or Mycosphaerella fijiensis; Phaeosphaeria species, for example Phaeosphaeria nodorum; Pyrenophora species, for example Pyrenophora teres or Pyrenophora tritici repentis; Ramularia species, for example Ramularia collo-cygni or Ramularia areola; Rhynchosporium species, for example Rhynchosporium secalis; Septoria species, for example Septoria apii or Septoria lycopersici; Stagonospora species, for example Stagonospora nodorum; Typhula species, for example Typhula incarnata; Venturia species, for example Venturia inaequalis;

root and stem diseases caused, for example, by Corticium species, for example Corticium graminearum; Fusarium species, for example Fusarium oxysporum; Gaeumannomyces species, for example Gaeumannomyces graminis; Plasmodiophora species, for example Plasmodiophora brassicae; Rhizoctonia species, for example Rhizoctonia solani; Sarocladium species, for example Sarocladium oryzae; Sclerotium species, for example Sclerotium oryzae; Tapesia species, for example Tapesia acuformis; Thielaviopsis species, for example Thielaviopsis basicola;

ear and panicle diseases (including corn cobs) caused, for example, by Alternaria species, for example Alternaria spp.; Aspergillus species, for example Aspergillus flavus; Cladosporium species, for example Cladosporium cladosporioides; Claviceps species, for example Claviceps purpurea; Fusarium species, for example Fusarium culmorum; Gibberella species, for example Gibberella zeae; Monographella species, for example Monographella nivalis; Stagnospora species, for example Stagnospora nodorum;

diseases caused by smut fungi, for example Sphacelotheca species, for example Sphacelotheca reiliana; Tilletia species, for example Tilletia caries or Tilletia controversa; Urocystis species, for example Urocystis occulta; Ustilago species, for example Ustilago nuda;

fruit rot caused, for example, by Aspergillus species, for example Aspergillus flavus; Botrytis species, for example Botrytis cinerea; Penicillium species, for example Penicillium expansum or Penicillium purpurogenum; Rhizopus species, for example Rhizopus stolonifer; Sclerotinia species, for example Sclerotinia sclerotiorum; Verticilium species, for example Verticilium alboatrum;

seed- and soil-borne rot and wilt diseases, and also diseases of seedlings, caused, for example, by Alternaria species, for example Alternaria brassicicola; Aphanomyces species, for example Aphanomyces euteiches; Ascochyta species, for example Ascochyta lentis; Aspergillus species, for example Aspergillus flavus; Cladosporium species, for example Cladosporium herbarum; Cochliobolus species, for example Cochliobolus sativus (conidial form: Drechslera, Bipolaris Syn: Helminthosporium); Colletotrichum species, for example Colletotrichum coccodes; Fusarium species, for example Fusarium culmorum; Gibberella species, for example Gibberella zeae; Macrophomina species, for example Macrophomina phaseolina; Microdochium species, for example Microdochium nivale; Monographella species, for example Monographella nivalis; Penicillium species, for example Penicillium expansum; Phoma species, for example Phoma lingam; Phomopsis species, for example Phomopsis sojae; Phytophthora species, for example Phytophthora cactorum; Pyrenophora species, for example Pyrenophora graminea; Pyricularia species, for example Pyricularia oryzae; Pythium species, for example Pythium ultimum; Rhizoctonia species, for example Rhizoctonia solani; Rhizopus species, for example Rhizopus oryzae; Sclerotium species, for example Sclerotium rolfsii; Septoria species, for example Septoria nodorum; Typhula species, for example Typhula incarnata; Verticillium species, for example Verticillium dahliae;

cancers, galls and witches' broom caused, for example, by Nectria species, for example Nectria galligena;

wilt diseases caused, for example, by Monilinia species, for example Monilinia laxa;

deformations of leaves, flowers and fruits caused, for example, by Exobasidium species, for example Exobasidium vexans; Taphrina species, for example Taphrina deformans;

degenerative diseases in woody plants, caused, for example, by Esca species, for example Phaeomoniella chlamydospora, Phaeoacremonium aleophilum or Fomitiporia mediterranea; Ganoderma species, for example Ganoderma boninense;

diseases of flowers and seeds caused, for example, by Botrytis species, for example Botrytis cinerea;

diseases of plant tubers caused, for example, by Rhizoctonia species, for example Rhizoctonia solani; Helminthosporium species, for example Helminthosporium solani;

diseases caused by bacterial pathogens, for example Xanthomonas species, for example Xanthomonas campestris pv. oryzae; Pseudomonas species, for example Pseudomonas syringae pv. lachrymans; Erwinia species, for example Erwinia amylovora.

diseases of soya beans:

Fungal diseases on leaves, stems, pods and seeds caused, for example, by Alternaria leaf spot (Alternaria spec. atrans tenuissima), Anthracnose (Colletotrichum gloeosporoides dematium var. truncatum), brown spot (Septoria glycines), cercospora leaf spot and blight (Cercospora kikuchii), choanephora leaf blight (Choanephora infundibulifera trispora (Syn.)), dactuliophora leaf spot (Dactuliophora glycines), downy mildew (Peronospora manshurica), drechslera blight (Drechslera glycini), frogeye leaf spot (Cercospora sojina), leptosphaerulina leaf spot (Leptosphaerulina trifolii), phyllostica leaf spot (Phyllosticta sojaecola), pod and stem blight (Phomopsis sojae), powdery mildew (Microsphaera diffusa), pyrenochaeta leaf spot (Pyrenochaeta glycines), rhizoctonia aerial, foliage, and web blight (Rhizoctonia solani), rust (Phakopsora pachyrhizi, Phakopsora meibomiae), scab (Sphaceloma glycines), stemphylium leaf blight (Stemphylium botryosum), target spot (Corynespora cassiicola).

Fungal diseases on roots and the stem base caused, for example, by black root rot (Calonectria crotalariae), charcoal rot (Macrophomina phaseolina), fusarium blight or wilt, root rot, and pod and collar rot (Fusarium oxysporum, Fusarium orthoceras, Fusarium semitectum, Fusarium equiseti), mycoleptodiscus root rot (Mycoleptodiscus terrestris), neocosmospora (Neocosmospora vasinfecta), pod and stem blight (Diaporthe phaseolorum), stem canker (Diaporthe phaseolorum var. caulivora), phytophthora rot (Phytophthora megasperma), brown stem rot (Phialophora gregata), pythium rot (Pythium aphanidermatum, Pythium irregulare, Pythium debaryanum, Pythium myriotylum, Pythium ultimum), rhizoctonia root rot, stem decay, and damping-off (Rhizoctonia solani), sclerotinia stem decay (Sclerotinia sclerotiorum), sclerotinia southern blight (Sclerotinia rolfsii), thielaviopsis root rot (Thielaviopsis basicola).

### Mycotoxins

In addition, the compound and the composition of the invention can reduce the mycotoxin content in the harvested material and the foods and feeds prepared therefrom. Mycotoxins include particularly, but not exclusively, the following: deoxynivalenol (DON), nivalenol, 15-Ac-DON, 3-Ac-DON, T2- and HT2-toxin, fumonisins, zearalenon, moniliformin, fusarin, diaceotoxyscirpenol (DAS), beauvericin, enniatin, fusaroproliferin, fusarenol, ochratoxins, patulin, ergot alkaloids and aflatoxins which can be produced, for example, by the following fungi: Fusarium spec., such as F. acuminatum, F. asiaticum, F. avenaceum, F. crookwellense, F. culmorum, F. graminearum (Gibberella zeae), F. equiseti, F. fujikoroi, F. musarum, F. oxysporum, F. proliferatum, F. poae, F. pseudograminearum, F. sambucinum, F. scirpi, F. semitectum, F. solani, F. sporotrichoides, F. langsethiae, F. subglutinans, F. tricinctum, F. verticillioides etc., and also by Aspergillus spec., such as A. flavus, A. parasiticus, A. nomius, A. ochraceus, A. clavatus, A. terreus, A. versicolor, Penicillium spec., such as P. verrucosum, P. viridicatum, P. citrinum, P. expansum, P. claviforme, P. roqueforti, Claviceps spec., such as C. purpurea, C. fusiformis, C. paspali, C. africana, Stachybotrys spec. and others.

### Material Protection

The compound and the composition of the invention can also be used in the protection of materials, especially for the protection of industrial materials against attack and destruction by phytopathogenic fungi.

In addition, the compound and the composition of the invention can be used as antifouling compositions, alone or in combinations with other active ingredients.

Industrial materials in the present context are understood to mean inanimate materials which have been prepared for use in industry. For example, industrial materials which are to be protected from microbial alteration or destruction may be adhesives, glues, paper, wallpaper and board/cardboard, textiles, carpets, leather, wood, fibers and tissues, paints and plastic articles, cooling lubricants and other materials which can be infected with or destroyed by microorganisms. Parts of production plants and buildings, for example cooling-water circuits, cooling and heating systems and ventilation and air-conditioning units, which may be impaired by the proliferation of microorganisms may also be mentioned within the scope of the materials to be protected. Industrial materials within the scope of the present invention preferably include adhesives, sizes, paper and card, leather, wood, paints, cooling lubricants and heat transfer fluids, more preferably wood.

The compound and the composition of the invention may prevent adverse effects, such as rotting, decay, discoloration, decoloration or formation of mould.

In the case of treatment of wood the compound and the composition of the invention may also be used against fungal diseases liable to grow on or inside timber.

Timber means all types of species of wood, and all types of working of this wood intended for construction, for example solid wood, high-density wood, laminated wood, and plywood. In addition, the compound and the composition of the invention can be used to protect objects which come into contact with saltwater or brackish water, especially hulls, screens, nets, buildings, moorings and signalling systems, from fouling.

The compound and the composition of the invention can also be employed for protecting storage goods. Storage goods are understood to mean natural substances of vegetable or animal origin or processed products thereof which are of natural origin, and for which long-term protection is desired. Storage goods of vegetable origin, for example plants or plant parts, such as stems, leaves, tubers, seeds, fruits, grains, can be protected freshly harvested or after processing by (pre)drying, moistening, comminuting, grinding, pressing or roasting. Storage goods also include timber, both unprocessed, such as construction timber, electricity poles and barriers, or in the form of finished products, such as furniture. Storage goods of animal origin are, for example, hides, leather, furs and hairs. The compound and the composition of the invention may prevent adverse effects, such as rotting, decay, discoloration, decoloration or formation of mould.

Microorganisms capable of degrading or altering industrial materials include, for example, bacteria, fungi, yeasts, algae and slime organisms. The compound and the composition of the invention preferably act against fungi, especially moulds, wood-discoloring and wood-destroying fungi (Ascomycetes, Basidiomycetes, Deuteromycetes and Zygomycetes), and against slime organisms and algae. Examples include microorganisms of the following genera: Alternaria, such as Alternaria tenuis; Aspergillus, such as Aspergillus niger; Chaetomium, such as Chaetomium globosum; Coniophora, such as Coniophora puetana; Lentinus, such as Lentinus tigrinus; Penicillium, such as Penicillium glaucum; Polyporus, such as Polyporus versicolor; Aureobasidium, such as Aureobasidium pullulans; Sclerophoma, such as Sclerophoma pityophila; Trichoderma, such as Trichoderma viride; Ophiostoma spp., Ceratocystis spp., Humicola spp., Petriella spp., Trichurus spp., Coriolus spp., Gloeophyllum spp., Pleurotus spp., Poria spp., Serpula spp. and Tyromyces spp., Cladosporium spp., Paecilomyces spp. Mucor spp., Escherichia, such as Escherichia coli; Pseudomonas, such as Pseudomonas aeruginosa; Staphylococcus, such as Staphylococcus aureus, Candida spp. and Saccharomyces spp., such as Saccharomyces cerevisae.

### Seed Treatment

The compound and the composition of the invention may also be used to protect seeds from unwanted microorganisms, such as phytopathogenic microorganisms, for instance phytopathogenic fungi. The term seed(s) as used herein include dormant seeds, primed seeds, pregerminated seeds and seeds with emerged roots and leaves.

Thus, the present invention also relates to a method for protecting seeds from unwanted microorganisms, in particular from unwanted phytopathogenic fungi which comprises the step of treating the seeds with the compound or the composition of the invention.

The treatment of seeds with the compound or the composition of the invention protects the seeds from phytopathogenic microorganisms, but also protects the germinating plants, the emerged seedlings and the plants after emergence from the treated seeds. Therefore, the present invention also relates to a method for protecting seeds, germinating plants and emerged seedlings.

The seeds treatment may be performed prior to sowing, at the time of sowing or shortly thereafter.

When the seeds treatment is performed prior to sowing (e.g. so-called on-seed applications), the seeds treatment may be performed as follows: the seeds may be placed into a mixer with a desired amount of the compound or the composition of the invention, the seeds and the compound or the composition of the invention are mixed until an homogeneous distribution on seeds is achieved. If appropriate, the seeds may then be dried.

The invention also relates to seeds treated with the compound or the composition of the invention.

Preferably, the seeds are treated in a state in which it is sufficiently stable for no damage to occur in the course of treatment. In general, seeds can be treated at any time between harvest and shortly after sowing. It is customary to use seeds which have been separated from the plant and freed from cobs, shells, stalks, coats, hairs or the flesh of the fruits. For example, it is possible to use seeds which have been harvested, cleaned and dried down to a moisture content of less than 15% by weight. Alternatively, it is also possible to use seeds which, after drying, for example, have been treated with water and then dried again, or seeds just after priming, or seeds stored in primed conditions or pre-germinated seeds, or seeds sown on nursery trays, tapes or paper.

The amount of the compound or the composition of the invention applied to the seeds is typically such that the germination of the seed is not impaired, or that the resulting plant is not damaged. This must be ensured particularly in case the the compound of the invention would exhibit phytotoxic effects at certain application rates. The intrinsic phenotypes of transgenic plants should also be taken into consideration when determining the amount of the compound of the invention to be applied to the seed in order to achieve optimum seed and germinating plant protection with a minimum amount of compound being employed.

The compound of the invention can be applied as such, directly to the seeds, i.e. without the use of any other components and without having been diluted. Also the composition of the invention can be applied to the seeds.

The compound and the composition of the invention are suitable for protecting seeds of any plant variety. Preferred seeds are that of cereals (such as wheat, barley, rye, millet, triticale, and oats), oilseed rape, maize, cotton, soybean, rice, potatoes, sunflower, beans, coffee, peas, beet (e.g. sugar beet and fodder beet), peanut, vegetables (such as tomato, cucumber, onions and lettuce), lawns and ornamental plants. More preferred are seeds of wheat, soybean, oilseed rape, maize and rice.

The compound and the composition of the invention can be used for treating transgenic seeds, in particular seeds of plants capable of expressing a polypeptide or protein which acts against pests, herbicidal damage or abiotic stress, thereby increasing the protective effect. Seeds of plants capable of expressing a polypeptide or protein which acts against pests, herbicidal damage or abiotic stress may contain at least one heterologous gene which allows the expression of said polypeptide or protein. These heterologous genes in transgenic seeds may originate, for example, from microorganisms of the species Bacillus, Rhizobium, Pseudomonas, Serratia, Trichoderma, Clavibacter, Glomus or Gliocladium. These heterologous genes preferably originate from Bacillus sp., in which case the gene product is effective against the European corn borer and/or the Western corn rootworm. Particularly preferably, the heterologous genes originate from Bacillus thuringiensis.

### Application

The compound of the invention can be applied as such, or for example in the form of as ready-to-use solutions, emulsions, water- or oil-based suspensions, powders, wettable powders, pastes, soluble powders, dusts, soluble granules, granules for broadcasting, suspoemulsion concentrates, natural products impregnated with the compound of the invention, synthetic substances impregnated with the compound of the invention, fertilizers or microencapsulations in polymeric substances.

Application is accomplished in a customary manner, for example by watering, spraying, atomizing, broadcasting, dusting, foaming, spreading-on and the like. It is also possible to deploy the compound of the invention by the ultra-low volume method or to inject it into the soil.

The effective and non-phytotoxic amount of the compound of the invention which is applied to the plants, plant parts, fruits, seeds or soil will depend on various factors, such as the compound/composition employed, the subject of the treatment (plant, plant part, fruit, seed or soil), the type of treatment (dusting, spraying, seed dressing), the purpose of the treatment (curative and protective) and the type of microorganisms.

When the compound of the invention is used as a fungicide, the application rates can vary within a relatively wide range, depending on the kind of application. For the treatment of plant parts, such as leaves, the application rate may range from 0.1 to 10 000 g/ha, preferably from 10 to 1000 g/ha, more preferably from 50 to 300 g/ha (in the case of application by watering or dripping, it is even possible to reduce the application rate, especially when inert substrates such as rockwool or perlite are used). For the treatment of seeds, the application rate may range from 0.1 to 200 g per 100 kg of seeds, preferably from Ito 150 g per 100 kg of seeds, more preferably from 2.5 to 25 g per 100 kg of seeds, even more preferably from 2.5 to 12.5 g per 100 kg of seeds. For the treatment of soil, the application rate may range from 0.1 to 10 000 g/ha, preferably from 1 to 5000 g/ha.

These application rates are merely examples and are not intended to limit the scope of the present invention.

The compounds of formula (I) can thus be used to protect plants from attack by the pathogens mentioned for a certain period of time after treatment. The period for which protection is provided extends generally for 1 to 28 days, preferably for 1 to 14 days, more preferably for 1 to 10 days, most preferably for 1 to 7 days, after the treatment of the plants with the compounds of formula (I), or for up to 200 days after a seed treatment.

The plants listed herein can particularly be treated in accordance with the invention with the compounds of formula (I). The preferred ranges stated above for the a compounds of formula (I) also apply to the treatment of these plants. Particular emphasis is given to the treatment of plants with the active compound combinations or compositions specifically mentioned in the present text.

### Antimycotic Effects

The compound and the composition of the invention also have very good antimycotic effects. They have a very broad antimycotic activity spectrum, especially against dermatophytes and yeasts, moulds and diphasic fungi (for example against Candida species, such as Candida albicans, Candida glabrata), and Epidermophyton floccosum, Aspergillus species, such as Aspergillus niger and Aspergillus fumigatus, Trichophyton species, such as Trichophyton mentagrophytes, Microsporon species such as Microsporon canis and audouinii. The enumeration of these fungi by no means constitutes a restriction of the mycotic spectrum covered, and is merely of illustrative character.

The compound and the composition of the invention can also be used to control important fungal pathogens in fish and crustacea farming, e.g. saprolegnia diclina in trouts, saprolegnia parasitica in crayfish.

The compound and the composition of the invention can therefore be used both in medical and in non-medical applications.

### Plant Growth Regulation

The compound and the composition of the invention can, at particular concentrations or application rates, also be used as herbicides, safeners, growth regulators or agents to improve plant properties, or as microbicides, for example as bactericides, viricides (including compositions against viroids) or as compositions against MLO (Mycoplasma-like organisms) and RLO (Rickettsia-like organisms).

The compound and the composition of the invention may intervene in physiological processes of plants and can therefore also be used as plant growth regulators. Plant growth regulators may exert various effects on plants. The effect of the substances depends essentially on the time of application in relation to the developmental stage of the plant, and also on the amounts of active ingredient applied to the plants or their environment and on the type of application. In each case, growth regulators should have a particular desired effect on the crop plants.

Growth regulating effects, comprise earlier germination, better emergence, more developed root system and/or improved root growth, increased ability of tillering, more productive tillers, earlier flowering, increased plant height and/or biomass, shorting of stems, improvements in shoot growth, number of kernels/ear, number of ears/m², number of stolons and/or number of flowers, enhanced harvest index, bigger leaves, less dead basal leaves, improved phyllotaxy, earlier maturation/earlier fruit finish, homogenous riping, increased duration of grain filling, better fruit finish, bigger fruit/vegetable size, sprouting resistance and reduced lodging.

Increased or improved yield is referring to total biomass per hectare, yield per hectare, kernel/fruit weight, seed size and/or hectolitre weight as well as to improved product quality, comprising:

improved processability relating to size distribution (kernel, fruit, etc.), homogenous riping, grain moisture, better milling, better vinification, better brewing, increased juice yield, harvestability, digestibility, sedimentation value, falling number, pod stability, storage stability, improved fiber length/strength/uniformity, increase of milk and/or meet quality of silage fed animals, adaption to cooking and frying;

improved marketability relating to improved fruit/grain quality, size distribution (kernel, fruit, etc.), increased storage/shelf-life, firmness/softness, taste (aroma, texture, etc.), grade (size, shape, number of berries, etc.), number of berries/fruits per bunch, crispness, freshness, coverage with wax, frequency of physiological disorders, colour, etc.;

increased desired ingredients such as e.g. protein content, fatty acids, oil content, oil quality, aminoacid composition, sugar content, acid content (pH), sugar/acid ratio (Brix), polyphenols, starch content, nutritional quality, gluten content/index, energy content, taste, etc.;

decreased undesired ingredients such as e.g. less mycotoxines, less aflatoxines, geosmin level, phenolic aromas, lacchase, polyphenol oxidases and peroxidases, nitrate content etc.

Plant growth-regulating compounds can be used, for example, to slow down the vegetative growth of the plants. Such growth depression is of economic interest, for example, in the case of grasses, since it is thus possible to reduce the frequency of grass cutting in ornamental gardens, parks and sport facilities, on roadsides, at airports or in fruit crops. Also of significance is the inhibition of the growth of herbaceous and woody plants on roadsides and in the vicinity of pipelines or overhead cables, or quite generally in areas where vigorous plant growth is unwanted.

Also important is the use of growth regulators for inhibition of the longitudinal growth of cereal. This reduces or completely eliminates the risk of lodging of the plants prior to harvest. In addition, growth regulators in the case of cereals can strengthen the culm, which also counteracts lodging. The employment of growth regulators for shortening and strengthening culms allows the deployment of higher fertilizer volumes to increase the yield, without any risk of lodging of the cereal crop.

In many crop plants, vegetative growth depression allows denser planting, and it is thus possible to achieve higher yields based on the soil surface. Another advantage of the smaller plants obtained in this way is that the crop is easier to cultivate and harvest.

Reduction of the vegetative plant growth may also lead to increased or improved yields because the nutrients and assimilates are of more benefit to flower and fruit formation than to the vegetative parts of the plants.

Alternatively, growth regulators can also be used to promote vegetative growth. This is of great benefit when harvesting the vegetative plant parts. However, promoting vegetative growth may also promote generative growth in that more assimilates are formed, resulting in more or larger fruits.

Furthermore, beneficial effects on growth or yield can be achieved through improved nutrient use efficiency, especially nitrogen (N)-use efficiency, phosphours (P)-use efficiency, water use efficiency, improved transpiration, respiration and/or CO₂ assimilation rate, better nodulation, improved Ca-metabolism etc.

Likewise, growth regulators can be used to alter the composition of the plants, which in turn may result in an improvement in quality of the harvested products. Under the influence of growth regulators, parthenocarpic fruits may be formed. In addition, it is possible to influence the sex of the flowers. It is also possible to produce sterile pollen, which is of great importance in the breeding and production of hybrid seed.

Use of growth regulators can control the branching of the plants. On the one hand, by breaking apical dominance, it is possible to promote the development of side shoots, which may be highly desirable particularly in the cultivation of ornamental plants, also in combination with an inhibition of growth. On the other hand, however, it is also possible to inhibit the growth of the side shoots. This effect is of particular interest, for example, in the cultivation of tobacco or in the cultivation of tomatoes.

Under the influence of growth regulators, the amount of leaves on the plants can be controlled such that defoliation of the plants is achieved at a desired time. Such defoliation plays a major role in the mechanical harvesting of cotton, but is also of interest for facilitating harvesting in other crops, for example in viticulture. Defoliation of the plants can also be undertaken to lower the transpiration of the plants before they are transplanted.

Furthermore, growth regulators can modulate plant senescence, which may result in prolonged green leaf area duration, a longer grain filling phase, improved yield quality, etc.

Growth regulators can likewise be used to regulate fruit dehiscence. On the one hand, it is possible to prevent premature fruit dehiscence. On the other hand, it is also possible to promote fruit dehiscence or even flower abortion to achieve a desired mass (“thinning”). In addition it is possible to use growth regulators at the time of harvest to reduce the forces required to detach the fruits, in order to allow mechanical harvesting or to facilitate manual harvesting.

Growth regulators can also be used to achieve faster or else delayed ripening of the harvested material before or after harvest. This is particularly advantageous as it allows optimal adjustment to the requirements of the market. Moreover, growth regulators in some cases can improve the fruit colour. In addition, growth regulators can also be used to synchronize maturation within a certain period of time. This establishes the prerequisites for complete mechanical or manual harvesting in a single operation, for example in the case of tobacco, tomatoes or coffee.

By using growth regulators, it is additionally possible to influence the resting of seed or buds of the plants, such that plants such as pineapple or ornamental plants in nurseries, for example, germinate, sprout or flower at a time when they are normally not inclined to do so. In areas where there is a risk of frost, it may be desirable to delay budding or germination of seeds with the aid of growth regulators, in order to avoid damage resulting from late frosts.

Finally, growth regulators can induce resistance of the plants to frost, drought or high salinity of the soil. This allows the cultivation of plants in regions which are normally unsuitable for this purpose.

### Resistance Induction/Plant Health and Other effects

The compound and the composition of the invention also exhibit a potent strengthening effect in plants. Accordingly, they can be used for mobilizing the defences of the plant against attack by undesirable microorganisms.

Plant-strengthening (resistance-inducing) substances in the present context are substances capable of stimulating the defence system of plants in such a way that the treated plants, when subsequently inoculated with undesirable microorganisms, develop a high degree of resistance to these microorganisms.

Further, in context with the present invention plant physiology effects comprise the following:

Abiotic stress tolerance, comprising tolerance to high or low temperatures, drought tolerance and recovery after drought stress, water use efficiency (correlating to reduced water consumption), flood tolerance, ozone stress and UV tolerance, tolerance towards chemicals like heavy metals, salts, pesticides etc.

Biotic stress tolerance, comprising increased fungal resistance and increased resistance against nematodes, viruses and bacteria. In context with the present invention, biotic stress tolerance preferably comprises increased fungal resistance and increased resistance against nematodes.

Increased plant vigor, comprising plant health/plant quality and seed vigor, reduced stand failure, improved appearance, increased recovery after periods of stress, improved pigmentation (e.g. chlorophyll content, stay-green effects, etc.) and improved photosynthetic efficiency.

### PREPARATION EXAMPLES

The preparation and the use of the inventive active ingredients of the formula (I) is illustrated by the examples which follow. However, the invention is not limited to these examples.

### General Procedure for Step (a)

### 2-Chloro-5-methyl-4-(4,4,5,5-tetramethyl-1,3,2-dioxaborolan-2-yl)aniline

A mixture of 4-bromo-2-chloro-5-methylaniline (1.5 g, 6.8 mmol, 1 eq.), bis(pinacol)diboron (2.59 g, 10.2 mmol, 1.5 eq.), Pd(dppf)Cl₂ (0.75 g, 1.02 mmol, 0.15 eq.) and potassium acetate (2 g, 20.4 mmol, 3 eq.) in DMF (30 mL) was stirred under argon at 95° C. for 16 hours. After completion of the reaction, the mixture was diluted with water and extracted with ethyl acetate. The combined organic layer was washed with brine solution, dried over anhydrous sodium sulfate and the solvent was removed under reduced pressure. Purification by column chromatography (ethyl acetate/c-hexane) afforded the title compound (1.92 g, 84% yield).

### General Procedure for Step (b)

### 2-Chloro-4-[(2-fluorophenyl)methyl]-5-methyl-aniline

A mixture of 2-fluorobenzylchloride (435 mg, 3 mmol, 1 eq.), 2-chloro-5-methyl-4-(4,4,5,5-tetramethyl-1,3,2-dioxaborolan-2-yl)aniline (966 mg, 3.61 mmol, 1.2 eq.), cesium carbonate (3.92 g, 12 mmol, 4 eq.) and tetrakis(triphenylposphine)palladium (70 mg, 0.06 mmol, 0.02 eq.) in 1-butanol (20 ml) and water (5 mL) was stirred under argon at 80° C. for 16 hours. After completion, the mixture was filtered, diluted with water and extracted with ethyl acetate. The combined organic phases were washed with brine solution, dried over anhydrous sodium sulfate and the solvent was removed under reduced pressure. Purification by column chromatography (ethyl acetate/c-hexane) afforded the title compound (373 mg, 41% yield).

### General Procedure for Step (c)

### 1,4-Dimethyl-2-nitro-5-(1-phenylvinyl)benzene

A mixture of 1-phenylvinylboronic acid (1 g, 6.75 mmol, 1.25 eq.), 4-bromo-2,5-dimethylnitrobenzene (1.24 g, 5.4 mmol, 1 eq.) and tetrakis(triphenylposphine)palladium (0.32 g, 0.28 mmol, 0.05 eq.) in toluene (10 mL) and 2M sodium carbonate (8 mL) was stirred under argon at 90° C. for 4 hours. After completion, the mixture was filtered, diluted with water and extracted with ethyl acetate. The combined organic phases were washed with brine solution, dried over anhydrous sodium sulfate and the solvent was removed under reduced pressure. Purification by column chromatography (ethyl acetate/c-hexane) afforded the title compound (917 mg, 49% yield).

### General Procedure for Step (d)—Cyclopropyl

### 1,4-Dimethyl-2-nitro-5-(1-phenylcyclopropyl)benzene

A 1.5M diethylzinc solution (2.63 mL, 4 mmol, 2 eq.) was added at 0° C. to a solution of 1,4-dimethyl-2-nitro-5-(1-phenylvinyl)benzene (500 mg, 2 mmol, 1 eq.) in DCM (5 mL) followed by diiodomethane (0.32 mL, 4 mmol, 2 eq.) and the resulting mixture was stirred at room temperature for 16 hours. After completion, the mixture was diluted with water, neutralized with 1M HCl and extracted with ethyl acetate. The combined organic phases were washed with brine solution, dried over anhydrous sodium sulfate and the solvent was removed under reduced pressure to afford the crude title compound (640 mg) which was used directly in the next step.

### General Procedure for Step (d)—Difluorocyclopropyl

### 1-(2,2-Difluoro-1-phenyl-cyclopropyl)-2,5-dimethyl-4-nitro-benzene

A mixture of 1,4-dimethyl-2-nitro-5-(1-phenylvinyl)benzene (1.3 g, 5.1 mmol, 1 eq.) and sodium bromodifluoroacetate (2.02 g, 10.3 mmol, 2 eq.) in diethylenglycoldimethylether (50 mL) was stirred at 150° C. for 13 hours. The mixture was diluted with water and extracted with ethyl acetate. The combined organic phases were washed with brine solution, dried over anhydrous sodium sulfate and the solvent was removed under reduced pressure. Purification by column chromatography (ethyl acetate/c-hexane) afforded the title compound (240 mg, 14% yield).

### General Procedure for Step (d)—Dibromocyclopropyl

### 1-(2,2-Dibromo-1-phenyl-cyclopropyl)-2,5-dimethyl-4-nitro-benzene

Bromoform (0.69 mL, 7.9 mmol, 8 eq.) was added to a mixture of 1,4-dimethyl-2-nitro-5-(1-phenylvinyl)benzene (250 mg, 0.98 mmol, 1 eq.) and tetra(n-butyl)ammonium bromide (16 mg, 0.04 mmol, 0.05 eq.) in conc. sodium hydroxide (0.6 mL) and the resulting mixture was stirred at 50° C. for 16 hours. The mixture was diluted with water and extracted with ethyl acetate. The combined organic phases were washed with brine solution, dried over anhydrous sodium sulfate and the solvent was removed under reduced pressure. Purification by column chromatography (ethyl acetate/c-hexane) afforded the title compound (470 mg, 94% yield).

### General Procedure for Step (e)

### 4-(2,2-Difluoro-1-phenyl-cyclopropyl)-2,5-dimethyl-aniline

A mixture of 1-(2,2-difluoro-1-phenyl-cyclopropyl)-2,5-dimethyl-4-nitro-benzene (240 mg, 0.79 mmol, 1 eq.) and tin chloride dihydrate (893 mg, 3.96 mmol, 5 eq.) in ethanol (15 mL) was stirred at reflux for 30 min. After completion, the mixture was diluted with water, neutralized with sodium carbonate and extracted with ethyl acetate. The combined organic phases were washed with brine solution, dried over anhydrous sodium sulfate and the solvent was removed under reduced pressure to afford the crude title compound (140 mg, 56% yield) which was used directly in the next step.

### General Procedure for Step (f)

### N′-[4-[(2-Cyanophenyl)methyl]-2,5-dimethyl-phenyl]-N-ethyl-N-methyl-formamidine (Ex No 8)

A mixture of 2-[(4-amino-2,5-dimethyl-phenyl)methyl]benzonitrile (180 mg, 0.76 mmol, 1 eq.) and N-(dimethoxymethyl)-N-methyl-ethanamine (152 mg, 1.14 mmol, 1.5 eq.) in touene (5 mL) was stirred at 80° C. for 4 hours. After completion, the mixture was diluted with water and extracted with ethyl acetate. The combined organic phases were washed with brine solution, dried over anhydrous sodium sulfate and the solvent was removed under reduced pressure. Purification by column chromatography (ethyl acetate/c-hexane) afforded the title compound (164 mg, 70% yield).

### General Procedure for Step (g)

### 2-chloro-4-[(2-chlorophenyl)methyl]-5-methyl-aniline

Under Argon, a 0.5M solution of 2-chlorobenzylzinc chloride (4 mL, 2 mmol, 1.1 eq) was added to a suspension of 4-bromo-2-chloro-5-methyl-aniline (400 mg, 1.81 mmol, 1 eq), Pd(OAc)₂ (4 mg, 0.02 mmol, 0.01 eq) and S-Phos (15 mg, 0.04 mmol, 0.02 eq) in THF (5 mL) and the resulting reaction mixture was stirred at room temperature for 24 hours. After completion, the reaction was diluted with ethyl acetate, washed successively with water and brine, dried over sodium sulfate and concentrated in vacuo. Purification by preparative HPLC afforded the title compound (252 mg, 52% yield).

### EXAMPLES

(I)

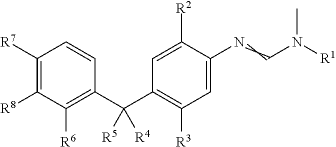

| Ex No | LogP | NMR PeakList | IUPAC name |
|---|---|---|---|
| 1 | 1.41[a]; | Example 1: 1H-NMR(400.0 MHz, d6-DMSO): | N′-[2-chloro-4-[(2- |
| | 3.86[b] | δ = 7.852 (2.1); 7.833 (2.2); 7.730 (1.1); 7.662 (1.0); | cyanophenyl)methyl]-5- |
| | | 7.659 (1.0); 7.642 (2.2); 7.639 (2.2); 7.623 (1.6); | methylphenyl]-N-ethyl-N- |
| | | 7.620 (1.6); 7.456 (1.4); 7.437 (2.3); 7.419 (1.0); | methylmethanimidamide |
| | | 7.211 (2.2); 7.192 (2.0); 6.944 (5.9); 6.842 (1.5); | |
| | | 5.756 (1.2); 4.079 (8.4); 3.422 (0.5); 3.347 (1.1); | |
| | | 3.320 (11.9); 2.986 (1.4); 2.918 (3.7); 2.671 (0.3); | |
| | | 2.506 (46.0); 2.502 (60.8); 2.497 (44.2); 2.328 (0.3); | |
| | | 2.146 (16.0); 1.398 (0.4); 1.149 (1.9); 1.132 (3.8); | |
| | | 1.115 (2.1); 0.008 (0.6); 0.000 (17.3) | |
| 2 | 1.52[a]; | Example 2: 1H-NMR(400.0 MHz, d6-DMSO): | N′-[2-chloro-4-[(2- |
| | 4.50[b] | δ = 7.706 (1.2); 7.592 (0.4); 7.306 (0.4); 7.300 (0.4); | fluorophenyl)methyl]-5- |
| | | 7.286 (1.0); 7.268 (1.4); 7.253 (0.7); 7.249 (0.7); | methylphenyl]-N-ethyl-N- |
| | | 7.200 (1.5); 7.177 (1.7); 7.154 (1.0); 7.148 (1.1); | methylmethanimidamide |
| | | 7.129 (2.4); 7.109 (1.9); 7.100 (1.4); 7.085 (1.6); | |
| | | 7.082 (1.5); 7.066 (0.6); 6.977 (5.3); 6.804 (1.6); | |
| | | 3.873 (7.7); 3.428 (0.5); 3.414 (0.5); 3.321 (52.1); | |
| | | 2.977 (1.4); 2.912 (3.7); 2.670 (0.4); 2.506 (55.8); | |
| | | 2.501 (72.3); 2.497 (55.0); 2.328 (0.4); 2.152 (16.0); | |
| | | 2.067 (0.4); 1.144 (2.2); 1.127 (4.4); 1.110 (2.4); | |
| | | 0.000 (64.6) | |
| 3 | 1.49[a] | Example 3: 1H-NMR(400.0 MHz, d6-DMSO): | N′-(4-benzyl-2-chloro-5- |
| | | δ = 7.696 (1.0); 7.583 (0.4); 7.301 (1.7); 7.283 (4.5); | methylphenyl)-N-ethyl-N- |
| | | 7.264 (3.3); 7.197 (1.4); 7.179 (2.2); 7.160 (0.8); | methylmethanimidamide |
| | | 7.137 (4.1); 7.118 (3.4); 7.062 (5.7); 6.778 (1.5); | |
| | | 3.865 (8.2); 3.414 (0.5); 3.321 (52.3); 2.973 (1.2); | |
| | | 2.911 (3.2); 2.670 (0.4); 2.505 (45.1); 2.501 (61.4); | |
| | | 2.497 (47.2); 2.328 (0.4); 2.123 (16.0); 2.040 (0.4); | |
| | | 1.142 (2.5); 1.125 (5.1); 1.107 (2.5); 0.008 (2.2); | |
| | | 0.000 (58.0); −0.008 (3.1) | |
| 4 | 1.38[a]; | Example 4: 1H-NMR(400.0 MHz, d6-DMSO): | N′-[5-chloro-4-[(2- |
| | 4.38[b] | δ = 7.837 (2.7); 7.819 (2.9); 7.752 (1.2); 7.638 (1.6); | cyanophenyl)methyl]-2- |
| | | 7.618 (2.8); 7.599 (1.7); 7.438 (1.8); 7.419 (3.0); | methylphenyl]-N-ethyl-N- |
| | | 7.400 (1.4); 7.186 (3.0); 7.166 (2.8); 6.999 (6.1); | methylmethanimidamide |
| | | 6.914 (1.6); 5.757 (1.3); 4.157 (11.3); 3.424 (0.6); | |
| | | 3.349 (1.3); 3.324 (36.5); 2.984 (1.5); 2.916 (3.8); | |
| | | 2.507 (38.9); 2.502 (50.6); 2.498 (36.5); 2.120 (16.0); | |
| | | 1.142 (2.6); 1.125 (5.1); 1.108 (2.7); 0.000 (8.5) | |
| 5 | 1.58[a]; | Example 5: 1H-NMR(400.0 MHz, d6-DMSO): | N′-[5-chloro-4-+(2- |
| | 2.99[b] | δ = 7.729 (1.0); 7.622 (0.4); 7.290 (0.4); 7.283 (0.6); | fluorophenyl)methyl]-2- |
| | | 7.276 (0.8); 7.270 (1.1); 7.255 (1.6); 7.248 (1.0); | methylphenyl]-N-ethyl-N- |
| | | 7.239 (0.8); 7.234 (0.8); 7.185 (1.9); 7.162 (2.0); | methylmethanimidamide |
| | | 7.159 (2.0); 7.139 (1.3); 7.131 (0.9); 7.111 (2.9); | |
| | | 7.104 (2.0); 7.096 (3.9); 7.085 (1.8); 7.081 (1.7); | |
| | | 7.067 (0.6); 6.966 (5.5); 6.879 (1.6); 3.955 (9.7); | |
| | | 3.416 (0.6); 3.331 (84.7); 2.976 (1.3); 2.910 (3.4); | |
| | | 2.671 (0.4); 2.667 (0.3); 2.506 (55.7); 2.502 (74.2); | |
| | | 2.498 (55.8); 2.329 (0.4); 2.103 (16.0); 1.232 (1.4); | |
| | | 1.138 (2.6); 1.121 (4.9); 1.104 (2.7); 0.008 (2.7); | |
| | | 0.000 (71.3); −0.008 (3.3) | |
| 6 | 1.72[a]; | Example 6: 1H-NMR(601.6 MHz, d6-DMSO): | N′-(4-benzyl-5-chloro-2- |
| | 5.16[b] | δ = 19.971 (0.3); 7.712 (0.7); 7.603 (0.3); 7.280 (3.3); | methylphenyl)-N-ethyl-N- |
| | | 7.268 (6.0); 7.260 (1.1); 7.255 (5.7); 7.179 (9.0); | methylmethanimidamide |
| | | 7.168 (7.3); 7.156 (1.3); 7.040 (6.6); 6.850 (1.1); | |
| | | 3.941 (12.8); 3.419 (0.5); 3.332 (0.9); 3.304 (70.5); | |
| | | 2.972 (0.9); 2.906 (2.3); 2.612 (0.6); 2.521 (1.1); | |
| | | 2.518 (1.4); 2.515 (1.3); 2.506 (33.9); 2.503 (77.0); | |
| | | 2.500 (108.8); 2.497 (78.1); 2.494 (35.4); 2.384 (0.6); | |
| | | 2.178 (0.5); 2.110 (16.0); 1.130 (2.1); 1.119 (4.1); | |
| | | 1.108 (2.4); 0.005 (0.4); 0.000 (17.8); −0.006 (0.5) | |
| 7 | 1.92[a]; | Example 7: 1H-NMR(400.0 MHz, d6-DMSO): | N′-[4-[(2- |
| | 5.16[b] | δ = 7.605 (0.5); 7.586 (0.4); 7.470 (0.4); 7.460 (2.1); | chlorophenyl)methyl]-2,5- |
| | | 7.452 (2.1); 7.447 (1.7); 7.437 (2.6); 7.428 (0.4); | dimethylphenyl]-N-ethyl- |
| | | 7.256 (0.7); 7.246 (5.2); 7.237 (4.0); 7.231 (3.8); | N-methylmethanimidamide |
| | | 7.223 (5.1); 7.213 (0.6); 6.958 (1.9); 6.948 (1.6); | |
| | | 6.944 (1.8); 6.935 (1.6); 6.699 (5.3); 6.602 (3.7); | |
| | | 3.907 (9.8); 3.317 (43.1); 2.918 (3.6); 2.675 (0.4); | |
| | | 2.670 (0.6); 2.666 (0.4); 2.523 (1.8); 2.510 (33.9); | |
| | | 2.506 (68.4); 2.501 (93.2); 2.497 (70.1); 2.492 (34.6); | |
| | | 2.332 (0.4); 2.328 (0.5); 2.324 (0.4); 2.087 (16.0); | |
| | | 2.072 (15.7); 1.998 (0.3); 1.988 (0.9); 1.398 (2.9); | |
| | | 1.175 (0.5); 1.136 (4.9); 1.118 (10.5); 1.100 (4.8); | |
| | | 0.008 (1.2); 0.000 (30.4); −0.008 (1.2) | |
| 8 | 1.47[a]; | Example 8: 1H-NMR(400.0 MHz, d6-DMSO): | N′-[4-[(2- |
| | 4.15[b] | δ = 7.827 (2.4); 7.825 (2.5); 7.808 (2.6); 7.806 (2.7); | cyanophenyl)methyl]-2,5- |
| | | 7.625 (1.6); 7.622 (1.6); 7.606 (3.1); 7.603 (3.1); | dimethylphenyl]-N-ethyl- |
| | | 7.586 (2.0); 7.584 (1.9); 7.421 (1.6); 7.402 (2.7); | N-methylmethanimidamide |
| | | 7.383 (1.2); 7.153 (2.7); 7.134 (2.5); 6.749 (5.4); | |
| | | 6.604 (3.8); 4.042 (9.7); 3.319 (60.2); 2.917 (4.1); | |
| | | 2.670 (0.4); 2.666 (0.4); 2.505 (55.1); 2.501 (73.1); | |
| | | 2.497 (58.4); 2.328 (0.4); 2.114 (16.0); 2.080 (15.6); | |
| | | 1.398 (1.1); 1.133 (4.7); 1.116 (9.7); 1.098 (4.5); | |
| | | 0.000 (15.6) | |
| 9 | 1.79[a]; | Example 9: 1H-NMR(400.0 MHz, d6-DMSO): | N′-[4-[(2,4- |
| | 4.86[b] | δ = 7.585 (0.5); 7.214 (1.0); 7.208 (1.0); 7.188 (1.5); | difluorophenyl)methyl]-2,5- |
| | | 7.184 (1.5); 7.164 (1.0); 7.158 (1.0); 7.074 (0.5); | dimethylphenyl]-N-ethyl- |
| | | 7.053 (1.5); 7.035 (1.7); 7.014 (1.1); 7.006 (1.3); | N-methylmethanimidamide |
| | | 7.000 (1.2); 6.984 (1.8); 6.978 (1.7); 6.963 (0.6); | |
| | | 6.957 (0.6); 6.774 (4.9); 6.574 (3.6); 3.805 (7.5); | |
| | | 3.319 (38.3); 2.912 (3.9); 2.670 (0.3); 2.505 (44.3); | |
| | | 2.501 (59.0); 2.497 (45.0); 2.328 (0.3); 2.202 (0.6); | |
| | | 2.109 (16.0); 2.080 (15.1); 1.988 (0.4); 1.398 (1.1); | |
| | | 1.130 (4.7); 1.112 (9.6); 1.094 (4.5); 0.000 (0.6) | |
| 10 | 1.60[a]; | Example 10: 1H-NMR(400.0 MHz, d6-DMSO): | N-ethyl-N′-[4-[(2- |
| | 4.87[b] | δ = 7.584 (0.5); 7.268 (0.4); 7.264 (0.4); 7.249 (1.1); | fluorophenyl)methyl]-2,5- |
| | | 7.230 (1.6); 7.225 (1.0); 7.216 (0.8); 7.211 (0.8); | dimethylphenyl]-N- |
| | | 7.172 (1.7); 7.150 (1.8); 7.147 (1.8); 7.129 (1.1); | methylmethanimidamide |
| | | 7.126 (1.1); 7.109 (1.2); 7.107 (1.2); 7.091 (2.6); | |
| | | 7.088 (2.5); 7.072 (1.6); 7.070 (1.6); 7.039 (1.2); | |
| | | 7.035 (1.2); 7.020 (1.8); 7.016 (1.8); 7.001 (0.8); | |
| | | 6.997 (0.7); 6.926 (0.4); 6.787 (4.9); 6.572 (3.7); | |
| | | 6.555 (0.4); 5.753 (0.8); 3.836 (8.1); 3.751 (0.4); | |
| | | 3.317 (31.6); 2.912 (4.2); 2.523 (0.7); 2.510 (17.2); | |
| | | 2.506 (36.0); 2.501 (50.4); 2.496 (38.2); 2.492 (18.9); | |
| | | 2.202 (1.9); 2.118 (16.0); 2.079 (15.5); 2.033 (0.9); | |
| | | 1.988 (1.0); 1.960 (1.0); 1.398 (2.9); 1.175 (0.5); | |
| | | 1.130 (5.0); 1.113 (10.7); 1.095 (4.9); 0.008 (0.9); | |
| | | 0.000 (27.5); −0.008 (1.0) | |
| 11 | 1.58[a]; | Example 11: 1H-NMR(400.0 MHz, d6-DMSO): | N′-(4-benzyl-2,5- |
| | 4.90[b] | δ = 7.570 (0.4); 7.271 (1.6); 7.253 (4.4); 7.234 (3.4); | dimethylphenyl)-N-ethyl- |
| | | 7.217 (0.4); 7.164 (1.3); 7.146 (2.1); 7.128 (1.0); | N-methylmethanimidamide |
| | | 7.116 (3.6); 7.098 (3.1); 7.075 (0.3); 6.855 (4.2); | |
| | | 6.694 (0.4); 6.546 (3.1); 6.401 (0.5); 4.558 (0.5); | |
| | | 3.833 (7.2); 3.749 (0.8); 3.318 (22.6); 2.910 (3.6); | |
| | | 2.523 (0.6); 2.509 (11.1); 2.505 (23.3); 2.500 (32.6); | |
| | | 2.496 (24.5); 2.491 (11.9); 2.202 (0.7); 2.095 (16.0); | |
| | | 2.011 (1.5); 1.979 (1.5); 1.128 (4.4); 1.111 (9.6); | |
| | | 1.093 (4.3); 0.008 (0.6); 0.000 (19.2); −0.008 (0.7) | |
| 12 | 1.92[a]; | Example 12: 1H-NMR(400.0 MHz, d6-DMSO): | N′-[4-[(2,4- |
| | 5.31[b] | δ = 7.650 (0.5); 7.214 (1.0); 7.207 (1.0); 7.188 (1.4); | difluorophenyl)methyl]-2,5- |
| | | 7.183 (1.5); 7.164 (1.0); 7.158 (1.0); 7.074 (0.6); | dimethylphenyl]-N- |
| | | 7.053 (1.6); 7.036 (1.6); 7.014 (1.1); 7.005 (1.3); | isopropyl-N- |
| | | 6.999 (1.3); 6.984 (1.8); 6.978 (1.7); 6.963 (0.7); | methylmethanimidamide |
| | | 6.956 (0.6); 6.774 (4.9); 6.575 (3.5); 3.804 (7.4); | |
| | | 3.720 (0.4); 3.318 (40.6); 2.818 (14.0); 2.670 (0.4); | |
| | | 2.524 (0.9); 2.510 (23.9); 2.506 (50.3); 2.501 (70.3); | |
| | | 2.497 (53.3); 2.492 (26.4); 2.332 (0.3); 2.328 (0.4); | |
| | | 2.324 (0.3); 2.203 (0.6); 2.157 (0.4); 2.110 (16.0); | |
| | | 2.079 (15.1); 2.023 (0.8); 1.961 (0.8); 1.398 (0.3); | |
| | | 1.172 (8.3); 1.156 (8.3); 0.000 (0.8) | |
| 13 | 1.85[a] | Example 13: 1H-NMR(400.0 MHz, d6-DMSO): | N′-[2,5-dimethyl-4-(1- |
| | | δ = 7.601 (0.5); 7.204 (2.2); 7.186 (4.9); 7.166 (3.7); | phenylcyclopropyl)phenyl]- |
| | | 7.090 (1.9); 7.075 (6.5); 7.053 (1.1); 6.936 (4.8); | N-ethyl-N- |
| | | 6.918 (4.2); 6.903 (0.4); 6.554 (3.8); 3.322 (35.8); | methylmethanimidamide |
| | | 2.922 (4.4); 2.506 (43.1); 2.501 (57.6); 2.497 (43.1); | |
| | | 2.328 (0.3); 2.149 (15.5); 2.064 (16.0); 2.024 (0.6); | |
| | | 1.989 (0.6); 1.398 (4.1); 1.292 (1.3); 1.275 (4.6); | |
| | | 1.266 (2.0); 1.225 (0.6); 1.186 (1.9); 1.177 (4.5); | |
| | | 1.173 (4.1); 1.161 (1.3); 1.136 (5.4); 1.119 (11.4); | |
| | | 1.101 (5.2); 0.008 (1.5); 0.000 (32.6) | |
| 14 | 1.63[a] | Example 14: 1H-NMR(400.0 MHz, d6-DMSO): | N′-[4-(2,2-difluoro-1- |
| | | δ = 7.594 (0.4); 7.335 (1.3); 7.318 (9.8); 7.301 (4.8); | phenylcyclopropyl)-2,5- |
| | | 7.281 (1.6); 7.268 (0.4); 7.264 (0.3); 7.249 (1.0); | dimethylphenyl]-N-ethyl- |
| | | 7.235 (1.2); 7.230 (2.0); 7.224 (1.0); 7.220 (1.0); | N-methylmethanimidamide |
| | | 7.214 (1.8); 7.203 (4.2); 7.182 (0.7); 7.163 (0.6); | |
| | | 6.554 (2.9); 3.365 (0.4); 3.323 (48.2); 2.908 (2.9); | |
| | | 2.523 (0.8); 2.510 (19.3); 2.505 (39.8); 2.501 (52.9); | |
| | | 2.496 (38.0); 2.492 (18.3); 2.445 (0.5); 2.423 (0.7); | |
| | | 2.411 (0.5); 2.402 (0.5); 2.390 (0.4); 2.328 (0.3); | |
| | | 2.300 (2.5); 2.162 (16.0); 2.158 (15.9); 2.080 (0.4); | |
| | | 2.031 (0.7); 2.019 (0.5); 2.010 (0.5); 1.998 (0.9); | |
| | | 1.985 (0.6); 1.976 (0.5); 1.965 (0.4); 1.883 (0.5); | |
| | | 1.398 (7.1); 1.124 (4.7); 1.107 (10.0); 1.089 (4.5); | |
| | | 0.008 (1.8); 0.000 (48.7); −0.008 (2.1) | |
| 15 | 1.83[a] | Example 15: 1H-NMR(400.0 MHz, d6-DMSO): | N′-[4-(2,2-dichloro-1- |
| | | δ = 7.594 (0.9); 7.541 (0.6); 7.493 (7.2); 7.474 (8.7); | phenylcyclopropyl)-2,5- |
| | | 7.327 (4.5); 7.308 (9.0); 7.289 (5.3); 7.222 (3.4); | dimethylphenyl]-N-ethyl- |
| | | 7.204 (4.8); 7.185 (1.8); 6.520 (1.4); 5.755 (4.0); | N-methylmethanimidamide |
| | | 3.322 (58.4); 2.904 (7.0); 2.775 (0.5); 2.671 (0.9); | |
| | | 2.501 (125.5); 2.422 (0.6); 2.395 (0.6); 2.364 (0.6); | |
| | | 2.328 (1.4); 2.174 (8.5); 2.092 (1.3); 2.073 (1.2); | |
| | | 1.481 (0.4); 1.467 (0.4); 1.298 (0.3); 1.258 (0.6); | |
| | | 1.235 (1.1); 1.150 (0.4); 1.119 (7.8); 1.101 (16.0); | |
| | | 1.083 (7.7); 1.012 (0.4); 0.995 (0.4); 0.000 (61.9) | |
| 16 | 1.89[a] | Example 16: 1H-NMR(400.0 MHz, d6-DMSO): | N′-[4-(2,2-dibromo-1- |
| | | δ = 7.501 (7.0); 7.482 (7.7); 7.321 (3.2); 7.302 (6.4); | phenylcyclopropyl)-2,5- |
| | | 7.283 (3.8); 7.213 (3.0); 7.194 (4.2); 7.176 (1.7); | dimethylphenyl]-N-ethyl- |
| | | 6.460 (0.8); 5.754 (12.8); 3.319 (25.2); 2.894 (5.6); | N-methylmethanimidamide |
| | | 2.673 (0.4); 2.669 (0.5); 2.664 (0.4); 2.522 (2.0); | |
| | | 2.509 (30.6); 2.504 (62.0); 2.500 (82.3); 2.495 (59.5); | |
| | | 2.491 (29.2); 2.331 (0.6); 2.327 (0.7); 2.322 (0.6); | |
| | | 2.277 (0.5); 2.251 (1.0); 2.197 (3.7); 2.148 (3.3); | |
| | | 2.081 (0.7); 1.234 (0.6); 1.149 (0.3); 1.112 (7.5); | |
| | | 1.095 (16.0); 1.077 (7.2); 0.008 (0.7); 0.000 (20.4); | |
| | | −0.009 (0.9) | |
| 18 | 1.58^{[a]}; | Example 18: 1H-NMR(400.0 MHz, d6-DMSO): | N′-[5-chloro-2-methyl-4-(1- |
| | 5.32^{[b]} | δ = 7.6983 (0.9); 7.5942 (0.4); 7.2863 (1.9); 7.2674 | phenylethyl)phenyl]-N- |
| | | (5.1); 7.2493 (4.8); 7.2152 (6.3); 7.1973 (3.3); 7.1716 | ethyl-N- |
| | | (1.8); 7.1539 (2.6); 7.1391 (0.7); 7.1360 (1.0); 7.0963 | methylmethanimidamide |
| | | (5.7); 6.8146 (1.7); 5.7559 (1.1); 4.4578 (0.6); 4.4399 | |
| | | (2.1); 4.4219 (2.2); 4.4039 (0.6); 3.4069 (0.5); 3.3240 | |
| | | (8.2); 2.9586 (1.2); 2.9011 (3.2); 2.5051 (12.6); | |
| | | 2.5008 (16.0); 2.4964 (11.4); 2.1331 (16.0); 1.5393 | |
| | | (9.3); 1.5212 (9.1); 1.3964 (2.8); 1.1283 (3.5); 1.1107 | |
| | | (6.7); 1.0931 (3.3) | |
| 19 | 1.58^{[a]}; | Example 19: 1H-NMR(400.0 MHz, d6-DMSO): | N′-[5-chloro-2-methyl-4-(1- |
| | 5.24^{[b]} | δ = 7.7935 (1.3); 7.6874 (0.5); 7.3394 (1.1); 7.3348 | phenylethen-1-yl)phenyl]- |
| | | (1.7); 7.3309 (0.9); 7.3183 (4.9); 7.3137 (3.0); 7.2998 | N-ethyl-N- |
| | | (5.8); 7.2889 (1.5); 7.2848 (3.0); 7.2810 (2.4); 7.2744 | methylmethanimidamide |
| | | (1.1); 7.2678 (3.1); 7.2587 (0.9); 7.2497 (6.2); 7.2456 | |
| | | (6.6); 7.2329 (2.2); 7.2290 (4.2); 7.0860 (0.5); 7.0654 | |
| | | (0.7); 7.0482 (6.5); 6.8999 (1.7); 6.8479 (0.8); 6.8309 | |
| | | (0.6); 5.8120 (5.5); 5.8099 (5.5); 5.2060 (5.6); 5.2041 | |
| | | (5.5); 3.4443 (0.7); 3.4067 (0.4); 3.3682 (1.4); 3.3510 | |
| | | (1.5); 3.3201 (32.5); 3.0015 (1.7); 2.9390 (4.3); | |
| | | 2.6707 (0.4); 2.5233 (1.5); 2.5099 (25.7); 2.5057 | |
| | | (51.6); 2.5013 (67.7); 2.4968 (48.9); 2.3327 (0.4); | |
| | | 2.3281 (0.4); 2.1771 (16.0); 2.1377 (3.1); 1.3973 | |
| | | (0.5); 1.1573 (3.6); 1.1398 (7.5); 1.1224 (4.1); −0.0002 | |
| | | (6.4) | |
| 20 | 2.00^{[a]}; | Example 20: 1H-NMR(400.0 MHz, d6-DMSO): | N′-(4-benzyl-5-fluoro-2- |
| | 4.62^{[b]} | δ = 7.6963 (1.0); 7.5952 (0.4); 7.2864 (2.2); 7.2676 | methylphenyl)-N-ethyl-N- |
| | | (5.4); 7.2492 (5.3); 7.2320 (0.6); 7.1908 (6.7); 7.1848 | methylmethanimidamide |
| | | (3.8); 7.1716 (4.5); 7.1645 (3.7); 7.1461 (1.5); 6.9778 | |
| | | (3.1); 6.9556 (3.0); 6.6402 (1.0); 6.6110 (1.1); 6.3658 | |
| | | (0.4); 6.3352 (0.4); 4.9662 (0.6); 3.8334 (10.1); | |
| | | 3.7380 (1.0); 3.4108 (0.6); 3.3228 (10.2); 2.9568 | |
| | | (1.3); 2.9056 (3.3); 2.5053 (21.2); 2.5010 (26.7); | |
| | | 2.4968 (19.8); 2.0954 (16.0); 1.9625 (1.9); 1.1320 | |
| | | (3.6); 1.1144 (7.2); 1.0968 (3.6) | |
| 21 | 1.86^{[a]}; | Example 21: 1H-NMR(400.0 MHz, d6-DMSO): | N′-[2-chloro-4-[(2,4- |
| | 4.60^{[b]} | δ = 7.7077 (1.2); 7.5939 (0.5); 7.2451 (0.9); 7.2386 | difluorophenyl)methyl]-5- |
| | | (0.9); 7.2188 (1.4); 7.2151 (1.4); 7.1955 (1.0); 7.1892 | methylphenyl]-N-ethyl-N- |
| | | (1.0); 7.1512 (0.6); 7.1297 (1.5); 7.1123 (1.5); 7.0912 | methylmethanimidamide |
| | | (0.8); 7.0422 (1.0); 7.0369 (1.0); 7.0210 (1.6); 7.0157 | |
| | | (1.5); 6.9997 (0.7); 6.9943 (0.7); 6.9796 (5.3); 6.8071 | |
| | | (1.6); 3.8453 (7.2); 3.4173 (0.6); 3.3393 (1.3); 3.3200 | |
| | | (25.4); 2.9789 (1.5); 2.9128 (3.8); 2.6708 (0.4); | |
| | | 2.5061 (51.6); 2.5017 (63.8); 2.4973 (46.8); 2.3285 | |
| | | (0.4); 2.2292 (0.4); 2.1423 (16.0); 1.1446 (2.3); | |
| | | 1.1277 (4.4); 1.1106 (2.4); −0.0002 (0.5) | |
| 22 | 1.63^{[a]} | Example 22: 1H-NMR(400.0 MHz, d6-DMSO): | N′-[5-chloro-4-[(2,4- |
| | | δ = 7.7285 (1.1); 7.6207 (0.4); 7.2264 (1.1); 7.2199 | difluorophenyl)methyl]-2- |
| | | (1.2); 7.2015 (1.7); 7.1954 (2.2); 7.1770 (1.2); 7.1705 | methylphenyl]-N-ethyl-N- |
| | | (1.2); 7.1503 (0.8); 7.1289 (1.9); 7.1103 (2.2); 7.0901 | methylmethanimidamide |
| | | (1.1); 7.0257 (1.4); 7.0195 (1.4); 7.0082 (1.5); 7.0044 | |
| | | (1.6); 7.0017 (1.7); 6.9863 (1.1); 6.9796 (1.1); 6.9659 | |
| | | (5.8); 6.8807 (1.6); 5.7558 (3.7); 4.0390 (0.7); 4.0212 | |
| | | (0.7); 3.9230 (8.9); 3.4156 (0.6); 3.3420 (1.3); 3.3221 | |
| | | (7.3); 2.9767 (1.4); 2.9104 (3.6); 2.5115 (10.2); | |
| | | 2.5073 (19.8); 2.5028 (26.1); 2.4984 (19.6); 2.4943 | |
| | | (10.1); 2.1417 (0.8); 2.1078 (16.0); 1.9893 (2.8); | |
| | | 1.3974 (7.7); 1.1935 (0.8); 1.1757 (1.5); 1.1578 (0.9); | |
| | | 1.1382 (2.7); 1.1212 (5.1); 1.1041 (2.7); 0.0078 (0.7); | |
| | | −0.0002 (17.5); −0.0079 (0.8) | |
| 23 | 1.66^{[a]}; | Example 23: 1H-NMR(400.0 MHz, d6-DMSO): | N′-[5-chloro-2-methyl-4- |
| | 5.54^{[b]} | δ = 7.7422 (0.8); 7.1857 (1.0); 7.1802 (1.2); 7.1641 | [(2-methylphenyl)methyl] |
| | | (2.1); 7.1383 (0.6); 7.1339 (0.7); 7.1200 (2.0); 7.1150 | phenyl]-N-ethyl-N- |
| | | (2.2); 7.1047 (2.9); 7.0946 (1.9); 7.0895 (1.6); 7.0757 | methylmethanimidamide |
| | | (0.6); 7.0715 (0.5); 6.8889 (2.6); 6.8726 (1.9); 6.8672 | |
| | | (1.6); 6.7643 (4.6); 5.7551 (0.6); 3.8983 (8.5); 3.8046 | |
| | | (0.3); 3.4136 (0.5); 3.3504 (0.9); 3.3202 (9.7); 2.9807 | |
| | | (1.0); 2.9113 (2.5); 2.5055 (29.5); 2.5013 (38.1); | |
| | | 2.4970 (28.7); 2.2290 (16.0); 2.1408 (0.5); 2.0678 | |
| | | (12.7); 1.9883 (0.6); 1.9409 (0.6); 1.3976 (2.7); | |
| | | 1.1747 (0.3); 1.1563 (0.4); 1.1416 (1.9); 1.1246 (3.6); | |
| | | 1.1078 (2.1); 0.0075 (0.7); −0.0002 (17.6) | |
| 24 | 1.63^{[a]}; | Example 24: 1H-NMR(400.0 MHz, d6-DMSO): | N′-[5-chloro-4-[(2,3- |
| | 4.92^{[b]} | δ = 7.7338 (1.1); 7.6249 (0.4); 7.3091 (0.5); 7.3056 | difluorophenyl)methyl]-2- |
| | | (0.5); 7.2849 (1.3); 7.2633 (1.3); 7.2426 (0.7); 7.2392 | methylphenyl]-N-ethyl-N- |
| | | (0.6); 7.1459 (0.7); 7.1420 (0.7); 7.1326 (0.7); 7.1259 | methylmethanimidamide |
| | | (1.3); 7.1221 (1.3); 7.1125 (1.2); 7.1090 (1.4); 7.1018 | |
| | | (0.7); 7.0924 (0.6); 7.0889 (0.6); 7.0108 (5.5); 6.9130 | |
| | | (1.2); 6.8935 (3.0); 6.8777 (1.9); 4.0024 (9.0); 3.4151 | |
| | | (0.6); 3.3438 (1.3); 3.3220 (45.8); 2.9803 (1.3); | |
| | | 2.9114 (3.5); 2.6753 (0.3); 2.6708 (0.5); 2.6663 (0.3); | |
| | | 2.5241 (1.2); 2.5063 (64.3); 2.5020 (83.8); 2.4976 | |
| | | (60.1); 2.3330 (0.4); 2.3288 (0.5); 2.3244 (0.4); | |
| | | 2.1152 (16.0); 1.1395 (2.4); 1.1222 (4.7); 1.1051 | |
| | | (2.6); 0.0077 (1.8); −0.0002 (48.7); −0.0083 (1.9) | |
| 25 | 2.05^{[a]}; | Example 25: 1H-NMR(400.0 MHz, d6-DMSO): δ = | N′-[5-chloro-2-methyl-4- |
| | 6.02^{[b]} | 7.7120 (0.9); 7.6035 (0.4); 7.4517 (1.8); 7.4345 (5.2); | [(2-phenylphenyl)methyl] |
| | | 7.4157 (4.7); 7.3876 (2.4); 7.3752 (0.8); 7.3697 (2.8); | phenyl]-N-ethyl-N- |
| | | 7.3627 (0.6); 7.3513 (0.8); 7.3200 (4.7); 7.3162 (5.7); | methylmethanimidamide |
| | | 7.2991 (4.7); 7.2929 (4.3); 7.2840 (3.6); 7.2788 (3.9); | |
| | | 7.2701 (5.3); 7.2603 (0.9); 7.2389 (0.6); 7.2289 (2.9); | |
| | | 7.2200 (2.1); 7.2153 (1.6); 7.2062 (1.5); 7.0425 (0.4); | |
| | | 7.0339 (2.0); 7.0231 (1.7); 7.0200 (1.8); 7.0113 (1.8); | |
| | | 6.8164 (1.5); 6.7179 (5.8); 3.8752 (10.6); 3.4058 | |
| | | (0.6); 3.3222 (43.7); 2.9683 (1.2); 2.9001 (3.1); | |
| | | 2.6708 (0.5); 2.6664 (0.4); 2.5238 (1.4); 2.5061 | |
| | | (71.3); 2.5018 (91.3); 2.4975 (65.1); 2.3330 (0.4); | |
| | | 2.3286 (0.5); 2.0571 (16.0); 1.9885 (0.8); 1.3976 | |
| | | (11.6); 1.1748 (0.4); 1.1309 (2.5); 1.1140 (4.9); | |
| | | 1.0967 (2.6); 0.0079 (1.9); −0.0002 (51.6); −0.0084 | |
| | | (2.0) | |
| 26 | 1.53^{[a]}; | Example 26: 1H-NMR(400.0 MHz, d6-DMSO): | N′-[2-chloro-4-[(2- |
| | 4.45^{[b]} | δ = 7.7010 (0.6); 7.2282 (0.5); 7.2199 (0.5); 7.2137 | methoxyphenyl)methyl]-5- |
| | | (0.6); 7.2065 (0.9); 7.2000 (0.6); 7.1927 (0.8); 7.1848 | methylphenyl]-N-ethyl-N- |
| | | (0.7); 7.0002 (2.0); 6.9797 (1.6); 6.9035 (4.1); 6.8754 | methylmethanimidamide |
| | | (0.4); 6.8648 (4.0); 6.8571 (1.9); 6.8503 (1.6); 6.7849 | |
| | | (1.0); 5.7567 (0.4); 3.7833 (16.0); 3.7761 (6.4); | |
| | | 3.3364 (0.7); 3.3230 (8.6); 2.9744 (0.8); 2.9100 (2.0); | |
| | | 2.5097 (6.0); 2.5055 (12.3); 2.5010 (16.4); 2.4966 | |
| | | (11.8); 2.4924 (5.7); 2.1333 (11.0); 1.3971 (0.6); | |
| | | 1.1431 (1.5); 1.1255 (3.1); 1.1079 (1.6); −0.0002 (8.5) | |
| 27 | 1.61^{[a]}; | Example 27: 1H-NMR(400.0 MHz, d6-DMSO): | N′-[2-chloro-5-methyl-4-(1- |
| | 4.70^{[b]} | δ = 7.6897 (0.9); 7.5799 (0.3); 7.2897 (1.7); 7.2689 | phenylethyl)phenyl]-N- |
| | | (3.7); 7.2579 (0.9); 7.2518 (3.5); 7.1758 (7.2); 7.1574 | ethyl-N- |
| | | (5.7); 7.1505 (6.5); 7.1418 (0.8); 7.1387 (0.9); 6.7447 | methylmethanimidamide |
| | | (1.4); 4.2427 (0.4); 4.2248 (1.6); 4.2069 (1.6); 4.1889 | |
| | | (0.5); 3.4202 (0.4); 3.4131 (0.4); 3.3221 (17.9); | |
| | | 2.9676 (1.1); 2.9082 (3.0); 2.5233 (0.4); 2.5099 | |
| | | (12.2); 2.5055 (25.4); 2.5011 (33.7); 2.4966 (23.9); | |
| | | 2.4923 (11.3); 2.1163 (16.0); 1.5051 (7.0); 1.4872 | |
| | | (7.0); 1.3973 (1.1); 1.1380 (2.8); 1.1203 (5.8); 1.1025 | |
| | | (2.8); 0.0079 (0.6); −0.0002 (18.3); −0.0086 (0.6) | |
| 28 | 1.63^{[a]}; | Example 28: 1H-NMR(400.0 MHz, d6-DMSO): δ = | N′-[2-chloro-4-[(2- |
| | 4.93^{[b]} | 7.7254 (1.1); 7.6110 (0.4); 7.4875 (1.7); 7.4769 (1.3); | chlorophenyl)methyl]-5- |
| | | 7.4729 (1.7); 7.4643 (2.2); 7.4542 (0.4); 7.2998 (0.5); | methylphenyl]-N-ethyl-N- |
| | | 7.2894 (4.2); 7.2806 (2.9); 7.2753 (3.2); 7.2661 (4.0); | methylmethanimidamide |
| | | 7.2565 (0.5); 7.0545 (1.6); 7.0455 (1.5); 7.0415 (1.3); | |
| | | 7.0311 (1.3); 6.8502 (6.0); 6.8354 (1.5); 3.9433 (8.7); | |
| | | 3.4196 (0.5); 3.3464 (1.0); 3.3243 (13.9); 2.9851 | |
| | | (1.3); 2.9165 (3.4); 2.5240 (0.4); 2.5064 (18.9); | |
| | | 2.5020 (24.4); 2.4976 (17.3); 2.1350 (16.0); 1.3971 | |
| | | (0.6); 1.1486 (2.0); 1.1312 (3.9); 1.1139 (2.1); 0.0078 | |
| | | (0.4); −0.0002 (10.8); −0.0086 (0.4) | |
| 29 | 1.58^{[a]}; | Example 29: 1H-NMR(400.0 MHz, d6-DMSO): δ = | N′-[5-chloro-4-+(2- |
| | 4.95^{[b]} | 7.7263 (0.5); 7.2147 (0.5); 7.2056 (0.5); 7.2011 (0.7); | methoxyphenyl)methyl]-2- |
| | | 7.1933 (0.9); 7.1857 (0.6); 7.1801 (0.8); 7.1715 (0.7); | methylphenyl]-N-ethyl-N- |
| | | 6.9889 (2.1); 6.9685 (1.6); 6.8819 (3.5); 6.8618 (0.9); | methylmethanimidamide |
| | | 6.8546 (1.0); 6.8441 (4.7); 6.8354 (2.2); 6.8307 (2.0); | |
| | | 3.8692 (6.6); 3.7938 (16.0); 3.3438 (0.6); 3.3213 | |
| | | (14.8); 2.9739 (0.6); 2.9088 (1.6); 2.5236 (0.5); | |
| | | 2.5099 (14.2); 2.5057 (29.3); 2.5012 (39.0); 2.4968 | |
| | | (28.0); 2.4927 (13.6); 2.0829 (9.6); 1.1387 (1.4); | |
| | | 1.1213 (2.8); 1.1039 (1.5); −0.0002 (5.7) | |
| 30 | 1.81^{[a]}; | Example 30: 1H-NMR(400.0 MHz, d6-DMSO): δ = | N′-[5-chloro-4-[(2- |
| | 5.55^{[b]} | 7.7501 (1.1); 7.6411 (0.6); 7.4724 (2.1); 7.4632 (2.1); | chlorophenyl)methyl]-2- |
| | | 7.4579 (2.2); 7.4493 (2.6); 7.2827 (0.9); 7.2727 (5.1); | methylphenyl]-N-ethyl-N- |
| | | 7.2640 (4.4); 7.2581 (4.5); 7.2494 (5.0); 7.2402 (0.7); | methylmethanimidamide |
| | | 7.0383 (0.5); 7.0288 (2.1); 7.0197 (1.9); 7.0144 (1.8); | |
| | | 7.0052 (1.7); 6.9097 (1.6); 6.8677 (5.8); 4.0295 | |
| | | (11.5); 3.4165 (0.8); 3.3208 (229.2); 2.9798 (1.4); | |
| | | 2.9665 (0.9); 2.9146 (3.5); 2.6703 (3.1); 2.6100 (0.4); | |
| | | 2.5055 (412.8); 2.5012 (531.9); 2.4969 (384.4); | |
| | | 2.3279 (3.0); 2.0912 (16.0); 1.2330 (0.5); 1.1422 | |
| | | (2.4); 1.1267 (4.5); 1.1100 (2.7); 1.0875 (0.4); 0.1465 | |
| | | (0.5); 0.0078 (4.4); −0.0002 (118.1); −0.0083 (5.1); | |
| | | −0.1492 (0.5) | |
| 31 | 1.66^{[a]}; | Example 31: 1H-NMR(400.0 MHz, d6-DMSO): | N′-[2-chloro-5-methyl-4-(1- |
| | 4.93^{[b]} | δ = 7.7789 (1.5); 7.6677 (0.7); 7.3558 (1.2); 7.3390 | phenylethen-1-yl)phenyl]- |
| | | (3.7); 7.3204 (4.3); 7.3076 (2.4); 7.2908 (1.9); 7.2719 | N-ethyl-N- |
| | | (0.6); 7.2484 (4.8); 7.2315 (3.5); 7.0846 (6.0); 6.8436 | methylmethanimidamide |
| | | (1.9); 5.7855 (4.3); 5.1762 (4.3); 3.4620 (0.6); 3.4452 | |
| | | (0.8); 3.3665 (1.7); 3.3265 (170.4); 3.0056 (2.0); | |
| | | 2.9439 (4.9); 2.6711 (1.0); 2.5055 (136.5); 2.5016 | |
| | | (169.0); 2.4979 (131.0); 2.3281 (1.0); 1.8954 (16.0); | |
| | | 1.1638 (2.9); 1.1467 (5.9); 1.1290 (2.9); −0.0001 (2.2) | |
| 32 | 1.46^{[a]}; | Example 32: 1H-NMR(400.0 MHz, d6-DMSO): | N-ethyl-N′-[5-fluoro-4-[(2- |
| | 4.34^{[b]} | δ = 7.7009 (0.4); 7.2062 (0.5); 7.2017 (0.6); 7.1829 | methoxyphenyl)methyl]-2- |
| | | (1.0); 7.1672 (0.7); 7.1629 (0.8); 6.9728 (1.6); 6.9528 | methylphenyl]-N- |
| | | (2.3); 6.9335 (1.3); 6.8575 (2.2); 6.8415 (1.7); 6.8392 | methylmethanimidamide |
| | | (1.8); 6.8347 (1.5); 6.8231 (0.7); 6.8211 (0.7); 6.6329 | |
| | | (0.5); 6.6026 (0.5); 3.7854 (16.0); 3.7787 (5.8); | |
| | | 3.3232 (66.9); 2.9542 (0.5); 2.9073 (1.3); 2.5237 | |
| | | (0.7); 2.5187 (1.2); 2.5103 (17.9); 2.5059 (37.1); | |
| | | 2.5014 (49.4); 2.4969 (35.4); 2.4925 (17.2); 2.0728 | |
| | | (7.6); 1.9884 (0.8); 1.9433 (0.4); 1.3978 (4.2); 1.1746 | |
| | | (0.4); 1.1349 (1.5); 1.1173 (3.1); 1.0998 (1.6); −0.0002 | |
| | | (6.1) | |
| 33 | 1.41^{[a]}; | Example 33: 1H-NMR(400.0 MHz, d6-DMSO): | N-ethyl-N′-[5-fluoro-4-[(2- |
| | 4.37^{[b]} | δ = 7.7033 (1.0); 7.6009 (0.4); 7.2818 (0.5); 7.2773 | fluorophenyl)methyl]-2- |
| | | (0.5); 7.2680 (0.6); 7.2629 (1.3); 7.2435 (1.8); 7.2389 | methylphenyl]-N- |
| | | (1.2); 7.2300 (0.9); 7.2251 (1.0); 7.2041 (0.9); 7.1867 | methylmethanimidamide |
| | | (2.1); 7.1676 (3.3); 7.1419 (2.3); 7.1336 (2.7); 7.1210 | |
| | | (1.6); 7.1153 (3.1); 7.0968 (1.2); 7.0943 (1.1); 6.9233 | |
| | | (2.8); 6.9012 (2.8); 6.6512 (1.0); 6.6229 (1.1); 4.0379 | |
| | | (0.4); 4.0203 (0.4); 3.8584 (9.1); 3.7610 (0.4); 3.4093 | |
| | | (0.5); 3.3210 (28.0); 2.9605 (1.2); 2.9058 (3.3); | |
| | | 2.6706 (0.3); 2.5102 (20.8); 2.5060 (41.4); 2.5015 | |
| | | (54.4); 2.4970 (39.4); 2.4928 (19.5); 2.0861 (16.0); | |
| | | 1.9886 (1.5); 1.9541 (0.7); 1.3974 (10.0); 1.1926 | |
| | | (0.4); 1.1748 (0.8); 1.1570 (0.5); 1.1330 (3.2); 1.1155 | |
| | | (6.2); 1.0980 (3.2); −0.0002 (6.9) | |
| 34 | 1.55^{[a]}; | Example 34: 1H-NMR(400.0 MHz, d6-DMSO): | N-ethyl-N′-[5-fluoro-2- |
| | 4.63^{[b]} | δ = 7.6807 (0.9); 7.5811 (0.3); 7.2860 (1.8); 7.2668 | methyl-4-(1- |
| | | (5.4); 7.2490 (6.0); 7.2248 (6.9); 7.2076 (3.0); 7.1740 | phenylethyl)phenyl]-N- |
| | | (1.4); 7.1703 (1.9); 7.1666 (1.1); 7.1527 (3.0); 7.1388 | methylmethanimidamide |
| | | (0.8); 7.1351 (1.3); 7.0449 (2.9); 7.0228 (2.9); 6.5861 | |
| | | (1.1); 6.5557 (1.1); 4.9435 (0.5); 4.2982 (0.6); 4.2804 | |
| | | (2.2); 4.2623 (2.2); 4.2440 (0.7); 3.4019 (0.5); 3.3192 | |
| | | (67.6); 2.9907 (0.3); 2.9211 (2.0); 2.9015 (3.0); | |
| | | 2.6744 (0.6); 2.6698 (0.8); 2.6654 (0.6); 2.5232 (2.0); | |
| | | 2.5097 (49.0); 2.5053 (101.8); 2.5009 (135.8); 2.4963 | |
| | | (98.5); 2.4920 (48.6); 2.3320 (0.6); 2.3275 (0.8); | |
| | | 2.3231 (0.6); 2.1170 (16.0); 1.9878 (1.3); 1.9838 | |
| | | (1.5); 1.5459 (9.9); 1.5277 (9.9); 1.4982 (0.9); 1.4799 | |
| | | (0.8); 1.3975 (8.1); 1.1743 (0.5); 1.1563 (0.4); 1.1275 | |
| | | (4.3); 1.1098 (8.2); 1.0921 (3.9); 0.0077 (0.5); −0.0004 | |
| | | (16.0); −0.0084 (0.7) | |
| 35 | 1.52^{[a]}; | Example 35: 1H-NMR(400.0 MHz, d6-DMSO): | N′-[4-[(2,4- |
| | 4.54^{[b]} | δ = 7.7043 (1.0); 7.6008 (0.4); 7.3496 (0.4); 7.3287 | difluorophenyl)methyl]-5- |
| | | (0.4); 7.2552 (0.8); 7.2338 (1.8); 7.2165 (1.9); 7.2103 | fluoro-2-methylphenyl]-N- |
| | | (2.1); 7.2032 (1.4); 7.1949 (1.1); 7.1842 (1.8); 7.1790 | ethyl-N- |
| | | (1.8); 7.1602 (1.2); 7.1539 (1.3); 7.0339 (1.0); 7.0286 | methylmethanimidamide |
| | | (1.0); 7.0127 (1.9); 7.0074 (1.8); 6.9915 (0.9); 6.9862 | |
| | | (0.8); 6.9192 (2.8); 6.8969 (2.8); 6.6535 (1.0); 6.6234 | |
| | | (1.1); 3.8318 (8.6); 3.4124 (0.6); 3.3184 (55.2); | |
| | | 2.9622 (1.3); 2.9206 (2.2); 2.9055 (3.4); 2.6749 (0.7); | |
| | | 2.6703 (1.0); 2.6659 (0.7); 2.5234 (3.1); 2.5100 | |
| | | (61.0); 2.5058 (123.5); 2.5013 (163.4); 2.4968 | |
| | | (119.1); 2.4926 (59.3); 2.3327 (0.7); 2.3280 (1.0); | |
| | | 2.3235 (0.7); 2.1332 (1.6); 2.0877 (16.0); 1.9884 | |
| | | (0.8); 1.9548 (0.4); 1.3977 (3.2); 1.1747 (0.4); 1.1569 | |
| | | (0.4); 1.1334 (3.5); 1.1158 (6.3); 1.0984 (3.2); 0.1459 | |
| | | (0.9); 0.0078 (7.9); −0.0002 (199.2); −0.0084 (9.3); | |
| | | −0.1497 (0.9) | |

^{[a]}LogP value is determined by measurement of LC-UV, in an acidic range, with 0.1% formic acid in water and acetonitrile as eluent (linear gradient from 10% acetonitrile to 95% acetonitrile).
^{[b]}LogP value is determined by measurement of LC-UV, in a neutral range, with 0.001 molar ammonium acetate solution in water and acetonitrile as eluent (linear gradient from 10% acetonitrile to 95% acetonitrile).
^{[c]}LogP value is determined by measurement of LC-UV, in an acidic range, with 0.1% phosphoric acid and acetonitrile as eluent (linear gradient from 10% acetonitrile to 95% acetonitrile).

### Log P Measurement

Measurement of Log P values was performed according to EEC directive 79/831 Annex V.A8 by HPLC (High Performance Liquid Chromatography) on reversed phase columns with the following methods:

If more than one Log P value is available within the same method, all the values are given and separated by “+”.

Calibration was done with straight-chain alkan2-ones (with 3 to 16 carbon atoms) with known Log P values (measurement of Log P values using retention times with linear interpolation between successive alkanones). Lambda-max-values were determined using UV-spectra from 200 nm to 400 nm and the peak values of the chromatographic signals.

### NMR-Peak Lists

1H-NMR data of selected examples are written in form of 1H-NMR-peak lists. To each signal peak are listed the δ-value in ppm and the signal intensity in round brackets. Between the δ-value—signal intensity pairs are semicolons as delimiters.

The peak list of an example has therefore the form:

δ₁ (intensity₁; δ₂ (intensity₂); . . . ; δᵢ (intensityᵢ); . . . ; δₙ (intensityₙ)

Intensity of sharp signals correlates with the height of the signals in a printed example of a NMR spectrum in cm and shows the real relations of signal intensities. From broad signals several peaks or the middle of the signal and their relative intensity in comparison to the most intensive signal in the spectrum can be shown.

For calibrating chemical shift for 1H spectra, we use tetramethylsilane and/or the chemical shift of the solvent used, especially in the case of spectra measured in DMSO. Therefore in NMR peak lists, tetramethylsilane peak can occur but not necessarily.

The 1H-NMR peak lists are similar to classical 1H-NMR prints and contains therefore usually all peaks, which are listed at classical NMR-interpretation.

Additionally they can show like classical 1H-NMR prints signals of solvents, stereoisomers of the target compounds, which are also object of the invention, and/or peaks of impurities.

To show compound signals in the delta-range of solvents and/or water the usual peaks of solvents, for example peaks of DMSO in DMSO-D₆ and the peak of water are shown in our 1H-NMR peak lists and have usually on average a high intensity.

The peaks of stereoisomers of the target compounds and/or peaks of impurities have usually on average a lower intensity than the peaks of target compounds (for example with a purity>90%).

Such stereoisomers and/or impurities can be typical for the specific preparation process. Therefore their peaks can help to recognize the reproduction of our preparation process via “side-products-fingerprints”.

The present invention will be illustrated with the biological examples. However the invention is not limited to the examples.

### Example: In Vivo Preventive Test on Phakopsora Test (Soybeans)

| | |
|---|---|
| Solvent: | 24.5 parts by weight of acetone |
| | 24.5 parts by weight of dimethylacetamide |
| Emulsifier: | 1 part by weight of alkylaryl polyglycol ether |

To produce a suitable preparation of active compound, 1 part by weight of active compound is mixed with the stated amounts of solvent and emulsifier, and the concentrate is diluted with water to the desired concentration.

To test for preventive activity, young plants are sprayed with the preparation of active compound at the stated rate of application. After the spray coating has dried on, the plants are inoculated with an aqueous spore suspension of the causal agent of soybean rust (Phakopsora pachyrhizi) and stay for 24 h without light in an incubation cabinet at approximately 24° C. and a relative atmospheric humidity of 95%.

The plants remain in the incubation cabinet at approximately 24° C. and a relative atmospheric humidity of approximately 80% and a day/night interval of 12 h.

The test is evaluated 7 days after the inoculation. 0% means an efficacy which corresponds to that of the untreated control, while an efficacy of 100% means that no disease is observed.

In this test the following compounds according to the invention showed efficacy between 80% and 89% at a concentration of 10 ppm of active ingredient: 1; 19

In this test the following compounds according to the invention showed efficacy between 90% and 100% at a concentration of 10 ppm of active ingredient: 2; 3; 6; 8; 9; 10; 11; 12; 13; 14; 18; 20; 21; 22; 23; 24; 30

1. A compound of formula (I) 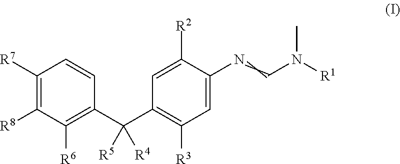
wherein:
R¹ is selected from the group consisting of C₁-C₈-alkyl and C₃-C₇-cycloalkyl which may be independently non-substituted or substituted by one or more group(s) selected from the group consisting of halogen and C₁-C₈-alkoxy;
R² and R³ are each independently selected from the group consisting of halogen, cyano, C₁-C₈-alkyl, C₃-C₇-cycloalkyl, —O—C₁-C₈-alkyl, —C₂-C₈-alkenyl, —C₂-C₈-alkynyl, —Si(R^{3a})(R^{3b})(R^{3c}), —C(O)—C₁-C₈-alkyl, —C(O)—C₃-C₇-cycloalkyl, —C(O)NH—C₁-C₈-alkyl, —C(O)N-di-C₁-C₈-alkyl, —C(O)O—C₁-C₈-alkyl, —S(O)ₙ—C₁-C₈-alkyl, —NH—C₁-C₈-alkyl, and —N-di-C₁-C₈-alkyl, which may be independently non-substituted or substituted by one or more group(s) selected from the group consisting of halogen and C₁-C₈-alkoxy;
wherein R^{3a}, R^{3b}, and R^{3c} are independently from each other phenyl or C₁-C₈-alkyl; and
n is 0, 1 or 2;
R⁴, R⁵, R⁶, R⁷ and R⁸ are each independently selected from the group consisting of H, halogen, cyano, C₁-C₈-alkyl, C₃-C₇-cycloalkyl, —O—C₁-C₈-alkyl, —C₂-C₈-alkenyl, —C₂-C₈-alkynyl, —Si(R^{3a})(R^{3b})(R^{3c}), —C(O)—C₁-C₈-alkyl, —C(O)—C₃-C₇-cycloalkyl, —C(O)NH—C₁-C₈-alkyl, —C(O)N-di-C₁-C₈-alkyl, —C(O)O—C₁-C₈-alkyl, —S(O)ₙ—C₁-C₈-alkyl, —NH—C₁-C₈-alkyl, —N-di-C₁-C₈-alkyl, and C₆-C₁₄-aryl, which may be independently non-substituted or substituted by one or more group(s) selected from the group consisting of halogen, methyl, halomethyl, and C₁-C₈-alkoxy;
wherein R^{3a}, R^{3b}, and R^{3c} are independently from each other phenyl or C₁-C₈-alkyl; and
n is 0, 1 or 2;
or in which R⁴ and R⁵ can form, together with the atom to which they are bonded or with additional atoms chosen from N, O, P and S, a 3- to 7-membered ring selected from the group consisting of cycloalkyl and heterocyclyl, which may optionally be substituted by one or more halogen group(s), and wherein R⁶, R⁷ and R⁸ are as defined above;
or in which R⁴ and R⁵ together can form a double bonded substituent ═CR⁹R¹⁰, wherein R⁹ and R¹⁰ are each independently selected from the group consisting of H, halogen, Me and Et, and wherein R⁶, R⁷ and R⁸ are as defined above;
or a salt, an N-oxide, or a metal complex thereof, or a stereoisomer of any of the foregoing.

2. The compound according to claim 1, wherein
R¹ is C₁-C₈-alkyl,
R² is selected from the group consisting of halogen, cyano, and C₁-C₈-alkyl which may be independently non-substituted or substituted by one or more group(s) selected from the group consisting of halogen and C₁-C₈-alkoxy;
R³ is selected from the group consisting of halogen, cyano, and C₁-C₈-alkyl which may be independently non-substituted or substituted by one or more group(s) selected from the group consisting of halogen and C₁-C₈-alkoxy;
R⁴ and R⁵ are selected from the group consisting of H, halogen, cyano, and C₁-C₈-alkyl which may be independently non-substituted or substituted by one or more group(s) selected from the group consisting of halogen and C₁-C₈-alkoxy;
or R⁴ and R⁵ can form, together with the atom to which they are bonded or with additional atoms chosen from N, O, P and S, a 3- to 7-membered ring selected from the group consisting of cycloalkyl and heterocyclyl, which may optionally be substituted by one or more halogen group(s);
or in which R⁴ and R⁵ together can form a double bonded substituent ═CR⁹R¹⁰, wherein R⁹ and R¹⁰ are each independently selected from the group consisting of H, F, Cl, Me and Et;
R⁶, R⁷ and R⁸ are independently selected from the group consisting of H, F, Cl, cyano, Me, methoxy, phenyl and phenyl substituted by one or more substituents selected from the group consisting of halogen, Me and CF₃;
or a salt, an N-oxide, or a metal complex thereof, or a stereoisomer of any of the foregoing.

3. The compound according to claim 1, wherein
R¹ is selected from the group consisting of Me, Et, and iPr;
R² is selected from the group consisting of Me, cyano, Cl, Br, I, CHF₂, and CF₃;
R³ is selected from the group consisting of Me, Cyano, F, Cl, Br, and I;
R⁴ and R⁵ are each H;
or R⁴ and R⁵ can form, together with the atom to which they are bonded or with additional atoms chosen from N, O, P and S, a 3- to 7-membered ring selected from the group consisting of cycloalkyl and heterocyclyl, which may optionally be substituted by one or more halogen group(s);
or in which R⁴ and R⁵ together can form a double bonded substituent ═CH₂;
R⁶ is selected from the group consisting of H, Me, cyano, and F;
R⁷ and R⁸ are each H;
or a salt, an N-oxide, or a metal complex thereof, or a stereoisomer of any of the foregoing.

4. The compound according to claim 1, wherein
R^{l} is C₁-C₈-alkyl,
R² is selected from the group consisting of halogen, cyano, and C₁-C₈-alkyl which may be independently non-substituted or substituted by one or more halogen group(s);
R³ is selected from the group consisting of halogen, cyano, and C₁-C₈-alkyl which may be independently non-substituted or substituted by one or more halogen group(s);
R⁴ and R⁵ are selected from the group consisting of H, halogen, cyano, and C₁-C₈-alkyl which may be independently non-substituted or substituted by one or more halogen group(s);
or R⁴ and R⁵ can form, together with the atom to which they are bonded a 3- to 6-membered cycloalkyl ring, which may optionally be substituted by one or more halogen group(s);
or in which R⁴ and R⁵ together can form a double bonded substituent ═CR⁹R¹⁰, wherein R⁹ and R¹⁰ are each independently selected from the group consisting of hydrogen, Me and Et;
R⁶, R⁷ and R⁸ are independently selected from the group consisting of H, F, Cl, cyano, Me, methoxy and phenyl;
or a salt, an N-oxide, or a metal complex thereof, or a stereoisomer of any of the foregoing.

5. The compound according to claim 1, wherein
R¹ is selected from the group consisting of Me, Et, and iPr;
R² is selected from the group consisting of Me, cyano, Cl, Br, I, CHF₂, and CF₃;
R³ is selected from the group consisting of Me, iPr, Cyano, F, Cl, Br, and I;
R⁴ and R⁵ are each independently selected from the group consisting of H and Me;
or R⁴ and R⁵ can form, together with the atom to which they are bonded a cyclopropyl, which may optionally be substituted by one or more group(s) selected from the group consisting of F, Cl and Br;
or in which R⁴ and R⁵ together can form a double bonded substituent ═CH₂;
R⁶ is selected from the group consisting of H, Me, cyano, F, Cl, methoxy and phenyl;
R⁷ is selected from the group consisting of H and F, and
R⁸ is selected from the group consisting of H and F;
or a salt, an N-oxide, or a metal complex thereof, or a stereoisomer of any of the foregoing.

6. The compound according to claim 1, wherein
R¹ is selected from the group consisting of Et and iPr;
R² is selected from the group consisting of Me and Cl;
R³ is selected from the group consisting of Me, F and Cl;
R⁴ is selected from the group consisting of H and Me, and
R⁵ is H;
or R⁴ and R⁵ can form, together with the atom to which they are bonded a cyclopropyl, which may optionally be substituted by one or two F;
or in which R⁴ and R⁵ together can form a double bonded substituent ═CH₂;
R⁶ is selected from the group consisting of H, Me, cyano, F and Cl;
R⁷ is selected from the group consisting of H and F, and
R⁸ is selected from the group consisting of H and F;
or a salt, an N-oxide, or a metal complex thereof, or a stereoisomer of any of the foregoing.

7. A process for preparing a compound as claimed in claim 1 which comprises at least one of the following steps (a) to (g):
(a) reacting an aniline derivative of formula (II) to afford a derivative of formula (III) according to the reaction scheme below: 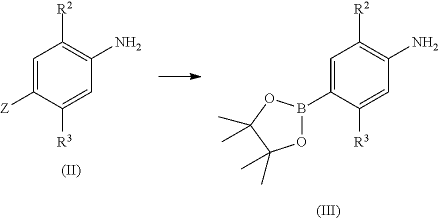
(b) reacting a derivative of formula (III) with a benzyl derivative of formula (IV) to afford a derivative of formula (V) in accordance with the reaction scheme below: 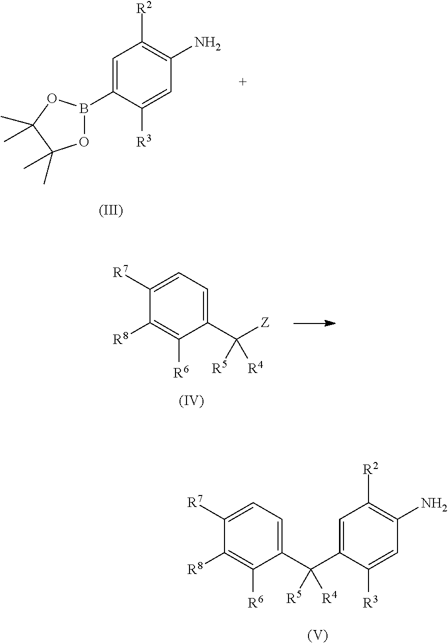
(c) coupling a nitrobenzene derivative of formula (VI) with a boronic acid or an ester of formula (VII) to afford an alkenyl derivative of formula (VIII) according to the reaction scheme below: 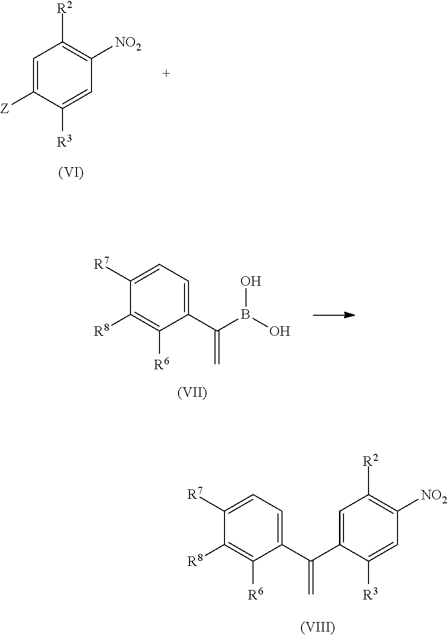
(d) reacting an alkenyl derivative of formula (VIII) to afford a cyclopropyl derivative of formula (IX) according to the reaction scheme below: 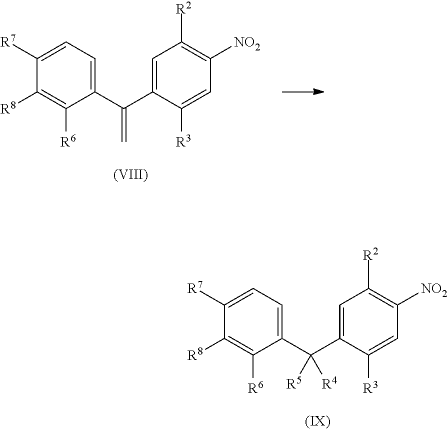
(e) reducing a nitrobenzene derivative of formula (IX) to an aniline derivative of formula (V) according to the reaction scheme below: 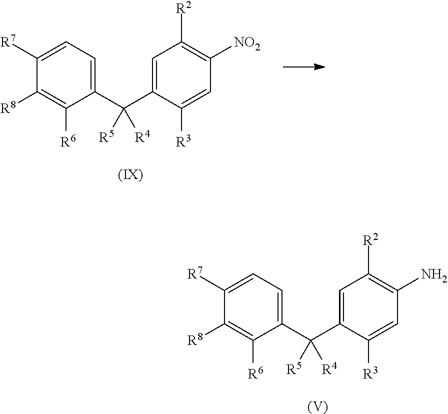
(f) reacting an aniline of formula (V) with an aminoacetal to afford an amidine of formula (I) according to the scheme below: 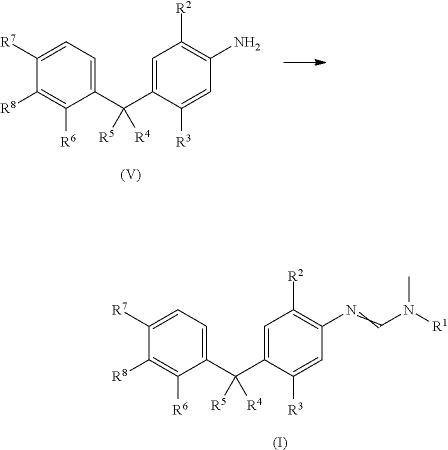
(g) reacting an organometallic compound of formula (X) with an aniline derivative of formula (II) to afford an aniline of formula (V) according to the scheme below: 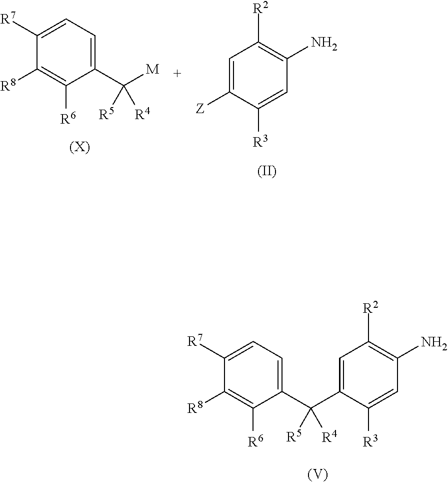
where in the above schemes:
Z is selected from the group consisting of Cl, Br, I and OSO₂CF₃;
M is selected from the group consisting of MgZ and ZnZ;
R¹ to R⁸ have the meanings as in claim 1.

8. A composition comprising the compound as claimed in claim 1, or a salt, an N-oxide, or a metal complex thereof, or a stereoisomer of any of the foregoing, and further comprising an auxiliary, a solvent, a carrier, a surfactant, or an extender.

9. (canceled)

10. A method for controlling phytopathogenic fungi in crop protection, comprising applying the compound as claimed in claim 1, or a salt, an N-oxide, or a metal complex thereof, or a stereoisomer of any of the foregoing, to the phytopathogenic fungi and/or their habitat.

11. A seed comprising the compound as claimed in claim 1, or a salt, an N-oxide, or a metal complex thereof, or a stereoisomer of any of the foregoing.

12. A method for treating a seed comprising applying the compound as claimed in claim 1, or a salt, an N-oxide, or a metal complex thereof, or a stereoisomer of any of the foregoing, to the seed.

13. A method for treating a transgenic plant comprising applying the compound as claimed in claim 1, or a salt, an N-oxide, or a metal complex thereof, or a stereoisomer of any of the foregoing, to the transgenic plant.

14. A method for treating a seed of a transgenic plant comprising applying the compound as claimed in claim 1, or a salt, an N-oxide, or a metal complex thereof, or a stereoisomer of any of the foregoing, to the seed of the transgenic plant.

15. A method for protecting a seed against phytopathogenic fungi comprising treating the seed with at least one compound as claimed in claim 1, or a salt, an N-oxide, or a metal complex thereof, or a stereoisomer of any of the foregoing.

16. A method for controlling phytopathogenic fungi in crop protection, comprising applying the composition according to claim 8 to the phytopathogenic fungi and/or their habitat.

17. A seed comprising the composition according to claim 8.

18. A method for treating a seed comprising applying the composition according to claim 8 to the seed.

19. A method for treating a transgenic plant comprising applying the composition according to claim 8 to the transgenic plant.

20. A method for treating a seed of a transgenic plant comprising applying the composition according to claim 8 to the seed of the transgenic plant.

21. A method for protecting seed against phytopathogenic comprising treating the seed with the composition according to claim 8.
